(19) United States
(12) Patent Application Publication
(10) Pub. No.: US 2024/0139306 A1
(43) Pub. Date: May 2, 2024

(54) METHODS AND COMPOSITIONS FOR DETECTING AND PRODUCING PORCINE MORBILLIVIRUS AND VACCINES THEREOF

(71) Applicant: Iowa State University Research Foundation, Inc., Ames, IA (US)

(72) Inventors: Ganwu Li, Ames, IA (US); Bailey Arruda, Ames, IA (US); Huigang Shen, Ames, IA (US); Ying Zheng, Ames, IA (US)

(21) Appl. No.: 18/279,314

(22) PCT Filed: Mar. 1, 2022
(86) PCT No.: PCT/US2022/018319

§ 371 Date: Aug. 29, 2023

Related U.S. Application Data
(60) Provisional application No. 63/200,313, filed on Mar. 1, 2021.

(51) Int. Cl. A61K 39/155; C07K 16/10; C12N 7/00; G01N 33/569; G01N 33/58; A61K 39/00

(52) U.S. Cl. CPC A61K 39/155; C07K 16/1027; C12N 7/00; G01N 33/56983; G01N 33/581; A61K 2039/552; C12N 2760/18422; C12N 2760/18434; C12N 2760/18451; G01N 2333/115

(57) ABSTRACT

Disclosed herein are methods for detecting and producing PoMV. Further, disclosed herein are immunogenic and/or vaccine compositions and methods for treating or preventing PoMV. The compositions and methods include immunogenic portions of PoMV including entry proteins. In at least particular cases, a mutated version of a portion of the PoMV is utilized, such as a deglycosylated, or amino acid substituted mutant of the spike protein.

### CROSS REFERENCE TO RELATED APPLICATIONS

This application claims priority to provisional application U.S. Ser. No. 63/200,313 filed Mar. 1, 2021, which is incorporated herein by reference in its entirety.

### FIELD OF THE INVENTION

This invention relates generally to a novel porcine morbillivirus and related diagnostics, methods of production, and immunogenic compositions that protect mammals from diseases caused by porcine morbillivirus.

### BACKGROUND OF THE INVENTION

Paramyxoviridae encompasses a group of large (300-500 nm in diameter), enveloped, pleomorphic (mostly spherical) viruses with negative-sense, non-segmented RNA genomes of 14.6 to 20.1 kb. The family is composed of four subfamilies and seventeen genera with more than 70 species and contains globally significant viral pathogens affecting human and numerous animal species. Currently, the genus Morbillivirus within subfamily Paramyxovirinae are highly contagious pathogens and include measles virus (MeV), rinderpest virus (RPV), peste des petits rumi-nantsvirus (PPRV), canine distemper virus (CDV), phocine distempervirus (PDV), dolphin morbillivirus (DMV), and feline morbillivirus (FeMV).

Morbillivirus genomes encode six structural proteins in the following order: nucleocapsid (N) protein, phosphoprotein (P), matrix (M), hemagglutinin (H), fusion (F), and large polymerase (L) proteins (2). Two nonstructural proteins, C and V, are expressed from the P open reading frame. These nonstructural proteins are thought to interfere with the innate immune response through blocking IFN responses and limiting production of dsRNA in at least a subset of members of the family Paramyxoviridae.

The two morbillivirus envelope glycoproteins (H & F) encompass a so-called virus fusion-membrane apparatus: the attachment (H) and the fusion (F). The attachment glycoprotein is required for the initial binding step to the host cell receptor, whereas the F glycoprotein mediates the fusion event between the viral and host cell membranes. F glycoprotein also can fuse adjacent cell membranes, forming syncytia when cells expressing H and F interact with cells expressing the receptors. Since the morbillivirus coat consists of hetero-oligomers of the H and F glycoproteins that are organized into tetramers and trimers, respectively, single expression of antigens could alter the overall immunogenicity of the protein. The globular head is the main antigenic determinant of the H protein. Either H- or F-specific antibodies can induce protective titers of neutralizing antibodies,

Morbilliviruses cause moderate to severe respiratory and gastrointestinal disease and profound immune suppression in their respective hosts. Morbillivirus host species experience a similar pathogenesis with infection occurring via inhalation, direct contact with body fluids, through fomites or vertical transmission. Carnivore morbilliviruses readily invade the central nervous system (CNS) and all morbilliviruses produce inclusion bodies containing nucleocapsid-like structures.

Paramyxoviruses that infect swine include porcine rubulavirus, Menangle virus, Nipah virus, and porcine parainfluenza virus. There are reports of less well characterized paramyxoviruses associated with central nervous and respiratory disease in pigs from the United States, Canada, Japan, and Israel. However, none of these viruses are classified in the genus Morbillivirus.

Swine fetuses have been identified to present various forms of neonatal mortality. These fetuses were free of several viruses known to cause neonatal mortality in swine including porcine circovirus 2 (PCV2), porcine circovirus 3 (PCV3), porcine reproductive and respiratory syndrome virus (PRRSV), porcine parvovirus type 1 (PPV1), and Leptospira sp. To date, a morbillivirus in swine has not been identified.

Thus, there is a need to identify unknown viruses that cause disease in livestock, such as swine. Additionally, there is a need to develop a vaccine capable of protecting livestock from identified viruses.

### BRIEF SUMMARY OF THE INVENTION

The whole genome of a novel porcine morbillivirus (PoMV) has been identified and sequenced via metagenomic sequencing and in situ hybridization. The novel morbillivirus has been determined to cause fetal death, encephalitis, placentitis, and other clinical symptoms in swine, though infection in other mammalian species is contemplated. PoMV is a new member in the genus Morbillivirus, subfamily Orthoparamyxovirunae and family Paramyxoviridae. The virus was initially detected in porcine fetal cerebellum, bronchi, bronchioles, spleen, kidney, and liver using an RNA in situ hybridization assay. This finding provides a fundamental basis for future virus isolation, the development of diagnostic tools, and the development of an effective vaccine.

In one aspect, the present invention involves an immunogenic composition comprising heterologous PoMV epitope(s) sequences from PoMV polypeptides designed to elicit an anti-PoMV immune response. Examples of PoMV polypeptides include a nucleocapsid (N) protein (SEQ ID NO: 45), phosphoprotein (P) (SEQ ID NO: 46), C protein, V protein, matrix (M) (SEQ ID NO: 47), fusion (F) (SEQ ID NO: 48), hemagglutinin (H) (SEQ ID NO: 49), and large polymerase (L) proteins (SEQ ID NO: 50). In some embodiments, the present invention is directed to an immunogenic composition for protecting a mammal (e.g, pig, human, chickens, cows, livestock, and the like) against morbillivirus comprising an isolated porcine morbillivirus (PoMV) polypeptide and an expression vector.

In another aspect, a nucleic acid sequence encoding an isolated porcine morbillivirus (PoMV) polypeptide of porcine morbillivirus (PoMV) of bases 1-15661 is disclosed (See SEQ ID NO: 44). In some embodiments, the nucleic acid sequence is operably linked to heterologous targeting, signaling, termination or promoter sequences. In other embodiments, said nucleic acid sequence encodes an amino acid sequence comprising one or more of SEQ ID NOs: 45, 46, 47, 48, 49, and/or 50. In some embodiments, the PoMV polypeptide comprises one or more amino acid substitutions so that said PoMV polypeptide is not naturally occurring. In some embodiments, said substitution occurs at an N-glycosylation site comprising an amino acid deletion and/or an amino acid substitution.

In some embodiments, a method of detecting the presence of at least one antibody directed against porcine morbillivirus (PoMV) in a sample is disclosed. The method includes contacting the sample with a PoMV polypeptide under conditions that allow the formation of an antigen-antibody complex between said PoMV polypeptide and a PoMV antibody present in the sample. In the preferred embodiment, the PoMV peptide comprises the amino acid sequence of SEQ ID NOs: 45, 48, and/or 49. In other embodiments, the PoMV peptide comprises the amino acid sequence of SEQ ID NOs: 46, 47, and/or 50. In other embodiments, additional methods of detecting the antigen-antibody complex are also described. Said methods comprises providing a well of a microtiter plate, wherein the well includes the PoMV polypeptide. A sample is introduced into the well under conditions that allow the formation of the antigen-antibody complex. Next, an enzyme-labeled antibody directed against the sample immunoglobulin is then introduced into the well. Finally, the substrate is added to the well, resulting in detection of the substrate and the presence of PoMV antibody in the sample.

While multiple embodiments are disclosed, still other embodiments of the present invention will become apparent to those skilled in the art from the following detailed description, which shows and describes illustrative embodiments of the invention. Accordingly, the figures and detailed description are to be regarded as illustrative in nature and not restrictive.

### BRIEF DESCRIPTION OF THE DRAWINGS

The following drawings form part of the specification and are included to further demonstrate certain embodiments or various aspects of the invention. In some instances, embodiments of the invention can be best understood by referring to the accompanying drawings in combination with the detailed description presented herein. The description and accompanying drawings may highlight a certain specific example, or a certain aspect of the invention. However, one skilled in the art will understand that portions of the example or aspect may be used in combination with other examples or aspects of the invention.

FIG. 1 is a phylogenetic analysis of the whole PoMV genome sequence. The phylogenic tree was constructed using the maximum likelihood method with booststrap values calculated from 500 trees and rooted on midpoint. The scale bars indicate the branch length that corresponds to 0.1 substitutions per site. Abbreviations: WtlPV, Wenling triplecross lizardfish paramyxovirus; WtPV, Wenling tonguesole paramyxovirus; WhPV, Wenling hoplichthys paramyxovirus; WpssPV, Wenzhou pacific spadenose shark paramyxovirus.

FIG. 2 is a phylogenetic analysis of the L amino acid sequence for the PoMV genome. The tree was constructed by maximum likelihood method with booststrap values calculated from 500 trees and rooted on midpoint. The scale bars indicate the branch length that corresponds to 0.1 substitutions per site. Abbreviations: WtlPV, Wenling triplecross lizardfish paramyxovirus; WtPV, Wenling tonguesole paramyxovirus; WhPV, Wenling hoplichthys paramyxovirus; WpssPV, Wenzhou pacific spadenose shark paramyxovirus.

FIG. 3A is a Histologic section stained by H&E of cerebrum from Fetus A with neuronal necrosis (arrowheads), mineralization, and viral inclusions (arrows) in a neuron and glial cell. FIG. 3B shoes Cerebellum of Fetus A with extensive detection of PoMV by ISH (red). FIG. 3C shows Cerebrum of Fetus A with ISH labeling of neurons (arrowheads) and axon (arrow). FIG. 3D shows Cerebrum of Fetus B with multiple viral inclusion (arrows) in neurons and satellitosis (inset). FIG. 3E Extensive detection of PoMV by ISH in the cerebrum of Fetus B. Arrowheads demark the white and grey matter border. FIG. 3F shows detection of PoMV by ISH in the spleen of Fetus C. FIG. 3G depicts detection of PoMV by ISH in the allantoic epithelium (arrowhead) and leukocytes within subjacent stroma (inset) of the placenta from Litter D. FIG. 3H depicts detection of PoMV by ISH in the endothelium (arrowhead) of a renal vessel (vessel lumen; arrowhead) of a fetus from Litter D. FIG. 3I depicts detection of PoMV by ISH in conducting airways (arrowheads) and alveolar septa in the lung of fetus from Litter E. FIG. 3J shows detection of PoMV by ISH in the allantoic connective tissue and infiltration of leukocytes (arrowhead) of the placenta from Litter F.

FIG. 4 is a multiple sequence alignment of the 3′ Leader and 5′ Trailer sequences of PoMV and other morbilliviruses. The conserved nucleotides are marked by asterisks.

FIG. 5 is a multiple alignment of N proteins (1-80 amino acids) of PoMV and other morbilliviruses. The conserved MA(S,T)L motif in morbilliviruses and the two leucine-rich motifs at the amino acid positions 4-11 and 70-77 are marked in open boxes.

### DETAILED DESCRIPTION OF THE INVENTION

In the present invention, a previously unidentified virus was isolated and characterized using histopathology, metagenomic sequencing and RNA in situ hybridization. The virus was first identified in porcine fetuses and was associated with a variable degree of fetal mortality. The porcine subjects were tested and found to be free from (PCV2), porcine circovirus 3 (PCV3), porcine reproductive and respiratory syndrome virus (PRRSV), porcine parvovirus type 1 (PPV1), and Leptospira sp. After extensive testing, the present inventors were able to isolate the novel morbillivirus from a commercial swine herd and subsequently identify the virus as the putative cause of a reproductive disease characterized by fetal mummification, encephalitis, and placentitis. The present inventors refer to the novel paramyxovirus as porcine morbillivirus (PoMV).

The complete genome sequence of PoMV is disclosed in detail below. In embodiments, said genome may be used to further characterize the novel virus, develop immunogenic compositions (e.g., protein vaccines, RNA vaccines, and DNA vaccines), identify novel cell surface targets, and the like. Further, the present invention enables the development of compositions and methods for detecting a PoMV genome, PoMV expressed proteins, or fragments thereof.

### Definitions

So that the present invention may be more readily understood, certain terms are first defined. Unless defined otherwise, all technical and scientific terms used herein have the same meaning as commonly understood by one of ordinary skill in the art to which embodiments of the invention pertain. Many methods and materials similar, modified, or equivalent to those described herein can be used in the practice of the embodiments of the present invention without undue experimentation, the preferred materials and methods are described herein. In describing and claiming the embodiments of the present invention, the following terminology will be used in accordance with the definitions set out below.

Unless otherwise noted, technical terms are used according to conventional usage. Definitions of common terms in molecular biology may be found in Benjamin Lewin, Genes X, published by Jones & Bartlett Publishers, 2009; and Meyers et al. (eds.), The Encyclopedia of Cell Biology and Molecular Medicine, published by Wiley-VCH in 16 volumes, 2008; and other similar references. Units, prefixes, and symbols may be denoted in their SI accepted form. Unless otherwise indicated, nucleic acids are written left to right in 5′ to 3′ orientation; amino acid sequences are written left to right in amino to carboxy orientation, respectively. Numeric ranges are inclusive of the numbers defining the range and include each integer within the defined range. Amino acids may be referred to herein by either their commonly known three letter symbols or by the one-letter symbols recommended by the IUPAC-IUB Biochemical nomenclature Commission. Nucleotides, likewise, may be referred to by their commonly accepted single-letter codes.

Numeric ranges recited within the specification, including ranges of “greater than,” “at least,” or “less than” a numeric value, are inclusive of the numbers defining the range and include each integer within the defined range.

The singular terms “a”, “an”, and “the” include plural referents unless context clearly indicates otherwise. Similarly, the word “or” is intended to include “and” unless the context clearly indicate otherwise. The word “or” means any one member of a particular list and also includes any combination of members of that list.

“Optional” or “optionally” means that the subsequently described event or circumstance can or cannot occur, and that the description includes instances where the event of circumstance occurs and instances where is does not. For example, the phrase “optionally a signal peptide” means that the signal peptide may or may not be included.

The term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problems or complications commensurate with a reasonable benefit/risk ratio. The term “carrier” means a compound, composition, substance, or structure that, when in combination with a compound or composition, aids or facilitates preparation, storage, administration, delivery, effectiveness, selectivity, or any other feature of the compound or composition for its intended use or purpose. For example, a carrier can be selected to minimize any degradation of the active ingredient and to minimize any adverse side effects in the subject.

As used herein, “adjuvant” means a vehicle used to enhance antigenicity. In some embodiments, an adjuvant can include a suspension of minerals (alum, aluminum hydroxide, or phosphate) on which antigen is adsorbed; or water-in-oil emulsion, for example, in which antigen solution is emulsified in mineral oil (Freund incomplete adjuvant), sometimes with the inclusion of killed mycobacteria (Freund's complete adjuvant) to further enhance antigenicity (inhibits degradation of antigen and/or causes influx of macrophages). Immunostimulatory oligonucleotides (such as those including a CpG motif) can also be used as adjuvants. Adjuvants include biological molecules (a “biological adjuvant”), such as costimulatory molecules. Non-limiting Exemplary adjuvants include Th17 adjuvants (e.g. IL-17), or IL-IL-2, RANTES, GM-CSF, and TNF-α, IFN¬y, G-CSF, LFA-3, CD72, B7-1, B7-2, OX-40 L, 4-lBBL and toll-like receptor (TLR) agonists, such as TLR-9 agonists. In some embodiments, the ADJUPLEX™ (Advanced BioAdjuvants) can be used with any of the immunogens of the present invention to elicit an immune response producing bnAbs. The person of ordinary skill in the art is familiar with adjuvants (see, e.g., Singh (ed.) Vaccine Adjuvants and Delivery Systems. Wiley-Interscience, 2007). Adjuvants can be used in combination with the disclosed immunogens.

As used herein, the term “administration” refers to the introduction of a composition into a subject by a chosen route. Administration can be local or systemic. The compositions utilized in the methods described herein can be administered, for example, intramuscularly, intravenously, intradermally, percutaneously, intraarterially, intraperitoneally, intralesionally, intracranially, intraarticularly, intraprostatically, intrapleurally, intratracheally, intranasally, intravitreally, intravaginally, intrarectally, topically, intratumorally, peritoneally, subcutaneously, subconjunctivally, intravesicularlly, mucosally, intrapericardially, intraocularly, orally, topically, locally, by inhalation, by injection, by infusion, by continuous infusion, by localized perfusion bathing target cells directly, by catheter, by lavage, by gavage, in cremes, or in lipid compositions. The preferred method of administration can vary depending on various factors (e.g., the components of the composition being administered and the severity of the condition being treated). For example, if the chosen route is intravenous, the composition (such as a composition including a disclosed immunogen) is administered by introducing the composition into a vein of the subject.

The terms “peptide,” “protein,” and “polypeptide” are used interchangeably to refer to a natural or synthetic molecule comprising two or more amino acids linked by the carboxyl group of one amino acid to the alpha amino group of another.

The term “protein domain” refers to a portion of a protein, portions of a protein, or an entire protein showing structural integrity; this determination may be based on amino acid composition of a portion of a protein, portions of a protein, or the entire protein.

As used herein, “nucleic acid” refers to a polymer composed of nucleotide units (ribonucleotides, deoxyribonucleotides, related naturally occurring structural variants, and synthetic non-naturally occurring analogs thereof) linked via phosphodiester bonds, related naturally occurring structural variants, and synthetic non-naturally occurring analogs thereof. It will be understood that when a nucleotide sequence is represented by a DNA sequence (i.e., A, T, G, C), this also includes an RNA sequence (i.e., A, U, G, C) in which “U” replaces “T.”

“Nucleotide” includes, but is not limited to, a monomer that includes a base linked to a sugar, such as a pyrimidine, purine or synthetic analogs thereof, or a base linked to an amino acid, as in a peptide nucleic acid (PNA). A nucleotide is one monomer in a polynucleotide. A nucleotide sequence refers to the sequence of bases in a polynucleotide.

The term “variant” refers to an amino acid sequence having conservative amino acid substitutions, non-conservative amino acid substitutions (i.e. a degenerate variant), or a peptide having 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to the recited sequence. The term “conservatively modified variants” applies to both amino acid and nucleic acid sequences. With respect to particular nucleic acid sequences, a conservatively modified variant refers to those nucleic acids, which encode identical or conservatively modified variants of the amino acid sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations” and represent one species of conservatively modified variation. Every nucleic acid sequence herein that encodes a polypeptide also, by reference to the genetic code, describes every possible silent variation of the nucleic acid. One of ordinary skill will recognize that each codon in a nucleic acid (except AUG, which is ordinarily the only codon for methionine; and UGG, which is ordinarily the only codon for tryptophan) can be modified to yield a functionally identical molecule. Accordingly, each silent variation of a nucleic acid, which encodes a polypeptide of the present invention is implicit in each described polypeptide sequence and is within the scope of the present invention.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Thus, any number of amino acid residues selected from the group of integers consisting of from 1 to 15 can be so altered. Thus, for example, 1, 2, 3, 4, 5, 7, or 10 alterations can be made. Conservatively modified variants typically provide similar biological activity as the unmodified polypeptide sequence from which they are derived. For example, generation of immune response, substrate specificity, enzyme activity, or ligand/receptor binding is generally at least 30%, 40%, 50%, 60%, 70%, 80%, or 90% of the native protein for its native substrate. Conservative substitution tables providing functionally similar amino acids, which are well known in the art.

The following six groups each contain amino acids that are conservative substitutions for one another:
Alanine (A), Serine (S), Threonine (T);
Aspartic acid (D), Glutamic acid (E);
Asparagine (N), Glutamine (Q);
Arginine (R), Lysine (K);
Isoleucine (I), Leucine (L), Methionine (M), Valine (V); and
Phenylalanine (F), Tyrosine (Y), Tryptophan (W).
   See also, Creighton (1984) Proteins W.H. Freeman and Company. Further, the term “amino acid substitutions” means the replacement of one amino acid in a polypeptide with a different amino acid or with no amino acid (i.e., a deletion).

The term “percent (%>) sequence identity” or “homology” is defined as the percentage of nucleotides or amino acids in a candidate sequence that are identical with the nucleotides or amino acids in a reference nucleic acid sequence, after aligning the sequences and introducing gaps, if necessary, to achieve the maximum percent sequence identity. Alignment for purposes of determining percent sequence identity can be achieved in various ways that are within the skill in the art, for instance, using publicly available computer software such as BLAST, BLAST-2, ALIGN, ALIGN-2 or Megalign (DNASTAR) software. Appropriate parameters for measuring alignment, including any algorithms needed to achieve maximal alignment over the full-length of the sequences being compared can be determined by known methods. For purposes herein, the % sequence identity of a given nucleotides or amino acids sequence C to, with, or against a given nucleic acid sequence D (which can alternatively be phrased as a given sequence C that has or comprises a certain % sequence identity to, with, or against a given sequence D) is calculated as follows: 100 times the fraction W/Z, where W is the number of nucleotides or amino acids scored as identical matches by the sequence alignment program in that program's alignment of C and D, and where Z is the total number of nucleotides or amino acids in D. It will be appreciated that where the length of sequence C is not equal to the length of sequence D, the % sequence identity of C to D will not equal the % sequence identity of D to C.

As used herein, “contacting” refers to the placement in direct physical association; includes both in solid and liquid form, which can take place either in vivo or in vitro. Contacting includes contact between one molecule and another molecule, for example the amino acid on the surface of one polypeptide, such as a peptide, that contacts another polypeptide. Contacting can also include contacting a cell for example by placing a polypeptide in direct physical association with a cell.

The term “immunogenicity” or “immunogenic” relates to the ability of a substance to stimulate or elicit an immune response. Immunogenicity is measured, for example, by determining the presence of antibodies specific for the substance. The presence of antibodies is detected by methods known in the art, for example using an ELISA, EIA, or Western Blot assay.

As used herein an “immunogenic composition” refers to a composition comprising an immunogenic polypeptide, or a nucleic acid molecule or vector encoding an immunogenic polypeptide that induces a measurable cytotoxic T lymphocyte (CTL) response against the immunogenic polypeptide or induces a measurable B cell response (such as production of antibodies) against the immunogenic polypeptide. In one example, an “immunogenic composition” is a composition that includes a disclosed recombinant or synthesized PoMV protein or immunogenic fragment thereof, that induces a measurable CTL response against an PoMV virus or induces a measurable B cell response (such as production of antibodies) against PoMV. It further refers to isolated nucleic acids encoding an antigen, such as a nucleic acid that can be used to express the antigen (and thus be used to elicit an immune response against this peptide). For in vitro use, an immunogenic composition may comprise or consist of the isolated protein or nucleic acid molecule encoding the protein. For in vivo use, the immunogenic composition will typically include the protein or nucleic acid molecule in a pharmaceutically acceptable carrier and may also include other agents, such as an adjuvant. Any particular protein or immunogenic fragment thereof, or a nucleic acid encoding the protein, can be readily tested for its ability to induce a CTL or B cell response by art-recognized assays. Immunogenic compositions can include adjuvants, which are well known to one of skill in the art.

As used herein, “immunogenic polypeptide” refers to a polypeptide which comprises an allele-specific motif, an epitope or other sequence such that the polypeptide will bind an MHC molecule and induce an immune response, such as a cytotoxic T lymphocyte (“CTL”) response, and/or a B cell response (for example, antibody production), and/or a T-helper lymphocyte response against the antigen from which the immunogenic polypeptide is derived. The term “antigen presentation” means the expression of antigen on the surface of a cell in association with major histocompatibility complex class I or class II molecules (MHC-I or MHC-II) of animals or with the HLA-I and HLA-II of humans.

An “isolated” biological component (such as a protein, for example a disclosed immunogen or nucleic acid encoding such an antigen) has been substantially separated or purified away from other biological components, such as other biological components in which the component naturally occurs, such as other chromosomal and extrachromosomal DNA, RNA, and proteins. Proteins, peptides, and nucleic acids that have been “isolated” include proteins purified by standard purification methods. The term also embraces proteins or peptides prepared by recombinant expression in a host cell as well as chemically synthesized proteins, peptides, and nucleic acid molecules. Isolated does not require absolute purity, and can include protein, peptide, or nucleic acid molecules that are at least 50% isolated, such as at least 75%, 80%, 90%, 95%, 98%, 99%, or even 99.9% isolated.

As used herein, “antibody” means an immunoglobulin, antigen-binding fragment, or derivative thereof, which specifically binds and recognizes an analyte (antigen), an antigenic fragment thereof, or a dimer or multimer of the antigen. The term “antibody” is used herein in the broadest sense and encompasses various structures, including but not limited to monoclonal antibodies, polyclonal antibodies, multi-specific antibodies (e.g., bi-specific antibodies), and antibody fragments, so long as they exhibit the desired antigen-binding activity.

Non-limiting examples of antibodies include, for example, intact immunoglobulins and variants and fragments thereof known in the art that retain binding affinity for the antigen. Examples of antibody fragments include but are not limited to Fv, Fab, Fab′, Fab′-SH, F(ab′)₂; diabodies; linear antibodies; single-chain antibody molecules (e.g. scFv); and multi-specific antibodies formed from antibody fragments. Antibody fragments include antigen binding fragments either produced by the modification of whole antibodies or those synthesized de novo using recombinant DNA methodologies (see, e.g., Kontermann and Dubel (Ed), Antibody Engineering, Vols. 1-2, 2nd Ed., Springer Press, 2010).

Typically, a naturally occurring immunoglobulin has heavy (H) chains and light (L) chains interconnected by disulfide bonds. Immunoglobulin genes include the kappa, lambda, alpha, gamma, delta, epsilon, and mu constant region genes, as well as the myriad immunoglobulin variable domain genes. There are two types of light chain, lambda (λ) and kappa (κ). There are five main heavy chain classes (or isotypes) which determine the functional activity of an antibody molecule: IgM, IgD, IgG, IgA and IgE.

Light and heavy chain variable regions contain a “framework” region interrupted by three hypervariable regions, also called “complementarity-determining regions” or “CDRs” (see, e.g., Kabat et al., Sequences of Proteins of Immunological Interest, U.S. Department of Health and Human Services, 1991). The sequences of the framework regions of different light or heavy chains are relatively conserved within a species. The framework region of an antibody, that is the combined framework regions of the constituent light and heavy chains, serves to position and align the CDRs in three-dimensional space. The CDRs are primarily responsible for binding to an epitope of an antigen.

A “monoclonal antibody” is an antibody produced by a single clone of B-lymphocytes or by a cell into which nucleic acid encoding the light and heavy chains of a single antibody have been transfected, or a progeny thereof. Monoclonal antibodies are produced by methods known to those of skill in the art, for instance by making hybrid antibody-forming cells from a fusion of myeloma cells with immune spleen cells. These fused cells and their progeny are termed “hybridomas.” In some examples monoclonal antibodies are isolated from a subject. Monoclonal antibodies can have conservative amino acid substitutions which have substantially no effect on antigen binding or other immunoglobulin functions. (See, for example, Harlow & Lane, Antibodies, A Laboratory Manual, 2nd ed. Cold Spring Harbor Publications, New York (2013).)

As used herein, the term “antigen” refers to a compound, composition or substance that can stimulate the production of antibodies or a T cell response in an animal, including compositions that are injected or absorbed into an animal. An antigen reacts with the products of specific humoral or cellular immunity, including those induced by heterologous or synthesized antigens, such as PoMV antigens. Examples of antigens include, but are not limited to, polypeptides, peptides, lipids, polysaccharides, combinations thereof (such as glycopeptides) and nucleic acids containing antigenic determinants, such as those recognized by an immune cell. Antigens include peptides derived from PoMV. An antigen can include one or more epitopes.

A “neutralizing antibody” refers to an antibody which reduces the infectious titer of an infectious agent by binding to a specific antigen on the infectious agent, PoMV. A “broadly neutralizing antibody” is an antibody that binds to and inhibits the function of related antigens, such as antigens that share at least 65%, 75%, 85%, 90%, 95%, 96%, 97%, 98% or 99% identity antigenic surface of antigen. Regarding an antigen from a pathogen, such as a virus, the antibody can bind to and inhibit the function of an antigen from more than one class and/or subclass of the pathogen.

An “epitope” is an antigenic determinant. These are particular chemical groups or peptide sequences on a molecule that are antigenic, such that they elicit a specific immune response, for example, an epitope is the region of an antigen to which B and/or T cells respond. An antibody can bind to a particular antigenic epitope, such as an epitope on a PoMV antigen.

Epitopes can be classified as B-cell type, T-cell type or both B cell and T cell type, depending on the type of immune response they elicit. The definition of B cell or T cell peptide epitope is not unequivocal; for example, a peptide epitope can induce antibody production while the epitope can also possess a region that enables binding to the human HLA molecule, rendering it accessible to CTLs, hence a dual B cell and T cell classification for that particular epitope. “CTL”, “killer T cells” or “cytotoxic T cells” is a group of differentiated T cells that recognize and lyse target cells bearing a specific foreign antigen that function in defense against viral infection and cancer cells. “T helper cell” or “Th” is any of the T cells that when stimulated by a specific antigen release cytokines that promote the activation and function of B cells and killer T cells.

As used herein “expression” refers to transcription or translation of a nucleic acid sequence. For example, a gene is expressed when its DNA is transcribed into an RNA or RNA fragment, which in some examples is processed to become mRNA. A gene may also be expressed when its mRNA is translated into an amino acid sequence, such as a protein or a protein fragment. In a particular example, a heterologous gene is expressed when it is transcribed into an RNA.

In another example, a heterologous gene is expressed when its RNA is translated into an amino acid sequence. The term “expression” is used herein to denote either transcription or translation. Regulation of expression can include controls on transcription, translation, RNA transport and processing, degradation of intermediary molecules such as mRNA, or through activation, inactivation, compartmentalization, or degradation of specific protein molecules after they are produced.

Expression control sequences refer to nucleic acid sequences that regulate the expression of a heterologous nucleic acid sequence to which it is operatively linked. Expression control sequences are operatively linked to a nucleic acid sequence when the expression control sequences control and regulate the transcription and, as appropriate, translation of the nucleic acid sequence. Thus, expression control sequences can include appropriate promoters, enhancers, transcription terminators, a start codon (ATG) in front of a protein-encoding gene, splicing signal for introns, maintenance of the correct reading frame of that gene to permit proper translation of mRNA and stop codons. The term “control sequences” is intended to include, at a minimum, components whose presence can influence expression, and can also include additional components whose presence is advantageous, for example, leader sequences and fusion partner sequences. Expression control sequences can include a promoter.

A promoter is a minimal sequence sufficient to direct transcription. Also included are those promoter elements which are sufficient to render promoter-dependent gene expression controllable for cell-type specific, tissue-specific, or inducible by external signals or agents; such elements may be located in the 5′ or 3′ regions of the gene. Both constitutive and inducible promoters are included (see for example, Bitter et al., Methods in Enzymology 153:516-544, 1987). For example, when cloning in bacterial systems, inducible promoters such as pL of bacteriophage lambda, plac, ptrp, ptac (ptrp-lac hybrid promoter) and the like may be used. In one embodiment, when cloning in mammalian cell systems, promoters derived from the genome of mammalian cells (such as metallothionein promoter) or from mammalian viruses (such as the retrovirus long terminal repeat; the adenovirus late promoter; the vaccinia virus 7.5K promoter) can be used. Promoters produced by recombinant DNA or synthetic techniques may also be used to provide for transcription of the nucleic acid sequences. A polynucleotide can be inserted into an expression vector that contains a promoter sequence which facilitates the efficient transcription of the inserted genetic sequence of the host. The expression vector typically contains an origin of replication, a promoter, as well as specific nucleic acid sequences that allow phenotypic selection of the transformed cells.

As used herein, “expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., lentiviruses, retroviruses, adenoviruses, and adena-associated viruses) that incorporate the recombinant polynucleotide.

The term “heterologous” as used herein describes a relationship between two or more elements which indicates that the elements are not normally found in proximity to one another in nature. Thus, for example, a polynucleotide sequence is “heterologous to” an organism or a second polynucleotide sequence if it originates from a foreign species, or, if from the same species, is modified from its original form. For example, a promoter operably linked to a heterologous coding sequence refers to a coding sequence from a species different from that from which the promoter was derived, or, if from the same species, a coding sequence which is not naturally associated with the promoter (e.g., a genetically engineered coding sequence or an allele from a different ecotype or variety). An example of a heterologous polypeptide is a polypeptide expressed from a recombinant polynucleotide in a transgenic organism. Heterologous polynucleotides and polypeptides are forms of recombinant molecules. In one specific, non-limiting example, a heterologous nucleic acid molecule encoding a recombinant PoMV antigen or immunogenic fragment thereof, is expressed in a cell, such as a mammalian cell. Methods for introducing a heterologous nucleic acid molecule in a cell or organism are well known in the art, for example transformation with a nucleic acid, including electroporation, lipofection, particle gun acceleration, and homologous recombination.

By “host cell” is meant a cells in which a vector can be propagated and its DNA expressed. The cell may be prokaryotic or eukaryotic. The term also includes any progeny of the subject host cell. It is understood that all progeny may not be identical to the parental cell since there may be mutations that occur during replication. However, such progeny are included when the term “host cell” is used.

As used herein, “immune response” refers to a response of a cell of the immune system, such as a B cell, T cell, or monocyte, to a stimulus. In one embodiment, the response is specific for a particular antigen (an “antigen-specific response”). In one embodiment, an immune response is a T cell response, such as a CD4+ response or a CD8+ response. In another embodiment, the response is a B cell response, and results in the production of specific antibodies. “Priming an immune response” refers to pre-treatment of a subject with an adjuvant to increase the desired immune response to a later administered immunogenic agent. “Enhancing an immune response” refers to co-administration of an adjuvant and an immunogenic agent, wherein the adjuvant increases the desired immune response to the immunogenic agent compared to administration of the immunogenic agent to the subject in the absence of the adjuvant.

As used herein, “immunogen” refers to a protein or a portion thereof that is capable of inducing an immune response in a mammal, such as a mammal infected or at risk of infection with a pathogen. An appropriate immunogenic preparation can contain, for example, a recombinantly expressed or a chemically synthesized polypeptide (e.g. synthesized by cell-free protein synthesis). Administration of an immunogen can lead to protective immunity and/or proactive immunity against a pathogen of interest. In some examples, an immunogen comprises a recombinant or synthesized PoMV antigen or immunogenic fragment thereof, as disclosed herein.

The DNA strand having the same sequence as an mRNA is referred to as the “coding strand;” sequences on the DNA strand having the same sequence as an mRNA transcribed from that DNA and which are located 5′ to the 5′-end of the RNA transcript are referred to as “upstream sequences;” sequences on the DNA strand having the same sequence as the RNA and which are 3′ to the 3′ end of the coding RNA transcript are referred to as “downstream sequences.”

“cDNA” refers to a DNA that is complementary or identical to an mRNA, in either single stranded or double stranded form.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA produced by that gene produces the protein in a cell or other biological system. Both the coding strand, the nucleotide sequence of which is identical to the mRNA sequence and is usually provided in sequence listings, and non-coding strand, used as the template for transcription, of a gene or cDNA can be referred to as encoding the protein or other product of that gene or cDNA Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

As used herein, “operably linked” is a first nucleic acid sequence is operably linked with a second nucleic acid sequence when the first nucleic acid sequence is placed in a functional relationship with the second nucleic acid sequence. For instance, a promoter, such as the CMV promoter, is operably linked to a coding sequence if the promoter affects the transcription or expression of the coding sequence. Generally, operably linked DNA sequences are contiguous and, where necessary to join two protein-coding regions, in the same reading frame.

“Polypeptide modifications” refers to polypeptides and peptides, such as the recombinant or synthesized PoMV proteins disclosed herein can be modified by a variety of chemical techniques to produce derivatives having essentially the same activity as the unmodified peptides, and optionally having other desirable properties. For example, carboxylic acid groups of the protein, whether carboxyl-terminal or side chain, may be provided in the form of a salt of a pharmaceutically acceptable cation or esterified to form a C1-C16 ester, or converted to an amide of formula NR1R2 wherein R1 and R2 are each independently H or C1-C16 alkyl, or combined to form a heterocyclic ring, such as a 5- or 6-membered ring. Amino groups of the peptide, whether amino-terminal or side chain, may be in the form of a pharmaceutically acceptable acid addition salt, such as the HCl, HBr, acetic, benzoic, toluene sulfonic, maleic, tartaric and other organic salts, or may be modified to C1-C16 alkyl or dialkyl amino or further converted to an amide. Hydroxyl groups of the peptide side chains can be converted to C1-C16 alkoxy or to a C1-C16 ester using well-recognized techniques. Phenyl and phenolic rings of the peptide side chains can be substituted with one or more halogen atoms, such as F, Cl, Br or I, or with C1-C16 alkyl, C1-C16 alkoxy, carboxylic acids and esters thereof, or amides of such carboxylic acids. Methylene groups of the peptide side chains can be extended to homologous C2-C4 alkylenes. Thiols can be protected with any one of a number of well-recognized protecting groups, such as acetamide groups. Those skilled in the art will also recognize methods for introducing cyclic structures into the peptides of this disclosure to select and provide conformational constraints to the structure that result in enhanced stability. For example, a C- or N-terminal cysteine can be added to the peptide, so that when oxidized the peptide will contain a disulfide bond, generating a cyclic peptide. Other peptide cyclizing methods include the formation of thioethers and carboxyl- and amino-terminal amides and esters.

“Prime-boost vaccination” is an immunotherapy including administration of a first immunogenic composition (e.g., the primer) followed by administration of a second immunogenic composition (e.g., the booster vaccine) to a subject to induce an immune response. The primer vaccine and/or the booster vaccine include a vector (such as a viral vector, RNA, or DNA vector) expressing the antigen to which the immune response is directed. The booster vaccine is administered to the subject after the primer vaccine; the skilled artisan will understand a suitable time interval between administration of the primer vaccine and the booster vaccine. In some embodiments, the primer vaccine, the booster vaccine, or both primer vaccine and the booster vaccine additionally include an adjuvant. In one non-limiting example, the primer vaccine is a DNA-based vaccine (or other vaccine based on gene delivery), and the booster vaccine is a protein subunit or protein nanoparticle-based vaccine.

As used herein, “recombinant” refers to a nucleic acid that has a sequence that is not naturally occurring or has a sequence that is made by an artificial combination of two otherwise separated segments of sequence. This artificial combination can be accomplished, for example, the artificial manipulation of isolated segments of nucleic acids, for example using genetic engineering techniques. A recombinant protein is one that has a sequence that is not naturally occurring or has a sequence that is made by an artificial combination of two otherwise separated segments of sequence. In several embodiments, a recombinant protein is encoded by a heterologous (for example, recombinant) nucleic acid that has been introduced into a host cell, such as a bacterial or eukaryotic cell. The nucleic acid can be introduced, for example, on an expression vector having signals capable of expressing the protein encoded by the introduced nucleic acid or the nucleic acid can be integrated into the host cell chromosome.

The term “vector” refers to a nucleic acid molecule as introduced into a host cell, thereby producing a transformed host cell. Recombinant DNA vectors are vectors having recombinant DNA. A vector can include nucleic acid sequences that permit it to replicate in a host cell, such as an origin of replication. A vector can also include one or more selectable marker genes and other genetic elements known in the art. Viral vectors are recombinant nucleic acid vectors having at least some nucleic acid sequences derived from one or more viruses. A replication deficient viral vector is a vector that requires complementation of one or more regions of the viral genome required for replication due to a deficiency in at least one replication-essential gene function. For example, such that the viral vector does not replicate in typical host cells, especially those in a human patient that could be infected by the viral vector in the course of a therapeutic method.

“Virus-like particle” (VLP) is a non-replicating, viral shell, derived from any of several viruses. VLPs are generally composed of one or more viral proteins, such as, but not limited to, those proteins referred to as capsid, coat, shell, surface and/or envelope proteins, or particle-forming polypeptides derived from these proteins. VLPs can form spontaneously upon recombinant expression of the protein in an appropriate expression system. Methods for producing particular VLPs are known in the art. The presence of VLPs following recombinant expression of viral proteins can be detected using conventional techniques known in the art, such as by electron microscopy, biophysical characterization, and the like. Further, VLPs can be isolated by known techniques, e.g., density gradient centrifugation and identified by characteristic density banding. See, for example, Baker et al. (1991) Biophys. J. 60:1445-1456; and Hagensee et al. (1994) J. Viral. 68:4503-4505; Vincente, J Invertebr Pathol., 2011; Schneider Ohrum and Ross, Curr. Top. Microbial. Immunol., 354: 53073, 2012).

A “sample (or biological sample) is a biological specimen containing genomic DNA, RNA (including mRNA), protein, or combinations thereof, obtained from a subject. Examples include, but are not limited to, peripheral blood, tissue, cells, urine, saliva, tissue biopsy, fine needle aspirate, surgical specimen, and autopsy material.

### Polynucleotides of the Invention

In some embodiments, a nucleic acid sequence encoding an isolated porcine morbillivirus (PoMV) polypeptide of porcine morbillivirus (PoMV) of bases 1-15661 is disclosed (See SEQ ID NO: 44). In some embodiments, the nucleic acid sequence is operably linked to heterologous targeting, signaling, termination or promoter sequences. In other embodiments, said nucleic acid sequence encodes an amino acid sequence comprising one or more of SEQ ID NOs: 45, 46, 47, 48, 49, and/or 50. In some embodiments, the PoMV polypeptide comprises one or more amino acid substitutions so that said PoMV polypeptide is not naturally occurring. In other embodiments, said substitution affects an N-glycosylation site. In some embodiments, said substitution at said N-glycosylation site comprises an amino acid deletion and/or an amino acid substitution.

In another aspect, the present invention features isolated polynucleotides including a nucleotide sequence that is at least 90% identical (e.g., at least 91%, 92%, 93%, or 94% identical), at least 95% identical (e.g., at least 96%, 97%, 98%, or 99% identical), or 100% identical to all or a portion of the reference sequence, or its complement. The reference sequence is the full-length genome sequence of wild-type PoMV (SEQ ID NO: 44). The isolated nucleotide or polynucleotides of the invention may include at least 5, 6, 7, 8, 9, 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, 10000, 15000, 20000, or 25000 or more contiguous or non-contiguous nucleotides of a reference polynucleotide molecule.

In some embodiments, the isolated polynucleotides of the invention include a nucleotide sequence that is at least 90% identical (e.g., at least 91%, 92%, 93%, or 94% identical), at least 95% identical (e.g., at least 96%, 97%, 98%, or 99% identical), or 100% identical to all or a portion of any one of proteins encoded by the PoMV genome, or its complement. In other embodiments, an isolated nucleic acid molecule comprises a nucleotide sequence selected from a nucleotide sequence encoding the amino acid sequence of porcine morbillivirus nucleocapsid protein, phosphoprotein, matrix protein, fusion protein, hemagglutinin protein, and/or large protein.

In another aspect, the invention provides polynucleotide sequences related to PoMV. The isolated polynucleotides may include a nucleotide sequence that is at least 90% identical (e.g., at least 91%, 92%, 93%, or 94% identical), at least 95% identical (e.g., at least 96%, 97%, 98%, or 99% identical), or 100% identical to all or a portion of any one of the full-length genome sequence of wild-type PoMV or the complement. The isolated polynucleotides of the invention may include at least 5, 6, 7, 8, 9, 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, 10000, 15000, 20000, or more contiguous or non-contiguous nucleotides of the PoMV genome.

In some embodiments, the polynucleotides of the invention may be used as primers that are between 10-100 nucleotides in length, more particularly between 10-30 nucleotides in length (e.g., 10, 11, 12, 13, 14, 15, 16, 17, 18, 19, 20, 21, 22, 23, 24, 25, 26, 27, 28, 29, or 30 nucleotides in length), and can be at least 90% identical (e.g., at least 91%, 92%, 93%, or 94% identical), at least 95% identical (e.g., at least 96%, 97%, 98%, or 99% identical), or 100% identical to any one of sequences in Table 1.

TABLE 1
Porcine Morbillivirus RT-PCT and Quantitative
PCR Primers and Probe

| | SEQ | | | |
|---|---|---|---|---|
| Primer | ID | | | |
| ID | NO:. | Name | Sequence | Use |
| 1 | 1 | MBL-1Fa | 5′-ACCAAACAAAGTTGGGTAAG-3′ | Genome |
| | | | | sequence |
| 2 | 2 | MBLV-900F | 5′-GGGCTAGCAAGCTTTATCCTCAC-3′ | Genome |
| | | | | sequence |
| 3 | 3 | MBLV-988R | 5′-GTTAACTCACCAGCAAACTCATGC-3′ | Genome |
| | | | | sequence |
| 4 | 4 | MBL-2000F | 5′-ATGAATCACAGACGGCAAC-3′ | Genome |
| | | | | sequence |
| 5 | 5 | MBL-2100R | 5′-AACATAGTAGTGCTGTATGTCAGATC-3′ | Genome |
| | | | | sequence |
| 6 | 6 | MBL-3300R | 5′-CATTTGGTGGAATTCCTTGA-3′ | Genome |
| | | | | sequence |
| 7 | 7 | MBL-3200F | 5′-GATAAAGTCAAGCAGCCTGGA-3′ | Genome |
| | | | | sequence |
| 8 | 8 | MBL-4515R | 5′-GTCACTGCTTGGCCCTC-3′ | Genome |
| | | | | sequence |
| 9 | 9 | MBL-4415F | 5′-CCAGGGGCTGTTCAAGATCCTATAAG-3′ | Genome gap |
| | | | | closure |
| 10 | 10 | MBL-5462R | 5′-GCCGGTCACATTAGGCATAAGTTTTATAAC-3′ | Genome gap |
| | | | | closure |
| 11 | 11 | MBL-5400F | 5′-TACAAAATTATGGCACCCG-3′ | Genome |
| | | | | sequence |
| 12 | 12 | MBL-6600R | 5′-CATAAACAGTGTCTGGGTACTCTCT-3′ | Genome |
| | | | | sequence |
| 13 | 13 | MBL-6500F | 5′-TTACTTACATTCATCGCGACTGAT-3′ | Genome |
| | | | | sequence |
| 14 | 14 | MBL-7700R | 5′-CTAGTAAGAGATAAGGCAGGTGATATAA-3′ | Genome |
| | | | | sequence |
| 15 | 15 | MBL-7600F | 5′-CTTATCACATAGCAAATCAGATATATTCCC-3′ | Genome |
| | | | | sequence |
| 16 | 16 | MBL-8800R | 5′-CACCATATTTTCTCAACCCATACA-3′ | Genome |
| | | | | sequence |
| 17 | 17 | MBL-8700F | 5′-CATGCAATAGTGTATTATATCTACAATCC-3′ | Genome |
| | | | | sequence |
| 18 | 18 | MBL-9900R | 5′-TAAGCGAGGGAAAGTGGTTC-3′ | Genome |
| | | | | sequence |
| 19 | 19 | MBL-9800F | 5′-CTCTGGGATCTGATTGACGG-3′ | Genome |
| | | | | sequence |
| 20 | 20 | MBL-11000R | 5′-CTTACAGTCTCATAAAGGTCTATGTCATT-3′ | Genome |
| | | | | sequence |
| 21 | 21 | MBL-10900F | 5′-GTCTCCCAAAATGTCAACTTG-3′ | Genome |
| | | | | sequence |
| 22 | 22 | MBL-12100R | 5′-CTGTTGACTAGCACGTGCCT-3′ | Genome |
| | | | | sequence |
| 23 | 23 | MBL-12000F | 5′-TTAGATTGGGCAAGTGATCC-3′ | Genome |
| | | | | sequence |
| 24 | 24 | MBL-13200R | 5′-TGTTTGGGACTGCCGAAG-3′ | Genome |
| | | | | sequence |
| 25 | 25 | MBL-13100F | 5′-AACACAGTCCTCCATTTACATGT-3′ | Genome |
| | | | | sequence |
| 26 | 26 | MBL-14300R | 5′-CTTCTAAATGCATGGATTTCGTA-3′ | Genome |
| | | | | sequence |
| 27 | 27 | MBL-14200F | 5′-GACGATTTACTATCATTAATGGGATCTAT-3′ | Genome |
| | | | | sequence |
| 28 | 28 | MBL-15665R | 5′-AAACAAAATTGGCTAAGGGAAC-3′ | Genome |
| | | | | sequence |
| 2 | 29 | MBLV-900F | 5′-GGGCTAGCAAGCTTTATCCTCAC-3′ | PoMV |
| | | | | forward |
| | | | | primer |
| 3 | 30 | MBLV-988R | 5′-GTTAACTCACCAGCAAACTCATGC-3′ | PoMV |
| | | | | forward |
| | | | | primer |
| 29 | 31 | MBL-959P | 5′-6-FAM-CAGTGCCGGGTACATAGTTTCTATCCC | PoMV probe |
| | | | AA-TAMSp-3′ | |

In some embodiments, the polynucleotides of the invention include all or a portion of the nucleotide sequence encoding the protein of wild-type PoMV. In some embodiments, the nucleotide sequence encoding all or a portion of the protein can be at least 90% identical (e.g., at least 91%, 92%, 93%, or 94% identical), at least 95% identical (e.g., at least 96%, 97%, 98%, or 99% identical), or 100% identical to the nucleotide sequence encoding the protein of PoMV. The proteins of PoMV (also referred to herein as “PoMV polypeptide(s)”) correspond to the N, PN/C, M, F, H, and/or L proteins as discussed in the Examples. In some embodiments, said polynucleotides (also referred to herein as “nucleic acid sequences”) encode an isolated porcine morbillivirus (PoMV) polypeptide of porcine morbillivirus (PoMV) of bases 1-15661 operably linked to heterologous targeting, signaling, termination or promoter sequences.

### Immunogens, Diagnostics, and Related Methods

In one aspect, the present invention involves a immunogenic composition comprising heterologous PoMV epitope(s) sequences from PoMV polypeptides designed to elicit an anti-PoMV immune response. In embodiments, PoMV polypeptide(s) may comprise a single polypeptide, a complex of polypeptides, a sequence of distinct yet covalently bound polypeptides, and the like. Examples of PoMV polypeptides include nucleocapsid (N) protein, phosphoprotein (P), matrix (M), hemagglutinin (H), fusion (F), and large polymerase (L) proteins (2).

In some embodiments, the present invention is directed to an immunogenic composition for protecting a mammal (e.g, pig, human, chickens, cows, livestock, and the like) against morbillivirus respiratory, central nervous system, and gastrointestinal disease comprising an isolated porcine morbillivirus (PoMV) polypeptide of porcine morbillivirus (PoMV) and a vector (e.g. an expression vector). In other embodiments, the recombinant expression vector is a baculovirus, cosmid, plasmid (e.g., naked or contained in liposomes), virus (e.g., lentiviruses, retroviruses, adenoviruses, and adena-associated viruses) that incorporate the recombinant polynucleotide, or the like. In other embodiments, the immunogenic composition comprises an adjuvant, or a comparable element known in the art that induces a stronger immune response in subjects receiving the vaccine.

In another embodiment, an immunogen of the present invention comprises a PoMV polypeptide. In some embodiments, the PoMV polypeptide comprises a fragment of amino acids including at least 1, 2, 3, 4, 5, 10, 15, 20, 30, 40, 50, 100, or 200 amino acids of the PoMV polypeptide (also referred to herein as “isolated PoMV polypeptide”). In embodiments, PoMV polypeptide comprises a nucleocapsid (N) protein (SEQ ID NO: 45), phosphoprotein (P) (SEQ ID NO: 46), C protein, V protein, matrix (M) (SEQ ID NO: 47), fusion (F) (SEQ ID NO: 48), hemagglutinin (H) (SEQ ID NO: 49), and large polymerase (L) proteins (SEQ ID NO: 50). In embodiments, the two nonstructural proteins, C and V, are expressed from the P open reading frame. In embodiments, proteins N, P, H, F, and/or L M, F, H, and/or L have at least 70%, 73%, 76%, 79%, 82%, 85%, 88%, 91%, 94%, or 97% sequence identity with nucleocapsid (N) protein (SEQ ID NO: 45), phosphoprotein (P) (SEQ ID NO: 46), matrix (M) (SEQ ID NO: 47), hemagglutinin (H) (SEQ ID NO: 49), fusion (F) (SEQ ID NO: 48), and large polymerase (L) proteins (SEQ ID NO: 50), respectively. The immunogenic peptides and polypeptides of the present invention may be synthesized chemically using methods known in the art for synthesis of peptides, peptide multimers and polypeptides. These methods generally rely on the known principles of peptide synthesis. Most conveniently, the procedures can be performed according to known principles of solid phase peptide synthesis.

In embodiments, PoMV polypeptide comprises a fusion-membrane apparatus, (also referred to herein as “PoMV attachment glycoprotein” or “PoMV attachment glycoprotein complex”). In some embodiments, the fusion-membrane apparatus comprises at least two morbillivirus envelope glycoproteins including hemagglutinin (H) (SEQ ID NO: 49) (“H protein”) and fusion (F) (SEQ ID NO: 48) proteins. In other embodiments, the fusion-membrane apparatus comprises more than two morbillivirus envelope glycoproteins, such as 3, 4, or 5 envelope glycoproteins. In some embodiments, hemagglutinin (H) (SEQ ID NO: 49) is required for the initial binding step to the host cell receptor, whereas fusion (F) (SEQ ID NO: 48)(also referred to as “F glycoprotein” or “F protein”) mediates fusion events between viral and host cell membranes. In embodiments, F glycoprotein also can fuse adjacent cell membranes, forming syncytia when cells expressing H and F interact with cells expressing the receptors.

In some embodiments, since the morbillivirus coat consists of hetero-oligomers of the H and F glycoproteins that are organized into tetramers and trimers, respectively, single expression of antigens could alter the overall immunogenicity of the protein. In some embodiments, H protein (SEQ ID NO: 49) comprises a globular head (“GB”) domain and stalk domain. In embodiments, the GB domain is immunodominant of the stalk domain. In some embodiments, the GB domain is the main antigenic determinant of H protein. In some embodiments, either H- or F-specific antibodies can induce protective titers of neutralizing antibodies for protecting a pig against morbillivirus respiratory and gastrointestinal disease.

In some embodiments, the PoMV polypeptide has sequence homology to fusion protein (F), a putative antigenic determinant for PoMV. In some embodiments, the PoMV polypeptide has at least 60%, 70%, 73%, 76%, 79%, 82%, 85%, 88%, 91%, 94%, or 97% sequence identity with fusion (F) (SEQ ID NO: 48) protein. In other embodiments, the PoMV polypeptide has at least 60%, 70%, 73%, 76%, 79%, 82%, 85%, 88%, 91%, 94%, or 97% sequence identity with Hemagglutinin (H) protein (SEQ ID NO: 49), another putative antigenic determinant. In some embodiments, a method for protecting a mammal (e.g, pig, human, chickens, cows, livestock, and the like) against porcine morbillivirus respiratory, central nervous system, and gastrointestinal disease involves administering to the mammal the above-described immunogenic compositions (e.g. SEQ ID NO: 48, SEQ ID NO: 49, and the like). In some embodiments, the composition is administered via a route selected from the group consisting of intravenous, intramuscular, intradermal, subcutaneous, peritoneal, and oral.

In some embodiments, a method is disclosed that aides in the prevention or reduction of one or more clinical symptoms associated with infection with porcine morbillivirus respiratory and gastrointestinal disease, said method including administering the above-described compositions (e.g. polypeptides SEQ ID NO: 48, SEQ ID NO: 49, and the like) to a subject. In some embodiments, the clinical symptoms are selected from the group consisting of encephalitis, placentitis, nonsuppurative myocarditis, coughing, wheezing, dyspnea, polypnea, increased mortality rate, fetal death, aborted fetus, neuronal necrosis, neuronal and glial mineralization, neuronal viral inclusion bodies, lesions of the ganglia, and any combination thereof.

In some embodiments, a method of detecting the presence of at least one antibody directed against porcine morbillivirus (PoMV) in a sample is disclosed. The method includes contacting the sample with a PoMV polypeptide under conditions that allow the formation of an antigen-antibody complex between said PoMV polypeptide and a PoMV antibody present in the sample, wherein the PoMV polypeptide comprises a fragment of at least 1, 2, 3, 4, 5, 8, 10, 15, 10, 30, 40, 50, 60, or 75 amino acids and wherein the PoMV peptide comprises at least 60%, 70%, 73%, 76%, 79%, 82%, 85%, 88%, 91%, 94%, or 97% identity to the amino acid sequence of SEQ ID NOs: 45, 48, and/or 49.

In embodiments, the present inventors also contemplate a method wherein the antigen-antibody complex is detected (e.g., identified in a diagnostic assay). Said detection method may be facilitated by a variety of techniques and probes known in the art. First, the methods comprises providing a well of a microtiter plate, wherein the well includes the PoMV polypeptide. In some embodiments, a mammal sample (e.g. a human sample, a pig sample, a livestock sample, and aquatic mammal sample, or the like) is then introduced into the well under conditions that allow the formation of the antigen-antibody complex. In embodiments, an enzyme-labeled antibody directed against the sample immunoglobulin is then introduced into the well, wherein the enzyme adapted for use with the enzyme-labeled antibody is capable of hydrolyzing a substrate. In some embodiments, the substrate is added to the well, thereby detecting the substrate (e.g., inducing a detection event) and the presence of PoMV antibody in the sample.

In one example, the sample source used to detect the presence of at least one PoMV antibody is serum, purified nucleic acid, whole blood, or biopsies. In some embodiments, the PoMV polypeptide in the well is selected from the group consisting of SEQ ID NO: 45, SEQ ID NO: 46, SEQ ID NO: 47, SEQ ID NO: 49, SEQ ID NO: 48, and SEQ ID NO: 50. In some embodiments, the polypeptide is labeled with a detectable marker. In other embodiments, the above-described antigen-antibody complex is detected via the ELISA, immunofluorescence, by radioimmunological processes (RIA), or their equivalent. In other embodiments, the polypeptide comprises a fragment of at least 10, 15, or 20 amino acids of polypeptide having at least 30%, 40%, 60%, 70%, 73%, 76%, 79%, 82%, 85%, 88%, 91%, 94%, or 97% identity to the amino acid sequence of SEQ ID NO: 45, 46, 47, 48, 49, or 60.

In another aspect, the invention provides vaccine to PoMV. In an embodiment, the vaccine is an attenuated virus, a whole intact virus, a protein subunit, a virus-like particle (VLP), a viral vector, a DNA vaccine, an RNA vaccine, and/or an artificial antigen-presenting cell (aAPC). In some embodiments, the vaccine comprises a subunit vaccine including a nucleocapsid (N) protein (SEQ ID NO: 45), phosphoprotein (P) (SEQ ID NO: 46), C protein, V protein, matrix (M) (SEQ ID NO: 47), fusion (F) (SEQ ID NO: 48), hemagglutinin (H) (SEQ ID NO: 49), and/or a large polymerase (L) protein (SEQ ID NO: 50).

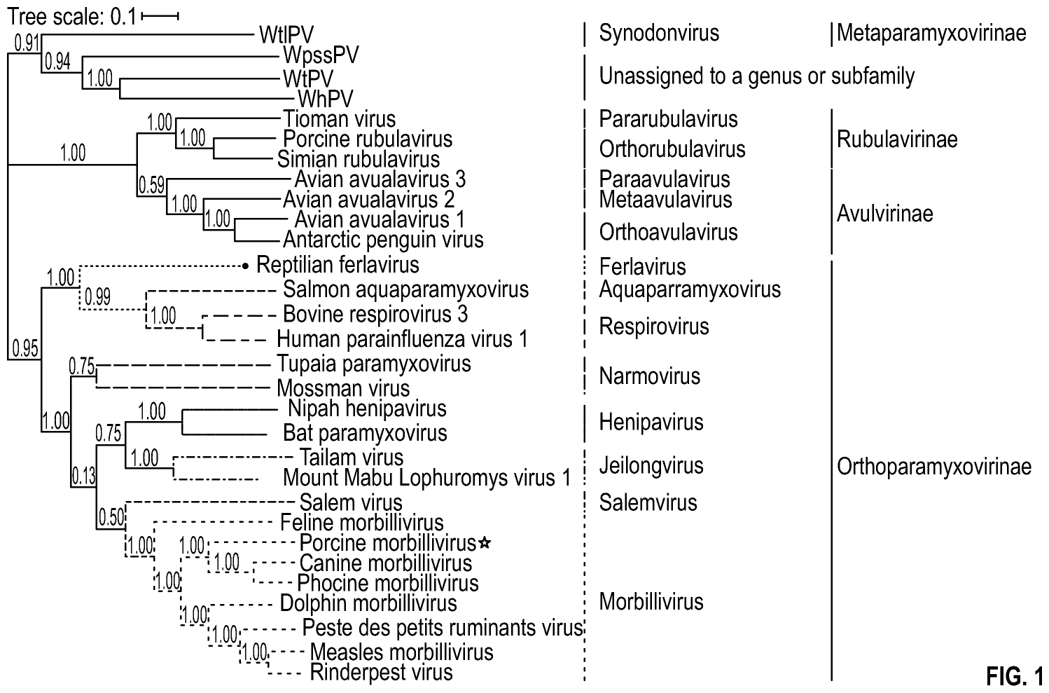

As shown in FIG. 1, PoMV clustered with other morbilliviruses, with high bootstrap supporting a distinct subgroup (FIG. 1). Both phylogenetic analyses also confirmed the findings from the results of pairwise alignment and demonstrated that PoMV was most closely related to CDV and PDV; closely related to CMV, PPRV, MeV, and RPV; and most distantly related to FeMV in the genus Morbillivirus (FIG. 1). Overall, these data further support that PoMV is a previously undescribed member in the genus Morbillivirus, subfamily Orthoparamyxovirinae, and family Paramyxoviridae.

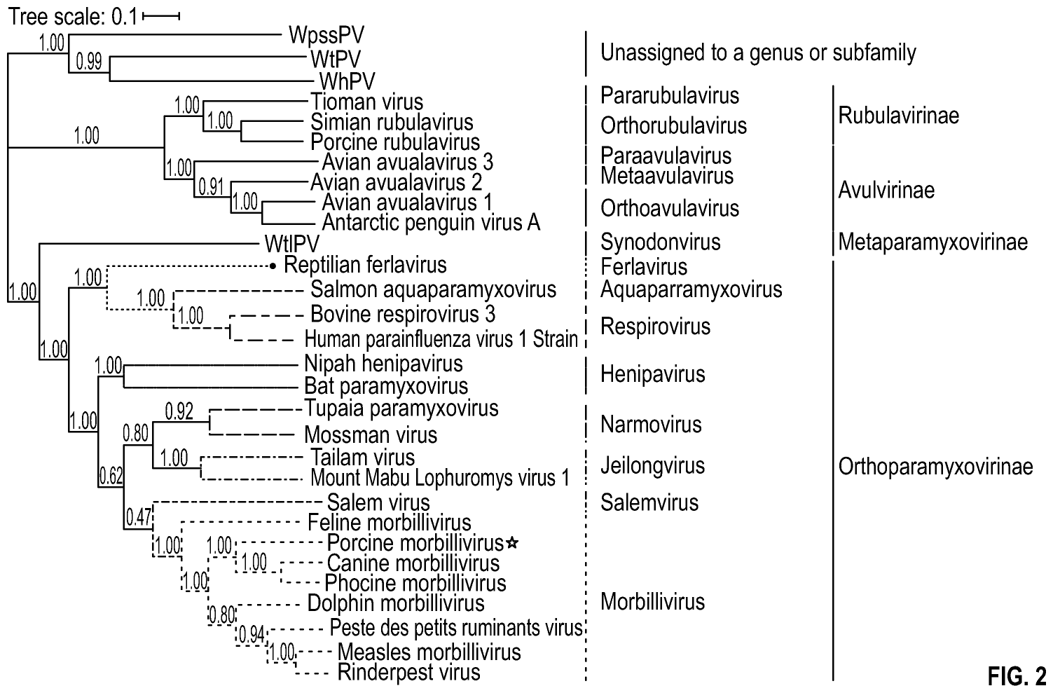

As described above, H protein (SEQ ID NO: 49) comprises a globular head (“GB”) domain and stalk domain. In embodiments, the GB domain is immunodominant of the stalk domain. In some embodiments, the GB domain is the main antigenic determinant of H protein. In some embodiments, either H- or F-specific antibodies can induce protective titers of neutralizing antibodies for protecting a pig against morbillivirus respiratory and gastrointestinal disease. As suggested by the phylogenic analysis shown in FIG. 1 and FIG. 2, PoMV is most closely related to CDV, whose antigenic determinants are similarly identified as NP, P. F and 1-1 proteins (see, J. gen. Virol. 1985, 66, 443-456). Peptide analogs and peptidomimetics are also included within the scope of the invention, as well as salts and esters of the peptides of the invention. A peptide analog according to the present invention may optionally comprise at least one non-natural amino acid and/or at least one blocking group at either the C terminus or N terminus. Salts of the peptides of the invention are physiologically acceptable organic and inorganic salts. The design of appropriate “analogs” may be computer assisted.

The term “peptidomimetic” means that a peptide according to the invention is modified in such a way that it includes at least one non-peptidic bond such as, for example, urea bond, carbamate bond, sulfonamide bond, hydrazine bond, or any other covalent bond. The design of “peptidomimetics” may be computer assisted.

Salts and esters of the peptides of the invention are encompassed within the scope of the invention. Salts of the peptides of the invention are physiologically acceptable organic and inorganic salts. Functional derivatives of the peptides of the invention cover derivatives which may be prepared from the functional groups which occur as side chains on the residues or the N- or C-terminal groups, by means known in the art, and are included in the invention as long as they remain pharmaceutically acceptable, i.e., they do not destroy the activity of the peptide and do not confer toxic properties on compositions containing it. These derivatives may, for example, include aliphatic esters of the carboxyl groups, amides of the carboxyl groups produced by reaction with ammonia or with primary or secondary amines, N-acyl derivatives of free amino groups of the amino acid residues formed by reaction with acyl moieties (e.g., alkanoyl or carbocyclic aroyl groups) or O-acyl derivatives of free hydroxyl group (for example that of seryl or threonyl residues) formed by reaction with acyl moieties.

Also disclosed are polynucleotides comprising nucleic acid sequences encoding the immunogen. For example, the nucleic acid sequences can be operably linked to expression control sequences. Thus, also disclosed are expression vectors for producing the antigens as well as cells containing these polynucleotides and vectors for replicating the polynucleotides and vectors or to produce the proteins and/or VLPs and/or inactivated/attenuated live or killed viruses. Therefore, the disclosed cell can also contain nucleic acid sequences encoding a PoMV protein and/or including a vector comprising the nucleic acid sequences encoding a PoMV protein.

The cell can be a prokaryotic or eukaryotic cell. For example, the cell can be a bacterium, an insect cell, a yeast cell, or a mammalian cell. The cell can be a human cell. Suitable vectors can be routinely selected based on the choice of cell used to produce the VLP. For example, where insect cells are used, suitable vectors include baculoviruses.

Fusion proteins, also known as chimeric proteins, are proteins created through the joining of two or more genes which originally coded for separate proteins. Translation of this fusion gene results in a single polypeptide with function properties derived from each of the original proteins. Recombinant fusion proteins can be created artificially by recombinant DNA.

The functionality of fusion proteins is made possible by the fact that many protein functional domains are modular. In other words, the linear portion of a polypeptide which corresponds to a given domain, such as a tyrosine kinase domain, may be removed from the rest of the protein without destroying its intrinsic enzymatic capability. Thus, any of the herein disclosed functional domains can be used to design a fusion protein.

If the two entities are proteins, often linker (or “spacer”) peptides are also added which make it more likely that the proteins fold independently and behave as expected. Especially in the case where the linkers enable protein purification, linkers in protein or peptide fusions are sometimes engineered with cleavage sites for proteases or chemical agents which enable the liberation of the two separate proteins.

In a non-limiting example, the chimeric polypeptide of the present invention includes chimeras of a PoMV peptide epitope with one of the following, polypeptides: Cholera toxin, Tetanus toxin, Ovalbumin, Tuberculosis heat shock protein, Diphtheria Toxoid, Protein G from respiratory syncytial virus, Outer Membrane Protein from Neisseria meningitides, nucleoprotein of vesicular stomatitis virus, glycoprotein of vesicular stomatitis virus, Plasmodium falciparum Antigen Glutamate-Rich Protein, Merozoite Surface Protein 3 or Viruses envelope protein.

The term “expression vector” and “recombinant expression vector” as used herein refers to a DNA molecule, for example a plasmid or virus, containing a desired and appropriate nucleic acid sequences necessary for the expression of the recombinant peptide epitopes for expression in a particular host cell. As used herein “operably linked” refers to a functional linkage of at least two sequences. Operably linked includes linkage between a promoter and a second sequence, for example an nucleic acid of the present invention, wherein the promoter sequence initiates and mediates transcription of the DNA sequence corresponding to the second sequence.

The regulatory regions necessary for transcription of the peptide epitopes can be provided by the expression vector. The precise nature of the regulatory regions needed for gene expression may vary among vectors and host cells. Generally, a promoter is required which is capable of binding RNA polymerase and promoting the transcription of an operably-associated nucleic acid sequence. Regulatory regions may include those 5′ non-coding sequences involved with initiation of transcription and translation, such as the TATA box, capping sequence, CAAT sequence, and the like. The non-coding region 3′ to the coding sequence may contain transcriptional termination regulatory sequences, such as terminators and polyadenylation sites. A translation initiation codon (ATG) may also be provided.

To clone the nucleic acid sequences into the cloning site of a vector, linkers or adapters providing the appropriate compatible restriction sites are added during, synthesis of the nucleic acids. For example, a desired restriction enzyme site can be introduced into a fragment of DNA by amplification of the DNA by use of PCR with primers containing the desired restriction enzyme site.

An expression construct comprising a peptide epitope sequence operably associated with regulatory regions can be directly introduced into appropriate host cells for expression and production of the multiepitope polypeptide per se or as recombinant immunogen. The expression vectors that may be used include but are not limited to plasmids, cosmids, phage, phagemids, flagellin or modified viruses. Typically, such expression vectors comprise a functional origin of replication for propagation of the vector in an appropriate host cell, one or more restriction endonuclease sites for insertion of the desired gene sequence, and one or more selection markers.

The recombinant polynucleotide construct comprising the expression vector and a immunogenic polypeptide should then be transferred into a bacterial host cell where it can replicate and be expressed. This can be accomplished by methods known in the art. The expression vector is used with a compatible prokaryotic or eukaryotic host cell which may be derived from bacteria, yeast, insects, mammals, and humans.

### Production of Immunogenic Polypeptide

In some embodiments, a process is disclosed for the production of a composition for aiding in the prevention or reduction of one or more clinical symptoms associated with infection by a porcine morbillivirus respiratory and gastrointestinal disease. In embodiments, said method comprises infecting cells with a recombinant expression vector including nucleic acids encoding the PoMV polypeptide of porcine morbillivirus. Next, PoMV polypeptide of PoMV is expressed via a vector (e.g. a recombinant expression vector). In embodiments, the expressed PoMV polypeptide of PoMV is then recovered. In some embodiments, the above described PoMV polypeptide is encoded by a nucleic acid comprising SEQ ID NO: 44, SEQ ID NO: 48, SEQ ID NO: 49, or SEQ ID NO: 50. In other embodiments, the recovered PoMV polypeptide of PoMV is combined with an adjuvant. In still other embodiments, the recovered PoMV polypeptide of PoMV is combined with a pharmaceutically or veterinarily acceptable carrier.

In some embodiments, at least one antigen that is not an isolated PoMV polypeptide of PoMV is used to elicit an immune response. In other embodiments, a method for protecting a pig against infection by porcine morbillivirus respiratory and gastrointestinal disease is contemplated, the method including administering to the pig the above described immunogenic non-PoMV related composition.

Once expressed by the host cell, the immunogenic polypeptide can be separated from undesired components by a number of protein purification methods. One such method uses a polyhistidine tag on the recombinant protein. A polyhistidine-tag consists in at least six histidine (His) residues added to a recombinant protein, often at the N- or C-terminus Polyhistidine-tags are often used for affinity purification of polyhistidine-tagged recombinant proteins that are expressed in E. coli or other prokaryotic expression systems. The bacterial cells are harvested by centrifugation and the resulting cell pellet can be lysed by physical means or with detergents or enzymes such as lysozyme. The raw lysate contains at this stage the recombinant protein among several other proteins derived from the bacteria and are incubated with affinity media such as NTA-agarose, HisPur resin or Talon resin. These affinity media contain bound metal ions, either nickel or cobalt to which the polyhistidine-tag binds with micromolar affinity. The resin is then washed with phosphate buffer to remove proteins that do not specifically interact with the cobalt or nickel ion. The washing efficiency can be improved by the addition of 20 mM imidazole and proteins are then usually eluted with 150-300 mM imidazole. The polyhistidine tag may be subsequently removed using restriction enzymes, endoproteases or exoproteases. Kits for the purification of histidine-tagged proteins can be purchased for example from Qiagen.

Another method is through the production of inclusion bodies, which are inactive aggregates of protein that may form when a recombinant polypeptide is expressed in a prokaryote. While the cDNA may properly code for a translatable mRNA, the protein that results may not fold correctly, or the hydrophobicity of the added peptide epitopes may cause the recombinant polypeptide to become insoluble. Inclusion bodies are easily purified by methods well known in the art. Various procedures for the purification of inclusion bodies are known in the art. In some embodiments the inclusion bodies are recovered from bacterial lysates by centrifugation and are washed with detergents and chelating agents to remove as much bacterial protein as possible from the aggregated recombinant protein. To obtain soluble protein, the washed inclusion bodies are dissolved in denaturing agents and the released protein is then refolded by gradual removal of the denaturing reagents by dilution or dialysis (as described for example in Molecular cloning: a laboratory manual, 3rd edition, Sambrook, J. and Russell, D. W., 2001; CSHL Press).

### Virus Like Particles (VLPs)

The disclosed sequences may be expressed on the surface of a particle to mimic the natural conformation of virions. For example, the antigens may be incorporated into virus-like particles (VLPs). Non-replicating VLPs resemble infectious virus particles in structure and morphology and contain immunologically relevant viral structural proteins. VLPs have been produced from both non-enveloped and enveloped viruses. Envelopes of VLPs are derived from the host cells similar to the way as enveloped viruses obtain their lipid envelopes from their host cells. Therefore, membrane-anchored proteins on the surfaces of enveloped viruses will be expressed in a native-like conformation if they are expressed in a membrane-anchored form.

VLPs involve lipid bilayers and host cell membrane proteins (Song, J. M., et al. J Proteome Res 2011 10:3450-3459). VLPs are produced, for example, by coinfecting insect cells with one or more recombinant baculoviruses co-expressing Ml proteins and the PoMV antigens, culturing the insect cells under physiological conditions, and purifying the VLPs from insect cell culture supernatants.

### Viral Vectors

Viral vector immunogenic compositions (e.g., vaccines) consist of a recombinant virus, the viral vector, often attenuated to reduce its pathogenicity, in which genes encoding viral antigen(s) have been cloned using recombinant DNA techniques. Vector vaccines can either be replicating or non-replicating. Replicating vector vaccines infect cells in which the vaccine antigen is produced as well as more infectious viral vectors able to infect new cells that will then also produce the vaccine antigen. Non-replicating vector vaccines initially enter cells and produce the vaccine antigen, but no new virus particles are formed. Because viral vector vaccines result in endogenous antigen production, both humoral and cellular immune responses are stimulated. One advantage of these viral vector-based vaccines is therefore that a single dose can be sufficient for protection.

Adenoviral vector systems may also be used to form viral vectors. In adenoviral vector systems, adenovirus serotype-5 (Ad5)-based immunotherapeutics that have been repeatedly used in humans to induce robust T cell-mediated immune (CMI) responses, all while maintaining an extensive safety profile. In addition, Ad5 vectors can be reliably manufactured in large quantities and are stable for storage and delivery for outpatient administration. Nonetheless, a major obstacle to the use of first generation Ad5-based vectors is the high frequency of pre-existing anti-adenovirus type 5 neutralizing antibodies. The term “First Generation adenovirus”, as used herein, refers to an Ad that has the early region 1 (E1) deleted. In additional cases, the nonessential early region 3 (E3) may also be deleted.

These antibodies can be present in a potential vaccine due to either prior wild type adenovirus infection and/or induction of adenovirus neutralizing antibodies by repeated injections with Ad5-based vaccines, each resulting in inadequate immune stimulation against the target TAA. First Generation adenovirus vector vaccines express Ad late genes, albeit at a decreased level and over a longer time period than wild-type Ad virus (Nevins, et al. Cell 26/213-220 (1981); Gaynor, et al. Cell 33/683-693 (1983); Yang, et al. J Virol 70/7209-7212 (1996)). When using First Generation adenovirus vectors for immunization, vaccine antigens are presented to the immune system simultaneously with highly immunogenic Ad capsid proteins.

The term “Second Generation Adenovirus”, as used herein, refers to an Ad that has all or parts of the E1, E2, E3, and, in certain embodiments, E4 DNA gene sequences deleted (removed) from the virus. Compared to First Generation adenovirus vectors, certain embodiments of the Second Generation E2b deleted adenovirus vectors contain additional deletions in the DNA polymerase gene (pol) and deletions of the pre-terminal protein (pTP). E2b deleted vectors have up to a 13 kb gene-carrying capacity as compared to the 5 to 6 kb capacity of First-Generation adenovirus vectors, easily providing space for nucleic acid sequences encoding any of a variety of target antigens, including for example, the Gag, Pol and Nef genes of HIV (Amalfitano, et al. Curr Gene Ther 2/111-133 (2002)). The E2b deleted adenovirus vectors also have reduced adverse reactions as compared to First Generation adenovirus vectors (Morral, et al Hum Gene Ther 9/2709-2716 (1998); Hodges, et al. J Gene Med 2/250-259 (2000); DelloRusso, et al. Proc Natl Acad Sci USA 99/12979-12984 (2002); Reddy, et al. Mol Ther 5/63-73 (2002); (Amalfitano and Parks, et al. Curr Gene Ther 2/111-133 (2002); Amalfitano Curr Opin Mol Ther 5/362-366 (2003); Everett, et al. Human Gene Ther 14/1715-1726 (2003)) E2b deleted vectors have reduced expression of viral genes (Hodges, et al. J Gene Med 2/250-259 (2000); Amalfitano, et al. J Virol 72/926-933 (1998); Hartigan-O'Connor, et al. Mol Ther 4/525-533 (2001)), and this characteristic has been reported to lead to extended transgene expression in vivo (Hu, et al. Hum Gene Ther 10/355-364 (1999); DelloRusso, et al. Proc Natl Acad Sci USA 99/12979-12984 (2002); Reddy, et al. Mol Ther 5/63-73 (2002); (Amalfitano and Parks, et al. Curr Gene Ther 2/111-133 (2002); Amalfitano Curr Opin Mol Ther 5/362-366 (2003); Everett, et al. Human Gene Ther 14/1715-1726 (2003)).

The innate immune response to wild type Ad can be complex, and it appears that Ad proteins expressed from adenovirus vectors play an important role (Moorhead, et al. J Virol 73/1046-1053 (1999); Nazir, et al. J Investig Med 53/292-304 (2005); Schaack, et al. Proc Natl Acad Sci USA 101/3124-3129 (2004); Schaack, et al. Viral Immunol 18/79-88 (2005); Kiang, et al. Mol Ther 14/588-598 (2006); Hartman, et al. J Virol 81/1796-1812 (2007); Hartman, et al. Virology 358/357-372 (2007)). Specifically, the deletions of pre-terminal protein and DNA polymerase in the E2b deleted vectors appear to reduce inflammation during the first 24 to 72 hours following injection, whereas First Generation adenovirus vectors stimulate inflammation during this period (Schaack, et al. Proc Natl Acad Sci USA 101/3124-3129 (2004); Schaack, et al. Viral Immunol 18/79-88 (2005); Kiang, et al. Mol Ther 14/588-598 (2006); Hartman, et al. J Virol 81/1796-1812 (2007); Hartman, et al. Virology 358/357-372 (2007)). In addition, it has been reported that the additional replication block created by E2b deletion also leads to a 10,000 fold reduction in expression of Ad late genes, well beyond that afforded by E1, E3 deletions alone (Amalfitano et al. J. Virol. 72/926-933 (1998); Hodges et al. J. Gene Med. 2/250-259 (2000)). The decreased levels of Ad proteins produced by E2b deleted adenovirus vectors effectively reduce the potential for competitive, undesired, immune responses to Ad antigens, responses that prevent repeated use of the platform in Ad immunized or exposed individuals. The reduced induction of inflammatory response by Second Generation E2b deleted vectors results in increased potential for the vectors to express desired vaccine antigens during the infection of antigen presenting cells (i.e. dendritic cells), decreasing the potential for antigenic competition, resulting in greater immunization of the vaccine to the desired antigen relative to identical attempts with First Generation adenovirus vectors. E2b deleted adenovirus vectors provide an improved Ad-based vaccine candidate that is safer, more effective, and more versatile than previously described vaccine candidates using First Generation adenovirus vectors.

Thus, the present invention contemplates the use of Second-Generation adenovirus vectors, preferably E2b deleted adenovirus vectors, such as those described in U.S. Pat. Nos. 6,063,622; 6,451,596; 6,057,158: and 6,083,750. As described in the '622 patent, in order to further cripple viral protein expression, and also to decrease the frequency of generating replication competent Ad (RCA), the present invention provides adenovirus vectors containing deletions in the E2b region. Propagation of these E2b deleted adenovirus vectors requires cell lines that express the deleted E2b gene products. The present invention also provides such packaging cell lines; for example E.C7 (formally called C-7), derived from the HEK-203 cell line (Amalfitano, et al. Proc Natl Acad Sci USA 93/3352-3356 (1996); Amalfitano, et al. Gene Ther 4/258-263 (1997)).

Further, the E2b gene products, DNA polymerase and preterminal protein, can be constitutively expressed in E.C7, or similar cells along with the E1 gene products. Transfer of gene segments from the Ad genome to the production cell line has immediate benefits: (1) increased carrying capacity of the recombinant DNA polymerase and preterminal protein-deleted adenovirus vector, since the combined coding sequences of the DNA polymerase and preterminal proteins that can be theoretically deleted approaches 4.6 kb; and, (2) a decreased potential of RCA generation, since two or more independent recombination events would be required to generate RCA. Therefore, the E1, Ad DNA polymerase and preterminal protein expressing cell lines used in the present invention enable the propagation of adenovirus vectors with a carrying capacity approaching 13 kb, without the need for a contaminating helper virus [Mitani et al. (1995) Proc. Natl. Acad. Sci. USA 92:3854; Hodges, et al., 2000 J Gene Med 2:250-259; (Amalfitano and Parks, Curr Gene Ther 2/111-133 (2002)]. In addition, when genes critical to the viral life cycle are deleted (e.g., the E2b genes), a further crippling of Ad to replicate or express other viral gene proteins occurs. This will decrease immune recognition of virally infected cells, and allows for extended durations of foreign transgene expression.

The most important attribute of E1, DNA polymerase, and preterminal protein deleted vectors, however, is their inability to express the respective proteins from the E1 and E2b regions, as well as a predicted lack of expression of most of the viral structural proteins. For example, the major late promoter (MLP) of Ad is responsible for transcription of the late structural proteins L1 through L5 [Doerfler, In Adenovirus DNA, The Viral Genome and Its Expression (Martinus Nijhoff Publishing Boston, 1986)]. Though the MLP is minimally active prior to Ad genome replication, the highly toxic Ad late genes are primarily transcribed and translated from the MLP only after viral genome replication has occurred [Thomas and Mathews (1980) Cell 22:523]. This cis-dependent activation of late gene transcription is a feature of DNA viruses in general, such as in the growth of polyoma and SV-40. The DNA polymerase and preterminal proteins are absolutely required for Ad replication (unlike the E4 or protein IX proteins) and thus their deletion is extremely detrimental to adenovirus vector late gene expression, and the toxic effects of that expression in cells such as APCs.

In certain embodiments, the adenovirus vectors contemplated for use in the present invention include E2b deleted adenovirus vectors that have a deletion in the E2b region of the Ad genome and the E1 region but do not have any other regions of the Ad genome deleted. In another embodiment, the adenovirus vectors contemplated for use in the present invention include E2b deleted adenovirus vectors that have a deletion in the E2b region of the Ad genome and deletions in the E1 and E3 regions, but no other regions deleted. In a further embodiment, the adenovirus vectors contemplated for use in the present invention include adenovirus vectors that have a deletion in the E2b region of the Ad genome and deletions in the E1, E3 and partial or complete removal of the E4 regions but no other deletions. In another embodiment, the adenovirus vectors contemplated for use in the present invention include adenovirus vectors that have a deletion in the E2b region of the Ad genome and deletions in the E1 and E4 regions but no other deletions. In an additional embodiment, the adenovirus vectors contemplated for use in the present invention include adenovirus vectors that have a deletion in the E2a, E2b and E4 regions of the Ad genome but no other deletions. In one embodiment, the adenovirus vectors for use herein comprise vectors having the E1 and DNA polymerase functions of the E2b region deleted but no other deletions. In a further embodiment, the adenovirus vectors for use herein have the E1 and the preterminal protein functions of the E2b region deleted and no other deletions. In another embodiment, the adenovirus vectors for use herein have the E1, DNA polymerase and the preterminal protein functions deleted, and no other deletions. In one particular embodiment, the adenovirus vectors contemplated for use herein are deleted for at least a portion of the E2b region and the E1 region, but are not “gutted” adenovirus vectors. The term “gutted” or “gutless”, as used herein, refers to an adenovirus vector that has been deleted of all viral coding regions. In this regard, the vectors may be deleted for both the DNA polymerase and the preterminal protein functions of the E2b region. In an additional embodiment, the adenovirus vectors for use in the present invention include adenovirus vectors that have a deletion in the E1, E2b and 100K regions of the adenovirus genome. In one embodiment, the adenovirus vectors for use herein comprise vectors having the E1, E2b and protease functions deleted but no other deletions. In a further embodiment, the adenovirus vectors for use herein have the E1 and the E2b regions deleted, while the fiber genes have been modified by mutation or other alterations (for example to alter Ad tropism). Removal of genes from the E3 or E4 regions may be added to any of the mentioned adenovirus vectors. In certain embodiments, the adenovirus vector may be a “gutted” adenovirus vector.

The term “E2b deleted”, as used herein, refers to a specific DNA sequence that is mutated in such a way so as to prevent expression and/or function of at least one E2b gene product. Thus, in certain embodiments, “E2b deleted” refers to a specific DNA sequence that is deleted (removed) from the Ad genome. E2b deleted or “containing a deletion within the E2b region” refers to a deletion of at least one base pair within the E2b region of the Ad genome. Thus, in certain embodiments, more than one base pair is deleted and in further embodiments, at least 20, 30, 40, 50, 60, 70, 80, 90, 100, 110, 120, 130, 140, or 150 base pairs are deleted. In another embodiment, the deletion is of more than 150, 160, 170, 180, 190, 200, 250, or 300 base pairs within the E2b region of the Ad genome. An E2b deletion may be a deletion that prevents expression and/or function of at least one E2b gene product and therefore, encompasses deletions within exons encoding portions of E2b-specific proteins as well as deletions within promoter and leader sequences. In certain embodiments, an E2b deletion is a deletion that prevents expression and/or function of one or both of the DNA polymerase and the preterminal protein of the E2b region. In a further embodiment, “E2b deleted” refers to one or more point mutations in the DNA sequence of this region of an Ad genome such that one or more encoded proteins is non-functional. Such mutations include residues that are replaced with a different residue leading to a change in the amino acid sequence that result in a nonfunctional protein.

As would be understood by the skilled artisan upon reading the present disclosure, other regions of the Ad genome can be deleted. Thus, to be “deleted” in a particular region of the Ad genome, as used herein, refers to a specific DNA sequence that is mutated in such a way so as to prevent expression and/or function of at least one gene product encoded by that region. In certain embodiments, to be “deleted” in a particular region refers to a specific DNA sequence that is deleted (removed) from the Ad genome in such a way so as to prevent the expression and/or the function encoded by that region (e.g., E2b functions of DNA polymerase or preterminal protein function). “Deleted” or “containing a deletion” within a particular region refers to a deletion of at least one base pair within that region of the Ad genome. Thus, in certain embodiments, more than one base pair is deleted and in further embodiments, at least 20, 30, 40, 50, 60, 70, 80, 90, 100, 110, 120, 130, 140, or 150 base pairs are deleted from a particular region. In another embodiment, the deletion is more than 150, 160, 170, 180, 190, 200, 250, or 300 base pairs within a particular region of the Ad genome. These deletions are such that expression and/or function of the gene product encoded by the region is prevented. Thus, deletions encompass deletions within exons encoding portions of proteins as well as deletions within promoter and leader sequences. In a further embodiment, “deleted” in a particular region of the Ad genome refers to one or more point mutations in the DNA sequence of this region of an Ad genome such that one or more encoded proteins is non-functional. Such mutations include residues that are replaced with a different residue leading to a change in the amino acid sequence that result in a nonfunctional protein.

The deleted adenovirus vectors of the present invention can be generated using recombinant techniques known in the art (see e.g., Amalfitano et al., 1998 J. Virol. 72:926-933; Hodges, et al., 2000 J Gene Med 2:250-259).

As would be recognized by the skilled artisan, the adenovirus vectors for use in the present invention can be successfully grown to high titers using an appropriate packaging cell line that constitutively expresses E2b gene products and products of any of the necessary genes that may have been deleted. In certain embodiments, HEK-293-derived cells that not only constitutively express the E1 and DNA polymerase proteins, but also the Ad-preterminal protein, can be used. In one embodiment, E.C7 cells are used to successfully grow high titer stocks of the adenovirus vectors (see e.g., Amalfitano et al., J. Virol. 1998 72:926-933; Hodges, et al. J Gene Med 2/250-259 (2000))

In order to delete critical genes from self-propagating adenovirus vectors, the proteins encoded by the targeted genes have to first be coexpressed in HEK-293 cells, or similar, along with the E1 proteins. Therefore, only those proteins which are non-toxic when coexpressed constitutively (or toxic proteins inducibly-expressed) can be utilized. Coexpression in HEK-293 cells of the E1 and E4 genes has been demonstrated (utilizing inducible, not constitutive, promoters) [Yeh et al. (1996) J. Virol. 70:559; Wang et al. (1995) Gene Therapy 2:775; and Gorziglia et al. (1996) J. Virol. 70:4173]. The E1 and protein IX genes (a virion structural protein) have been coexpressed [Caravokyri and Leppard (1995) J. Virol. 69:6627], and coexpression of the E1, E4, and protein IX genes has also been described [Krougliak and Graham (1995) Hum. Gene Ther. 6:1575]. The E1 and 100 k genes have been successfully expressed in transcomplementing cell lines, as have E1 and protease genes (Oualikene, et al. Hum Gene Ther 11/1341-1353 (2000); Hodges, et al. J. Virol 75/5913-5920 (2001)).

Cell lines coexpressing E1 and E2b gene products for use in growing high titers of E2b deleted Ad particles are described in U.S. Pat. No. 6,063,622. The E2b region encodes the viral replication proteins which are absolutely required for Ad genome replication [Doerfler, supra and Pronk et al. (1992) Chromosoma 102:S39-S45]. Useful cell lines constitutively express the approximately 140 kD Ad-DNA polymerase and/or the approximately 90 kD preterminal protein. In particular, cell lines that have high-level, constitutive coexpression of the E1, DNA polymerase, and preterminal proteins, without toxicity (e.g. E.C7), are desirable for use in propagating Ad for use in multiple vaccinations. These cell lines permit the propagation of adenovirus vectors deleted for the E1, DNA polymerase, and preterminal proteins.

The recombinant Ad of the present invention can be propagated using techniques known in the art. For example, in certain embodiments, tissue culture plates containing E.C7 cells are infected with the adenovirus vector virus stocks at an appropriate MOI (e.g., 5) and incubated at 37.0° C. for 40-96 h. The infected cells are harvested, resuspended in 10 mM Tris-Cl (pH 8.0), and sonicated, and the virus is purified by two rounds of cesium chloride density centrifugation. In certain techniques, the virus containing band is desalted over a Sephadex CL-6B column (Pharmacia Biotech, Piscataway, N.J.), sucrose or glycerol is added, and aliquots are stored at −80° C. In some embodiments, the virus will be placed in a solution designed to enhance its stability, such as A195 (Evans, et al. J Pharm Sci 93/2458-2475 (2004)) The titer of the stock is measured (e.g., by measurement of the optical density at 260 nm of an aliquot of the virus after SDS lysis). In another embodiment, plasmid DNA, either linear or circular, encompassing the entire recombinant E2b deleted adenovirus vector can be transfected into E.C7, or similar cells, and incubated at 37.0° C. until evidence of viral production is present (e.g. the cytopathic effect). The conditioned media from these cells can then be used to infect more E.C7, or similar cells, to expand the amount of virus produced, before purification. Purification can be accomplished by two rounds of cesium chloride density centrifugation or selective filtration. In certain embodiments, the virus may be purified by column chromatography, using commercially available products (e.g. Adenopure from Puresyn, Inc., Malvern, Pa.) or custom made chromatographic columns.

Generally, the recombinant Ad of the present invention comprises enough of the virus to ensure that the cells to be infected are confronted with a certain number of viruses. Thus, the present invention provides a stock of recombinant Ad, preferably an RCA-free stock of recombinant Ad. The preparation and analysis of Ad stocks is well known in the art. Viral stocks vary considerably in titer, depending largely on viral genotype and the protocol and cell lines used to prepare them. The viral stocks of the present invention can have a titer of at least about 106, 107, or 108 pfu/ml, and many such stocks can have higher titers, such as at least about 109, 1010, 1011, or 1012 pfu/ml. Depending on the nature of the recombinant virus and the packaging cell line, it is possible that a viral stock of the present invention can have a titer of even about 1013 particles/ml or higher.

Further information on viral delivery systems is known in the art and can be found, for example, in Fisher-Hoch et al., Proc. Natl. Acad. Sci. USA 86:317-321, 1989; Flexner et al., Ann. N.Y. Acad. Sci. 569:86-103, 1989; Flexner et al., Vaccine 8:17-21, 1990; U.S. Pat. Nos. 4,603,112, 4,769,330, and 5,017,487; WO 89/01973; U.S. Pat. No. 4,777,127; GB 2,200,651; EP 0,345,242; WO 91/02805; Berkner, Biotechniques 6:616-627, 1988; Rosenfeld et al., Science 252:431-434, 1991; Kolls et al., Proc. Natl. Acad. Sci. USA 91:215-219, 1994; Kass-Eisler et al., Proc. Natl. Acad. Sci. USA 90:11498-11502, 1993; Guzman et al., Circulation 88:2838-2848, 1993; and Guzman et al., Cir. Res. 73:1202-1207, 1993.

### Nucleic Acid-Based Vaccines

Nucleic acid-based vaccines include vaccines include one or more of deoxyribonucleic acids (DNAs), ribonucleic acids (RNAs), threose nucleic acids (TNAs), glycol nucleic acids (GNAs), peptide nucleic acids (PNAs), locked nucleic acids (LNAs, including LNA having a β-D-ribo configuration, α-LNA having an α-L-ribo configuration (a diastereomer of LNA), 2′-amino-LNA having a 2′-amino functionalization, and 2′-amino-α-LNA having a 2′-amino functionalization), ethylene nucleic acids (ENA), cyclohexenyl nucleic acids (CeNA) and/or chimeras and/or combinations thereof.

Non-viral delivered nucleic acid-based vaccines mimics infection or immunization with live microorganisms and stimulates potent T follicular helper and germinal center B cell immune response. In one embodiment, the vaccine is a DNA vaccine comprising of a synthetic DNA construct encoding the vaccine antigen. The DNA vaccine may be injected into a subject as bare nucleic acid. Enhanced delivery technologies, such as electroporation, may be used to increase the efficacy of DNA vaccines.

In a more preferred embodiment, the vaccine is an RNA vaccine. The vaccines of the present disclosure comprise at least one (one or more) ribonucleic acid (RNA) having an open reading frame encoding at least one antigen. In some embodiments, the RNA is a messenger RNA (mRNA) having an open reading frame encoding at least one antigen. In some embodiments, the RNA (e.g., mRNA) further comprises a (at least one) 5′UTR, 3′UTR, a polyA tail and/or a 5′ cap.

Messenger RNA (mRNA) is any ribonucleic acid that encodes a (at least one) protein, including a naturally occurring, non-naturally-occurring, or modified polymer of amino acids and can be translated to produce the encoded protein in vitro, in vivo, in situ, or ex vivo. The skilled artisan will appreciate that, except where otherwise noted, nucleic acid sequences set forth in the instant application may recite “T”s in a representative DNA sequence but where the sequence represents RNA (e.g., mRNA), the “T”s would be substituted for “U”s. Thus, any of the DNAs disclosed and identified by a particular sequence identification number herein also disclose the corresponding RNA (e.g., mRNA) sequence complementary to the DNA, where each “T” of the DNA sequence is substituted with “U.”

It should be understood that the mRNA polynucleotides of the vaccines as provided herein are synthetic molecules, i.e., they are not naturally occurring molecules. That is, the mRNA polynucleotides of the present disclosure are isolated mRNA polynucleotides. As is known in the art, “isolated polynucleotides” refer to polynucleotides that are substantially physically separated from other cellular material (e.g., separated from cells and/or systems that produce the polynucleotides) or from other material that hinders their use in the vaccines of the present disclosure. Isolated polynucleotides are substantially pure in that they have been substantially separated from the substances with which they may be associated in living or viral systems. Thus, mRNA polynucleotide vaccines are not associated with living or viral systems, such as cells or viruses. The mRNA polynucleotide vaccines do not include viral components (e.g., viral capsids, viral enzymes, or other viral proteins, for example, those needed for viral-based replication), and the mRNA polynucleotide vaccines are not packaged within, encapsulated within, linked to, or otherwise associated with a virus or viral particle. In some embodiments, the mRNA vaccines comprise a lipid nanoparticle that consists of, or consists essentially of, one or more mRNA polynucleotides (e.g., mRNA polynucleotides encoding one or more viral antigen(s)).

An open reading frame (ORF) is a continuous stretch of DNA or RNA beginning with a start codon (e.g., methionine (ATG or AUG)) and ending with a stop codon (e.g., TAA, TAG or TGA, or UAA, UAG or UGA). An ORF typically encodes a protein but may be engineered to encode a protein fragment, such as an immunogenic fragment of a PoMV. It will be understood that the sequences disclosed herein may further comprise additional elements, e.g., 5′ and 3′ UTRs, but that those elements, unlike the ORF, need not necessarily be present in a vaccine of the present disclosure.

In some embodiments, an RNA of the present disclosure encodes an antigen variant. Antigen or other polypeptide variants refers to molecules that differ in their amino acid sequence from a wild-type, native or reference sequence. The antigen/polypeptide variants may possess substitutions, deletions, and/or insertions at certain positions within the amino acid sequence, as compared to a native or reference sequence. Ordinarily, variants possess at least 50% identity to a wild-type, native or reference sequence. In some embodiments, variants share at least 80%, or at least 90% identity with a wild-type, native or reference sequence.

Variant antigens/polypeptides encoded by nucleic acids of the disclosure may contain amino acid changes that confer any of a number of desirable properties, e.g., that enhance their immunogenicity, enhance their expression, and/or improve their stability or PK/PD properties in a subject. Variant antigens/polypeptides can be made using routine mutagenesis techniques and assayed as appropriate to determine whether they possess the desired property. Assays to determine expression levels and immunogenicity are well known in the art. Similarly, PK/PD properties of a protein variant can be measured using art recognized techniques, e.g., by determining expression of antigens in a vaccinated subject over time and/or by looking at the durability of the induced immune response. The stability of protein(s) encoded by a variant nucleic acid may be measured by assaying thermal stability or stability upon urea denaturation or may be measured using in silico prediction. Methods for such experiments and in silico determinations are known in the art.

In some embodiments, a vaccine comprises an mRNA ORF having a nucleotide sequence identified by any one of the sequences provided herein, or having a nucleotide sequence at least 80%, at least 85%, at least 90%, at least 95%, at least 96%, at least 97%, at least 98%, or at least 99% identical to a nucleotide sequence identified by any one of the sequence provided herein.

As such, polynucleotides encoding peptides or polypeptides containing substitutions, insertions and/or additions, deletions and covalent modifications with respect to reference sequences, in particular the polypeptide (e.g., antigen) sequences disclosed herein, are included within the scope of this disclosure. For example, sequence tags or amino acids, such as one or more lysines, can be added to peptide sequences (e.g., at the N-terminal or C-terminal ends). Sequence tags can be used for peptide detection, purification or localization. Lysines can be used to increase peptide solubility or to allow for biotinylation. Alternatively, amino acid residues located at the carboxy and amino terminal regions of the amino acid sequence of a peptide or protein may optionally be deleted providing for truncated sequences. Certain amino acids (e.g., C-terminal or N-terminal residues) may alternatively be deleted depending on the use of the sequence, as for example, expression of the sequence as part of a larger sequence which is soluble, or linked to a solid support. In some embodiments, sequences for (or encoding) signal sequences, termination sequences, transmembrane domains, linkers, multimerization domains (such as, e.g., foldon regions) and the like may be substituted with alternative sequences that achieve the same or a similar function. In some embodiments, cavities in the core of proteins can be filled to improve stability, e.g., by introducing larger amino acids. In other embodiments, buried hydrogen bond networks may be replaced with hydrophobic resides to improve stability. In yet other embodiments, glycosylation sites may be removed and replaced with appropriate residues. Such sequences are readily identifiable to one of skill in the art. It should also be understood that some of the sequences provided herein contain sequence tags or terminal peptide sequences (e.g., at the N-terminal or C-terminal ends) that may be deleted, for example, prior to use in the preparation of an RNA (e.g., mRNA) vaccine.

As recognized by those skilled in the art, protein fragments, functional protein domains, and homologous proteins are also considered to be within the scope of antigens of interest. For example, provided herein is any protein fragment of a reference protein, provided that the fragment is immunogenic and confers a protective immune response to the pathogen. In addition to variants that are identical to the reference protein but are truncated, in some embodiments, an antigen includes 2, 3, 4, 5, 6, 7, 8, 9, 10, or more mutations, as shown in any of the sequences provided or referenced herein. Antigens/antigenic polypeptides can range in length from about 4, 6, or 8 amino acids to full length proteins.

Naturally-occurring eukaryotic mRNA molecules can contain stabilizing elements, including, but not limited to untranslated regions (UTR) at their 5′-end (5′ UTR) and/or at their 3′-end (3′ UTR), in addition to other structural features, such as a 5′-cap structure or a 3′-poly(A) tail. Both the 5′ UTR and the 3′ UTR are typically transcribed from the genomic DNA and are elements of the premature mRNA. Characteristic structural features of mature mRNA, such as the 5′-cap and the 3′-poly(A) tail are usually added to the transcribed (premature) mRNA during mRNA processing.

In some embodiments, a vaccine includes at least one RNA polynucleotide having an open reading frame encoding at least one antigenic polypeptide having at least one modification, at least one 5′ terminal cap, and is formulated within a lipid nanoparticle. 5′-capping of polynucleotides may be completed concomitantly during the in vitro-transcription reaction using the following chemical RNA cap analogs to generate the 5′-guanosine cap structure according to manufacturer protocols: 3′-O-Me-m7G(5′)ppp(5′) G [the ARCA cap]; G(5′)ppp(5′)A; G(5′)ppp(5′)G; m7G(5′)ppp(5′)A; m7G(5′)ppp(5′)G (New England BioLabs, Ipswich, Mass.). 5′-capping of modified RNA may be completed post-transcriptionally using a Vaccinia Virus Capping Enzyme to generate the “Cap 0” structure: m7G(5′)ppp(5′)G (New England BioLabs, Ipswich, Mass.). Cap 1 structure may be generated using both Vaccinia Virus Capping Enzyme and a 2′-O methyl-transferase to generate: m7G(5′)ppp(5′)G-2′-O-methyl. Cap 2 structure may be generated from the Cap 1 structure followed by the 2′-O-methylation of the 5′-antepenultimate nucleotide using a 2′-O methyl-transferase. Cap 3 structure may be generated from the Cap 2 structure followed by the 2′-O-methylation of the 5′-preantepenultimate nucleotide using a 2′-O methyl-transferase. Enzymes may be derived from a recombinant source.

The 3′-poly(A) tail is typically a stretch of adenine nucleotides added to the 3′-end of the transcribed mRNA. It can, in some instances, comprise up to about 400 adenine nucleotides. In some embodiments, the length of the 3′-poly(A) tail may be an essential element with respect to the stability of the individual mRNA.

In some embodiments, RNA vaccines may include one or more stabilizing elements. Stabilizing elements may include for instance a histone stem-loop. A stem-loop binding protein (SLBP), a 32 kDa protein has been identified. It is associated with the histone stem-loop at the 3′-end of the histone messages in both the nucleus and the cytoplasm. Its expression level is regulated by the cell cycle; it peaks during the S-phase, when histone mRNA levels are also elevated. The protein has been shown to be essential for efficient 3′-end processing of histone pre-mRNA by the U7 snRNP. SLBP continues to be associated with the stem-loop after processing, and then stimulates the translation of mature histone mRNAs into histone proteins in the cytoplasm. The RNA binding domain of SLBP is conserved through metazoa and protozoa; its binding to the histone stem-loop depends on the structure of the loop. The minimum binding site includes at least three nucleotides 5′ and two nucleotides 3′ relative to the stem-loop.

In some embodiments, RNA vaccines include a coding region, at least one histone stem-loop, and optionally, a poly(A) sequence or polyadenylation signal. The poly(A) sequence or polyadenylation signal generally should enhance the expression level of the encoded protein. The encoded protein, in some embodiments, is not a histone protein, a reporter protein (e.g. Luciferase, GFP, EGFP, β-Galactosidase, EGFP), or a marker or selection protein (e.g. alpha-Globin, Galactokinase and Xanthine:guanine phosphoribosyl transferase (GPT)).

In some embodiments, the combination of a poly(A) sequence or polyadenylation signal and at least one histone stem-loop, even though both represent alternative mechanisms in nature, acts synergistically to increase the protein expression beyond the level observed with either of the individual elements. The synergistic effect of the combination of poly(A) and at least one histone stem-loop does not depend on the order of the elements or the length of the poly(A) sequence.

In some embodiments, RNA vaccines do not comprise a histone downstream element (HDE). “Histone downstream element” (HDE) includes a purine-rich polynucleotide stretch of approximately 15 to 20 nucleotides 3′ of naturally occurring stem-loops, representing the binding site for the U7 snRNA, which is involved in processing of histone pre-mRNA into mature histone mRNA. In some embodiments, the nucleic acid does not include an intron.

In some embodiments, RNA vaccines may or may not contain an enhancer and/or promoter sequence, which may be modified or unmodified or which may be activated or inactivated. In some embodiments, the histone stem-loop is generally derived from histone genes, and includes an intramolecular base pairing of two neighbored partially or entirely reverse complementary sequences separated by a spacer, consisting of a short sequence, which forms the loop of the structure. The unpaired loop region is typically unable to base pair with either of the stem loop elements. It occurs more often in RNA, as is a key component of many RNA secondary structures, but may be present in single-stranded DNA as well. Stability of the stem-loop structure generally depends on the length, number of mismatches or bulges, and base composition of the paired region. In some embodiments, wobble base pairing (non-Watson-Crick base pairing) may result. In some embodiments, the at least one histone stem-loop sequence comprises a length of 15 to 45 nucleotides.

In some embodiments, RNA vaccines may have one or more AU-rich sequences removed. These sequences, sometimes referred to as AURES are destabilizing sequences found in the 3′UTR. The AURES may be removed from the RNA vaccines. Alternatively the AURES may remain in the RNA vaccine.

In some embodiments, a vaccine comprises a RNA having an ORF that encodes a signal peptide fused to the antigen. Signal peptides, comprising the N-terminal 15-60 amino acids of proteins, are typically needed for the translocation across the membrane on the secretory pathway and, thus, universally control the entry of most proteins both in eukaryotes and prokaryotes to the secretory pathway. In eukaryotes, the signal peptide of a nascent precursor protein (pre-protein) directs the ribosome to the rough endoplasmic reticulum (ER) membrane and initiates the transport of the growing peptide chain across it for processing. ER processing produces mature proteins, wherein the signal peptide is cleaved from precursor proteins, typically by a ER-resident signal peptidase of the host cell, or they remain uncleaved and function as a membrane anchor. A signal peptide may also facilitate the targeting of the protein to the cell membrane.

A signal peptide may have a length of 15-60 amino acids. For example, a signal peptide may have a length of 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 amino acids. In some embodiments, a signal peptide has a length of 20-60, 25-60, 30-60, 35-60, 40-60, 45-60, 50-60, 55-60, 15-55, 20-55, 25-55, 30-55, 35-55, 40-55, 45-55, 50-55, 15-50, 20-50, 25-50, 30-50, 35-50, 40-50, 45-50, 15-45, 20-45, 25-45, 30-45, 35-45, 40-45, 15-40, 20-40, 25-40, 30-40, 35-40, 15-35, 20-35, 25-35, 30-35, 15-30, 20-30, 25-30, 15-25, 20-25, or 15-20 amino acids.

Signal peptides from heterologous genes (which regulate expression of genes other than antigens in nature) are known in the art and can be tested for desired properties and then incorporated into a nucleic acid of the disclosure. In some embodiments, the signal peptide may comprise one of the following sequences:

MDSKGSSQKGSRLLLLLVVSNLLLPQGVVG,
MDWTWILFLVAAATRVHS;
METPAQLLFLLLLWLPDTTG;
MLGSNSGQRVVFTILLLLVAPAYS;
MKCLLYLAFLFIGVNCA;
MWLVSLAIVTACAGA.

In some embodiments, an RNA vaccine of the present disclosure includes an RNA encoding an antigenic fusion protein. Thus, the encoded antigen or antigens may include two or more proteins (e.g., protein and/or protein fragment) joined together.

Alternatively, the protein to which a protein antigen is fused does not promote a strong immune response to itself, but rather to the antigen. Antigenic fusion proteins, in some embodiments, retain the functional property from each original protein.

The RNA (e.g., mRNA) vaccines as provided herein, in some embodiments, encode fusion proteins which comprise antigens linked to scaffold moieties. In some embodiments, such scaffold moieties impart desired properties to an antigen encoded by a nucleic acid of the disclosure. For example, scaffold proteins may improve the immunogenicity of an antigen, e.g., by altering the structure of the antigen, altering the uptake and processing of the antigen, and/or causing the antigen to bind to a binding partner.

In some embodiments, the scaffold moiety is protein that can self-assemble into protein nanoparticles that are highly symmetric, stable, and structurally organized, with diameters of 10-150 nm, a highly suitable size range for optimal interactions with various cells of the immune system. In some embodiments, viral proteins or virus-like particles can be used to form stable nanoparticle structures. Examples of such viral proteins are known in the art. For example, in some embodiments, the scaffold moiety is a hepatitis B surface antigen (HBsAg). HBsAg forms spherical particles with an average diameter of −22 nm and which lacked nucleic acid and hence are non-infectious (Lopez-Sagaseta, J. et al. Computational and Structural Biotechnology Journal 14 (2016) 58-68). In some embodiments, the scaffold moiety is a hepatitis B core antigen (HBcAg) self-assembles into particles of 24-31 nm diameter, which resembled the viral cores obtained from HBV-infected human liver. HBcAg produced in self-assembles into two classes of differently sized nanoparticles of 300 Å and 360 Å diameter, corresponding to 180 or 240 protomers. In some embodiments a antigen is fused to HBsAG or HBcAG to facilitate self-assembly of nanoparticles displaying the antigen.

In another embodiment, bacterial protein platforms may be used. Non-limiting examples of these self-assembling proteins include ferritin, lumazine and encapsulin.

Ferritin is a protein whose main function is intracellular iron storage. Ferritin is made of 24 subunits, each composed of a four-alpha-helix bundle, that self-assemble in a quaternary structure with octahedral symmetry (Cho K. J. et al. J Mol Biol. 2009; 390:83-98). Several high-resolution structures of ferritin have been determined, confirming that Helicobacter pylori ferritin is made of 24 identical protomers, whereas in animals, there are ferritin light and heavy chains that can assemble alone or combine with different ratios into particles of 24 subunits (Granier T. et al. J Biol Inorg Chem. 2003; 8:105-111; Lawson D. M. et al. Nature. 1991; 349:541-544). Ferritin self-assembles into nanoparticles with robust thermal and chemical stability. Thus, the ferritin nanoparticle is well-suited to carry and expose antigens.

Lumazine synthase (LS) is also well-suited as a nanoparticle platform for antigen display. LS, which is responsible for the penultimate catalytic step in the biosynthesis of riboflavin, is an enzyme present in a broad variety of organisms, including archaea, bacteria, fungi, plants, and eubacteria (Weber S. E. Flavins and Flavoproteins. Methods and Protocols, Series: Methods in Molecular Biology. 2014). The LS monomer is 150 amino acids long and consists of beta-sheets along with tandem alpha-helices flanking its sides. A number of different quaternary structures have been reported for LS, illustrating its morphological versatility: from homopentamers up to symmetrical assemblies of 12 pentamers forming capsids of 150 Å diameter. Even LS cages of more than 100 subunits have been described (Zhang X. et al. J Mol Biol. 2006; 362:753-770).

Encapsulin, a protein cage nanoparticle isolated from thermophile Thermotoga maritima, may also be used as a platform to present antigens on the surface of self-assembling nanoparticles. Encapsulin is assembled from 60 copies of identical 31 kDa monomers having a thin and icosahedral T=1 symmetric cage structure with interior and exterior diameters of 20 and 24 nm, respectively (Sutter M. et al. Nat Struct Mol Biol. 2008, 15: 939-947). Although the exact function of encapsulin in T. maritima is not clearly understood yet, its crystal structure has been recently solved and its function was postulated as a cellular compartment that encapsulates proteins such as DyP (Dye decolorizing peroxidase) and Flp (Ferritin like protein), which are involved in oxidative stress responses (Rahmanpour R. et al. FEBS J 2013, 280: 2097-2104).

In some embodiments, the mRNAs of the disclosure encode more than one polypeptide, referred to herein as fusion proteins. In some embodiments, the mRNA further encodes a linker located between at least one or each domain of the fusion protein. The linker can be, for example, a cleavable linker or protease-sensitive linker. In some embodiments, the linker is selected from the group consisting of F2A linker, P2A linker, T2A linker, E2A linker, and combinations thereof. This family of self-cleaving peptide linkers, referred to as 2A peptides, has been described in the art (see for example, Kim, J. H. et al. (2011) PLoS ONE 6:e18556). In some embodiments, the linker is an F2A linker. In some embodiments, the linker is a GGGS linker. In some embodiments, the fusion protein contains three domains with intervening linkers, having the structure: domain-linker-domain-linker-domain.

Cleavable linkers known in the art may be used in connection with the disclosure. Exemplary such linkers include: F2A linkers, T2A linkers, P2A linkers, E2A linkers (See, e.g., WO2017/127750). The skilled artisan will appreciate that other art-recognized linkers may be suitable for use in the constructs of the disclosure (e.g., encoded by the nucleic acids of the disclosure). The skilled artisan will likewise appreciate that other polycistronic constructs (mRNA encoding more than one antigen/polypeptide separately within the same molecule) may be suitable for use as provided herein.

In some embodiments, an ORF encoding an antigen of the disclosure is codon optimized. Codon optimization methods are known in the art. For example, an ORF of any one or more of the sequences provided herein may be codon optimized. Codon optimization, in some embodiments, may be used to match codon frequencies in target and host organisms to ensure proper folding; bias GC content to increase mRNA stability or reduce secondary structures; minimize tandem repeat codons or base runs that may impair gene construction or expression; customize transcriptional and translational control regions; insert or remove protein trafficking sequences; remove/add post translation modification sites in encoded protein (e.g., glycosylation sites); add, remove or shuffle protein domains; insert or delete restriction sites; modify ribosome binding sites and mRNA degradation sites; adjust translational rates to allow the various domains of the protein to fold properly; or reduce or eliminate problem secondary structures within the polynucleotide. Codon optimization tools, algorithms and services are known in the art—non-limiting examples include services from GeneArt (Life Technologies), DNA2.0 (Menlo Park Calif.) and/or proprietary methods. In some embodiments, the open reading frame (ORF) sequence is optimized using optimization algorithms.

In some embodiments, a codon optimized sequence shares less than 95% sequence identity to a naturally-occurring or wild-type sequence ORF (e.g., a naturally-occurring or wild-type mRNA sequence encoding an antigen). In some embodiments, a codon optimized sequence shares less than 90% sequence identity to a naturally-occurring or wild-type sequence (e.g., a naturally-occurring or wild-type mRNA sequence encoding an antigen). In some embodiments, a codon optimized sequence shares less than 85% sequence identity to a naturally-occurring or wild-type sequence (e.g., a naturally-occurring or wild-type mRNA sequence encoding an antigen). In some embodiments, a codon optimized sequence shares less than 80% sequence identity to a naturally-occurring or wild-type sequence (e.g., a naturally-occurring or wild-type mRNA sequence encoding an antigen). In some embodiments, a codon optimized sequence shares less than 75% sequence identity to a naturally-occurring or wild-type sequence (e.g., a naturally-occurring or wild-type mRNA sequence encoding an antigen).

In some embodiments, a codon optimized sequence shares between 65% and 85% (e.g., between about 67% and about 85% or between about 67% and about 80%) sequence identity to a naturally-occurring or wild-type sequence (e.g., a naturally-occurring or wild-type mRNA sequence encoding an antigen). In some embodiments, a codon optimized sequence shares between 65% and 75% or about 80% sequence identity to a naturally-occurring or wild-type sequence (e.g., a naturally-occurring or wild-type mRNA sequence encoding an antigen).

In some embodiments, a codon-optimized sequence encodes an antigen that is as immunogenic as, or more immunogenic than (e.g., at least 10%, at least 20%, at least 30%, at least 40%, at least 50%, at least 100%, or at least 200% more), than an antigen encoded by a non-codon-optimized sequence. In some embodiments, a codon-optimized sequence shares between 65% and 85% (e.g., between about 67% and about 85%, or between about 67% and about 80%) sequence identity to a naturally-occurring sequence or a wild-type sequence (e.g., a naturally-occurring or wild-type mRNA sequence encoding a polypeptide or protein of interest (e.g., an antigenic protein or polypeptide)). In some embodiments, a codon-optimized sequence shares between 65% and 75%, or about 80% sequence identity to a naturally-occurring sequence or wild-type sequence (e.g., a naturally-occurring or wild-type mRNA sequence encoding a polypeptide or protein of interest (e.g., an antigenic protein or polypeptide)).

When transfected into mammalian host cells, the modified mRNAs have a stability of between 12-18 hours, or greater than 18 hours, e.g., 24, 36, 48, 60, 72, or greater than 72 hours and are capable of being expressed by the mammalian host cells.

In some embodiments, a codon optimized RNA may be one in which the levels of G/C are enhanced. The G/C-content of nucleic acid molecules (e.g., mRNA) may influence the stability of the RNA. RNA having an increased amount of guanine (G) and/or cytosine (C) residues may be functionally more stable than RNA containing a large amount of adenine (A) and thymine (T) or uracil (U) nucleotides. As an example, WO02/098443 discloses a pharmaceutical composition containing an mRNA stabilized by sequence modifications in the translated region. Due to the degeneracy of the genetic code, the modifications work by substituting existing codons for those that promote greater RNA stability without changing the resulting amino acid. The approach is limited to coding regions of the RNA.

In some embodiments, at least one RNA (e.g., mRNA) of a vaccines of the present disclosure is not chemically modified and comprises the standard ribonucleotides consisting of adenosine, guanosine, cytosine and uridine. In some embodiments, nucleotides and nucleosides of the present disclosure comprise standard nucleoside residues such as those present in transcribed RNA (e.g. A, G, C, or U). In some embodiments, nucleotides and nucleosides of the present disclosure comprise standard deoxyribonucleosides such as those present in DNA (e.g. dA, dG, dC, or dT).

RNA vaccines of the present disclosure comprise, in some embodiments, at least one nucleic acid (e.g., RNA) having an open reading frame encoding at least one antigen, wherein the nucleic acid comprises nucleotides and/or nucleosides that can be standard (unmodified) or modified as is known in the art. In some embodiments, nucleotides and nucleosides of the present disclosure comprise modified nucleotides or nucleosides. Such modified nucleotides and nucleosides can be naturally-occurring modified nucleotides and nucleosides or non-naturally occurring modified nucleotides and nucleosides. Such modifications can include those at the sugar, backbone, or nucleobase portion of the nucleotide and/or nucleoside as are recognized in the art.

In some embodiments, a naturally-occurring modified nucleotide or nucleotide of the disclosure is one as is generally known or recognized in the art. Non-limiting examples of such naturally occurring modified nucleotides and nucleotides can be found, inter alia, in the widely recognized MODOMICS database.

In some embodiments, a non-naturally occurring modified nucleotide or nucleoside of the disclosure is one as is generally known or recognized in the art. Non-limiting examples of such non-naturally occurring modified nucleotides and nucleosides can be found, inter alia, in published US application Nos. PCT/US2012/058519; PCT/US2013/075177; PCT/US2014/058897; PCT/US2014/058891; PCT/US2014/070413; PCT/US2015/36773; PCT/US2015/36759; PCT/US2015/36771; or PCT/IB2017/051367 all of which are incorporated by reference herein.

Hence, nucleic acids of the disclosure (e.g., DNA nucleic acids and RNA nucleic acids, such as mRNA nucleic acids) can comprise standard nucleotides and nucleosides, naturally-occurring nucleotides and nucleosides, non-naturally-occurring nucleotides and nucleosides, or any combination thereof.

Nucleic acids of the disclosure (e.g., DNA nucleic acids and RNA nucleic acids, such as mRNA nucleic acids), in some embodiments, comprise various (more than one) different types of standard and/or modified nucleotides and nucleosides. In some embodiments, a particular region of a nucleic acid contains one, two or more (optionally different) types of standard and/or modified nucleotides and nucleosides.

In some embodiments, a modified RNA nucleic acid (e.g., a modified mRNA nucleic acid), introduced to a cell or organism, exhibits reduced degradation in the cell or organism, respectively, relative to an unmodified nucleic acid comprising standard nucleotides and nucleosides.

In some embodiments, a modified RNA nucleic acid (e.g., a modified mRNA nucleic acid), introduced into a cell or organism, may exhibit reduced immunogenicity in the cell or organism, respectively (e.g., a reduced innate response) relative to an unmodified nucleic acid comprising standard nucleotides and nucleosides.

Nucleic acids (e.g., RNA nucleic acids, such as mRNA nucleic acids), in some embodiments, comprise non-natural modified nucleotides that are introduced during synthesis or post-synthesis of the nucleic acids to achieve desired functions or properties. The modifications may be present on internucleotide linkages, purine or pyrimidine bases, or sugars. The modification may be introduced with chemical synthesis or with a polymerase enzyme at the terminal of a chain or anywhere else in the chain. Any of the regions of a nucleic acid may be chemically modified.

The present disclosure provides for modified nucleosides and nucleotides of a nucleic acid (e.g., RNA nucleic acids, such as mRNA nucleic acids). A “nucleoside” refers to a compound containing a sugar molecule (e.g., a pentose or ribose) or a derivative thereof in combination with an organic base (e.g., a purine or pyrimidine) or a derivative thereof (also referred to herein as “nucleobase”). A “nucleotide” refers to a nucleoside, including a phosphate group. Modified nucleotides may by synthesized by any useful method, such as, for example, chemically, enzymatically, or recombinantly, to include one or more modified or non-natural nucleosides. Nucleic acids can comprise a region or regions of linked nucleosides. Such regions may have variable backbone linkages. The linkages can be standard phosphodiester linkages, in which case the nucleic acids would comprise regions of nucleotides.

Modified nucleotide base pairing encompasses not only the standard adenosine-thymine, adenosine-uracil, or guanosine-cytosine base pairs, but also base pairs formed between nucleotides and/or modified nucleotides comprising non-standard or modified bases, wherein the arrangement of hydrogen bond donors and hydrogen bond acceptors permits hydrogen bonding between a non-standard base and a standard base or between two complementary non-standard base structures, such as, for example, in those nucleic acids having at least one chemical modification. One example of such non-standard base pairing is the base pairing between the modified nucleotide inosine and adenine, cytosine or uracil. Any combination of base/sugar or linker may be incorporated into nucleic acids of the present disclosure.

In some embodiments, modified nucleobases in nucleic acids (e.g., RNA nucleic acids, such as mRNA nucleic acids) comprise 1-methyl-pseudouridine (m1ψ), 1-ethyl-pseudouridine (e1ψ), 5-methoxy-uridine (mo5U), 5-methyl-cytidine (m5C), and/or pseudouridine (v). In some embodiments, modified nucleobases in nucleic acids (e.g., RNA nucleic acids, such as mRNA nucleic acids) comprise 5-methoxymethyl uridine, 5-methylthio uridine, 1-methoxymethyl pseudouridine, 5-methyl cytidine, and/or 5-methoxy cytidine. In some embodiments, the polyribonucleotide includes a combination of at least two (e.g., 2, 3, 4 or more) of any of the aforementioned modified nucleobases, including but not limited to chemical modifications.

In some embodiments, a RNA nucleic acid of the disclosure comprises 1-methyl-pseudouridine (m1ψ) substitutions at one or more or all uridine positions of the nucleic acid.

In some embodiments, a RNA nucleic acid of the disclosure comprises 1-methyl-pseudouridine (m1ψ) substitutions at one or more or all uridine positions of the nucleic acid and 5-methyl cytidine substitutions at one or more or all cytidine positions of the nucleic acid.

In some embodiments, a RNA nucleic acid of the disclosure comprises pseudouridine (ψ) substitutions at one or more or all uridine positions of the nucleic acid.

In some embodiments, a RNA nucleic acid of the disclosure comprises pseudouridine (ψ) substitutions at one or more or all uridine positions of the nucleic acid and 5-methyl cytidine substitutions at one or more or all cytidine positions of the nucleic acid.

In some embodiments, a RNA nucleic acid of the disclosure comprises uridine at one or more or all uridine positions of the nucleic acid.

In some embodiments, nucleic acids (e.g., RNA nucleic acids, such as mRNA nucleic acids) are uniformly modified (e.g., fully modified, modified throughout the entire sequence) for a particular modification. For example, a nucleic acid can be uniformly modified with 1-methyl-pseudouridine, meaning that all uridine residues in the mRNA sequence are replaced with 1-methyl-pseudouridine. Similarly, a nucleic acid can be uniformly modified for any type of nucleoside residue present in the sequence by replacement with a modified residue such as those set forth above.

The nucleic acids of the present disclosure may be partially or fully modified along the entire length of the molecule. For example, one or more or all or a given type of nucleotide (e.g., purine or pyrimidine, or any one or more or all of A, G, U, C) may be uniformly modified in a nucleic acid of the disclosure, or in a predetermined sequence region thereof (e.g., in the mRNA including or excluding the polyA tail). In some embodiments, all nucleotides X in a nucleic acid of the present disclosure (or in a sequence region thereof) are modified nucleotides, wherein X may be any one of nucleotides A, G, U, C, or any one of the combinations A+G, A+U, A+C, G+U, G+C, U+C, A+G+U, A+G+C, G+U+C or A+G+C.

The nucleic acid may contain from about 1% to about 100% modified nucleotides (either in relation to overall nucleotide content, or in relation to one or more types of nucleotide, i.e., any one or more of A, G, U or C) or any intervening percentage (e.g., from 1% to 20%, from 1% to 25%, from 1% to 50%, from 1% to 60%, from 1% to 70%, from 1% to 80%, from 1% to 90%, from 1% to 95%, from 10% to 20%, from 10% to 25%, from 10% to 50%, from 10% to 60%, from 10% to 70%, from 10% to 80%, from 10% to 90%, from 10% to 95%, from 10% to 100%, from 20% to 25%, from 20% to 50%, from 20% to 60%, from 20% to 70%, from 20% to 80%, from 20% to 90%, from 20% to 95%, from 20% to 100%, from 50% to 60%, from 50% to 70%, from 50% to 80%, from 50% to 90%, from 50% to 95%, from 50% to 100%, from 70% to 80%, from 70% to 90%, from 70% to 95%, from 70% to 100%, from 80% to 90%, from 80% to 95%, from 80% to 100%, from 90% to 95%, from 90% to 100%, and from 95% to 100%). It will be understood that any remaining percentage is accounted for by the presence of unmodified A, G, U, or C.

The nucleic acids may contain at a minimum 1% and at maximum 100% modified nucleotides, or any intervening percentage, such as at least 5% modified nucleotides, at least 10% modified nucleotides, at least 25% modified nucleotides, at least 50% modified nucleotides, at least 80% modified nucleotides, or at least 90% modified nucleotides. For example, the nucleic acids may contain a modified pyrimidine such as a modified uracil or cytosine. In some embodiments, at least 5%, at least 10%, at least 25%, at least 50%, at least 80%, at least 90% or 100% of the uracil in the nucleic acid is replaced with a modified uracil (e.g., a 5-substituted uracil). The modified uracil can be replaced by a compound having a single unique structure, or can be replaced by a plurality of compounds having different structures (e.g., 2, 3, 4 or more unique structures). In some embodiments, at least 5%, at least 10%, at least 25%, at least 50%, at least 80%, at least 90% or 100% of the cytosine in the nucleic acid is replaced with a modified cytosine (e.g., a 5-substituted cytosine). The modified cytosine can be replaced by a compound having a single unique structure, or can be replaced by a plurality of compounds having different structures (e.g., 2, 3, 4 or more unique structures).

The nucleic acids of the present disclosure may comprise one or more regions or parts which act or function as an untranslated region. Where nucleic acids are designed to encode at least one antigen of interest, the nucleic may comprise one or more of these untranslated regions (UTRs). Wild-type untranslated regions of a nucleic acid are transcribed but not translated. In mRNA, the 5′ UTR starts at the transcription start site and continues to the start codon but does not include the start codon; whereas, the 3′ UTR starts immediately following the stop codon and continues until the transcriptional termination signal. There is growing body of evidence about the regulatory roles played by the UTRs in terms of stability of the nucleic acid molecule and translation. The regulatory features of a UTR can be incorporated into the polynucleotides of the present disclosure to, among other things, enhance the stability of the molecule. The specific features can also be incorporated to ensure controlled down-regulation of the transcript in case they are misdirected to undesired organs sites. A variety of 5′UTR and 3′UTR sequences are known and available in the art.

A 5′ UTR is region of an mRNA that is directly upstream (5′) from the start codon (the first codon of an mRNA transcript translated by a ribosome). A 5′ UTR does not encode a protein (is non-coding). Natural 5′UTRs have features that play roles in translation initiation. They harbor signatures like Kozak sequences which are commonly known to be involved in the process by which the ribosome initiates translation of many genes. Kozak sequences have the consensus CCR(A/G)CCAUGG, where R is a purine (adenine or guanine) three bases upstream of the start codon (AUG), which is followed by another ‘G’0.5′UTR also have been known to form secondary structures which are involved in elongation factor binding.

In some embodiments of the disclosure, a 5′ UTR is a heterologous UTR, i.e., is a UTR found in nature associated with a different ORF. In another embodiment, a 5′ UTR is a synthetic UTR, i.e., does not occur in nature. Synthetic UTRs include UTRs that have been mutated to improve their properties, e.g., which increase gene expression as well as those which are completely synthetic. Exemplary 5′ UTRs include Xenopus or human derived a-globin or b-globin (U.S. Pat. Nos. 8,278,063; 9,012,219), human cytochrome b-245 a polypeptide, and hydroxysteroid (17b) dehydrogenase, and Tobacco etch virus (U.S. Pat. Nos. 8,278,063, 9,012,219). CMV immediate-early 1 (IE1) gene (US2014/0206753, WO2013/185069), the sequence GGGAUCCUACC (WO2014/144196) may also be used. In another embodiment, 5′ UTR of a TOP gene is a 5′ UTR of a TOP gene lacking the 5′ TOP motif (the oligopyrimidine tract) (e.g., WO2015/101414, WO2015/101415, WO2015/062738, WO2015/024667, WO2015/024667; 5′ UTR element derived from ribosomal protein Large 32 (L32) gene (WO2015/101414, WO2015/101415, WO2015/062738), 5′ UTR element derived from the 5′UTR of an hydroxysteroid (17-β) dehydrogenase 4 gene (HSD17B4) (WO2015/024667), or a 5′ UTR element derived from the 5′ UTR of ATP5A1 (WO2015/024667) can be used. In some embodiments, an internal ribosome entry site (IRES) is used instead of a 5′ UTR.

A 3′ UTR is region of an mRNA that is directly downstream (3′) from the stop codon (the codon of an mRNA transcript that signals a termination of translation). A 3′ UTR does not encode a protein (is non-coding). Natural or wild type 3′ UTRs are known to have stretches of adenosines and uridines embedded in them. These AU rich signatures are particularly prevalent in genes with high rates of turnover. Based on their sequence features and functional properties, the AU rich elements (AREs) can be separated into three classes (Chen et al, 1995): Class I AREs contain several dispersed copies of an AUUUA motif within U-rich regions. C-Myc and MyoD contain class I AREs. Class II AREs possess two or more overlapping UUAUUUA(U/A)(U/A) nonamers. Molecules containing this type of AREs include GM-CSF and TNF-a. Class III ARES are less well defined. These U rich regions do not contain an AUUUA motif c-Jun and Myogenin are two well-studied examples of this class. Most proteins binding to the AREs are known to destabilize the messenger, whereas members of the ELAV family, most notably HuR, have been documented to increase the stability of mRNA. HuR binds to AREs of all the three classes. Engineering the HuR specific binding sites into the 3′ UTR of nucleic acid molecules will lead to HuR binding and thus, stabilization of the message in vivo.

Introduction, removal, or modification of 3′ UTR AU rich elements (AREs) can be used to modulate the stability of nucleic acids (e.g., RNA) of the disclosure. When engineering specific nucleic acids, one or more copies of an ARE can be introduced to make nucleic acids of the disclosure less stable and thereby curtail translation and decrease production of the resultant protein. Likewise, AREs can be identified and removed or mutated to increase the intracellular stability and thus increase translation and production of the resultant protein. Transfection experiments can be conducted in relevant cell lines, using nucleic acids of the disclosure and protein production can be assayed at various time points post-transfection. For example, cells can be transfected with different ARE-engineering molecules and by using an ELISA kit to the relevant protein and assaying protein produced at 6 hour, 12 hour, 24 hour, 48 hour, and 7 days post-transfection.

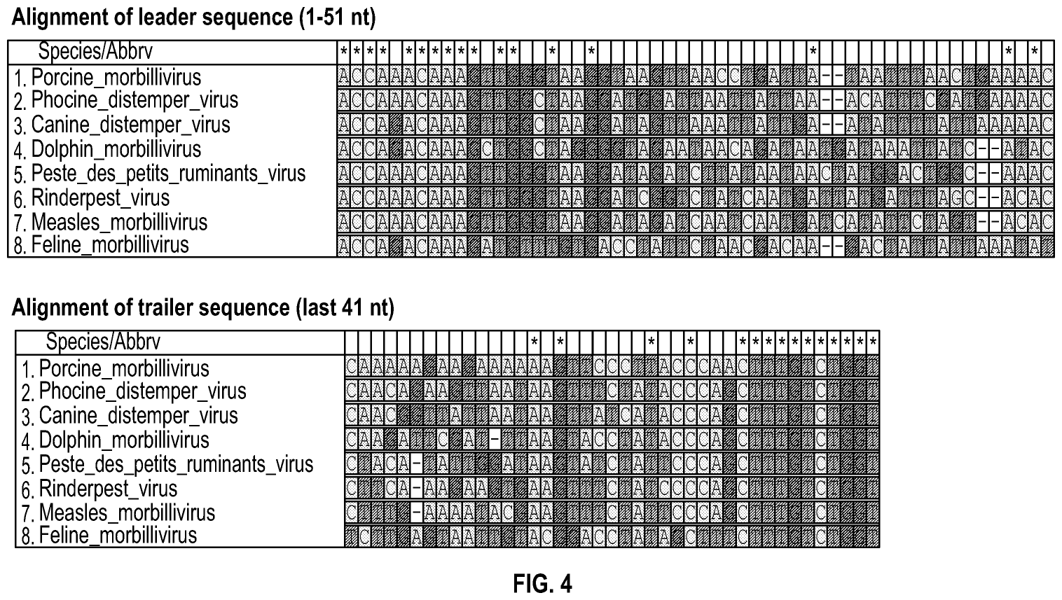

3′ UTRs may be heterologous or synthetic. With respect to 3′ UTRs, globin UTRs, including Xenopus β-globin UTRs and human β-globin UTRs are known in the art (U.S. Pat. Nos. 8,278,063, 9,012,219, US2011/0086907). A modified β-globin construct with enhanced stability in some cell types by cloning two sequential human β-globin 3′UTRs head to tail has been developed and is well known in the art (US2012/0195936, WO2014/071963). In addition a2-globin, al-globin, UTRs and mutants thereof are also known in the art (WO2015/101415, WO2015/024667). Other 3′ UTRs described in the mRNA constructs in the non-patent literature include CYBA (Ferizi et al., 2015) and albumin (Thess et al., 2015). Other exemplary 3′ UTRs include that of bovine or human growth hormone (wild type or modified) (WO2013/185069, US2014/0206753, WO2014/152774), rabbit 13 globin and hepatitis B virus (HBV), α-globin 3′ UTR and Viral VEEV 3′ UTR sequences are also known in the art. In some embodiments, the sequence UUUGAAUU (WO2014/144196) is used. In some embodiments, 3′ UTRs of human and mouse ribosomal protein are used. Other examples include rps9 3′UTR (WO2015/101414), FIG. 4 (WO2015/101415), and human albumin 7 (WO2015/101415).

Those of ordinary skill in the art will understand that 5′UTRs that are heterologous or synthetic may be used with any desired 3′ UTR sequence. For example, a heterologous 5′UTR may be used with a synthetic 3′UTR with a heterologous 3″ UTR.

Non-UTR sequences may also be used as regions or subregions within a nucleic acid. For example, introns or portions of introns sequences may be incorporated into regions of nucleic acid of the disclosure. Incorporation of intronic sequences may increase protein production as well as nucleic acid levels.

Combinations of features may be included in flanking regions and may be contained within other features. For example, the ORF may be flanked by a 5′ UTR which may contain a strong Kozak translational initiation signal and/or a 3′ UTR which may include an oligo(dT) sequence for templated addition of a poly-A tail. 5′ UTR may comprise a first polynucleotide fragment and a second polynucleotide fragment from the same and/or different genes such as the 5′ UTRs described in US Patent Application Publication No. 2010/0293625 and PCT/US2014/069155, herein incorporated by reference in its entirety.

It should be understood that any UTR from any gene may be incorporated into the regions of a nucleic acid. Furthermore, multiple wild-type UTRs of any known gene may be utilized. It is also within the scope of the present disclosure to provide artificial UTRs which are not variants of wild type regions. These UTRs or portions thereof may be placed in the same orientation as in the transcript from which they were selected or may be altered in orientation or location. Hence a 5′ or 3′ UTR may be inverted, shortened, lengthened, made with one or more other 5′ UTRs or 3′ UTRs. As used herein, the term “altered” as it relates to a UTR sequence, means that the UTR has been changed in some way in relation to a reference sequence. For example, a 3′ UTR or 5′ UTR may be altered relative to a wild-type or native UTR by the change in orientation or location as taught above or may be altered by the inclusion of additional nucleotides, deletion of nucleotides, swapping or transposition of nucleotides. Any of these changes producing an “altered” UTR (whether 3′ or 5′) comprise a variant UTR.

In some embodiments, a double, triple or quadruple UTR such as a 5′ UTR or 3′ UTR may be used. As used herein, a “double” UTR is one in which two copies of the same UTR are encoded either in series or substantially in series. For example, a double beta-globin 3′ UTR may be used as described in US Patent publication 20100129877, the contents of which are incorporated herein by reference in its entirety.

It is also within the scope of the present disclosure to have patterned UTRs. As used herein “patterned UTRs” are those UTRs which reflect a repeating or alternating pattern, such as ABABAB or AABBAABBAABB or ABCABCABC or variants thereof repeated once, twice, or more than 3 times. In these patterns, each letter, A, B, or C represent a different UTR at the nucleotide level.

In some embodiments, flanking regions are selected from a family of transcripts whose proteins share a common function, structure, feature or property. For example, polypeptides of interest may belong to a family of proteins which are expressed in a particular cell, tissue or at some time during development. The UTRs from any of these genes may be swapped for any other UTR of the same or different family of proteins to create a new polynucleotide. As used herein, a “family of proteins” is used in the broadest sense to refer to a group of two or more polypeptides of interest which share at least one function, structure, feature, localization, origin, or expression pattern.

The untranslated region may also include translation enhancer elements (TEE). As a non-limiting example, the TEE may include those described in US Application No. 2009/0226470, herein incorporated by reference in its entirety, and those known in the art.

cDNA encoding the polynucleotides described herein may be transcribed using an in vitro transcription (IVT) system. In vitro transcription of RNA is known in the art and is described in International Publication WO2014/152027, which is incorporated by reference herein in its entirety.

In some embodiments, the RNA transcript is generated using a non-amplified, linearized DNA template in an in vitro transcription reaction to generate the RNA transcript. In some embodiments, the template DNA is isolated DNA. In some embodiments, the template DNA is cDNA. In some embodiments, the cDNA is formed by reverse transcription of a RNA polynucleotide. In some embodiments, cells, e.g., bacterial cells, e.g., E. coli, e.g., DH-1 cells are transfected with the plasmid DNA template. In some embodiments, the transfected cells are cultured to replicate the plasmid DNA which is then isolated and purified. In some embodiments, the DNA template includes a RNA polymerase promoter, e.g., a T7 promoter located 5′ to and operably linked to the gene of interest.

In some embodiments, an in vitro transcription template encodes a 5′ untranslated (UTR) region, contains an open reading frame, and encodes a 3′ UTR and a polyA tail. The particular nucleic acid sequence composition and length of an in vitro transcription template will depend on the mRNA encoded by the template.

A “5′ untranslated region” (UTR) refers to a region of an mRNA that is directly upstream (i.e., 5′) from the start codon (i.e., the first codon of an mRNA transcript translated by a ribosome) that does not encode a polypeptide. When RNA transcripts are being generated, the 5′ UTR may comprise a promoter sequence. Such promoter sequences are known in the art. It should be understood that such promoter sequences will not be present in a vaccine of the disclosure.

A “3′ untranslated region” (UTR) refers to a region of an mRNA that is directly downstream (i.e., 3′) from the stop codon (i.e., the codon of an mRNA transcript that signals a termination of translation) that does not encode a polypeptide.

An “open reading frame” is a continuous stretch of DNA beginning with a start codon (e.g., methionine (ATG)), and ending with a stop codon (e.g., TAA, TAG or TGA) and encodes a polypeptide.

A “polyA tail” is a region of mRNA that is downstream, e.g., directly downstream (i.e., 3′), from the 3′ UTR that contains multiple, consecutive adenosine monophosphates. A polyA tail may contain 10 to 300 adenosine monophosphates. For example, a polyA tail may contain 10, 20, 30, 40, 50, 60, 70, 80, 90, 100, 110, 120, 130, 140, 150, 160, 170, 180, 190, 200, 210, 220, 230, 240, 250, 260, 270, 280, 290 or 300 adenosine monophosphates. In some embodiments, a polyA tail contains 50 to 250 adenosine monophosphates. In a relevant biological setting (e.g., in cells, in vivo) the poly(A) tail functions to protect mRNA from enzymatic degradation, e.g., in the cytoplasm, and aids in transcription termination, and/or export of the mRNA from the nucleus and translation.

In some embodiments, a nucleic acid includes 200 to 3,000 nucleotides. For example, a nucleic acid may include 200 to 500, 200 to 1000, 200 to 1500, 200 to 3000, 500 to 1000, 500 to 1500, 500 to 2000, 500 to 3000, 1000 to 1500, 1000 to 2000, 1000 to 3000, 1500 to 3000, or 2000 to 3000 nucleotides).

An in vitro transcription system typically comprises a transcription buffer, nucleotide triphosphates (NTPs), an RNase inhibitor and a polymerase.

The NTPs may be manufactured in house, may be selected from a supplier, or may be synthesized as described herein. The NTPs may be selected from, but are not limited to, those described herein including natural and unnatural (modified) NTPs.

Any number of RNA polymerases or variants may be used in the method of the present disclosure. The polymerase may be selected from, but is not limited to, a phage RNA polymerase, e.g., a T7 RNA polymerase, a T3 RNA polymerase, a SP6 RNA polymerase, and/or mutant polymerases such as, but not limited to, polymerases able to incorporate modified nucleic acids and/or modified nucleotides, including chemically modified nucleic acids and/or nucleotides. Some embodiments exclude the use of DNase.

In some embodiments, the RNA transcript is capped via enzymatic capping. In some embodiments, the RNA comprises 5′ terminal cap, for example, 7mG(5′)ppp(5′)NlmpNp.

Nucleic acids the present disclosure may be manufactured in whole or in part using solid phase techniques. Solid-phase chemical synthesis of nucleic acids is an automated method wherein molecules are immobilized on a solid support and synthesized step by step in a reactant solution. Solid-phase synthesis is useful in site-specific introduction of chemical modifications in the nucleic acid sequences.

The synthesis of nucleic acids of the present disclosure by the sequential addition of monomer building blocks may be carried out in a liquid phase.

The synthetic methods discussed above each has its own advantages and limitations. Attempts have been conducted to combine these methods to overcome the limitations. Such combinations of methods are within the scope of the present disclosure. The use of solid-phase or liquid-phase chemical synthesis in combination with enzymatic ligation provides an efficient way to generate long chain nucleic acids that cannot be obtained by chemical synthesis alone.

Assembling nucleic acids by a ligase may also be used. DNA or RNA ligases promote intermolecular ligation of the 5′ and 3′ ends of polynucleotide chains through the formation of a phosphodiester bond. Nucleic acids such as chimeric polynucleotides and/or circular nucleic acids may be prepared by ligation of one or more regions or subregions. DNA fragments can be joined by a ligase catalyzed reaction to create recombinant DNA with different functions. Two oligodeoxynucleotides, one with a 5′ phosphoryl group and another with a free 3′ hydroxyl group, serve as substrates for a DNA ligase.

Purification of the nucleic acids described herein may include, but is not limited to, nucleic acid clean-up, quality assurance and quality control. Clean-up may be performed by methods known in the arts such as, but not limited to, AGENCOURT® beads (Beckman Coulter Genomics, Danvers, Mass.), poly-T beads, LNATM oligo-T capture probes (EXIQON® Inc, Vedbaek, Denmark) or HPLC based purification methods such as, but not limited to, strong anion exchange HPLC, weak anion exchange HPLC, reverse phase HPLC (RP-HPLC), and hydrophobic interaction HPLC (HIC-HPLC). The term “purified” when used in relation to a nucleic acid such as a “purified nucleic acid” refers to one that is separated from at least one contaminant. A “contaminant” is any substance that makes another unfit, impure or inferior. Thus, a purified nucleic acid (e.g., DNA and RNA) is present in a form or setting different from that in which it is found in nature, or a form or setting different from that which existed prior to subjecting it to a treatment or purification method.

A quality assurance and/or quality control check may be conducted using methods such as, but not limited to, gel electrophoresis, UV absorbance, or analytical HPLC.

In some embodiments, the nucleic acids may be sequenced by methods including, but not limited to reverse-transcriptase-PCR.

In some embodiments, the nucleic acids of the present disclosure may be quantified in exosomes or when derived from one or more bodily fluid. Bodily fluids include peripheral blood, serum, plasma, ascites, urine, cerebrospinal fluid (CSF), sputum, saliva, bone marrow, synovial fluid, aqueous humor, amniotic fluid, cerumen, breast milk, broncheoalveolar lavage fluid, semen, prostatic fluid, cowper's fluid or pre-ejaculatory fluid, sweat, fecal matter, hair, tears, cyst fluid, pleural and peritoneal fluid, pericardial fluid, lymph, chyme, chyle, bile, interstitial fluid, menses, pus, sebum, vomit, vaginal secretions, mucosal secretion, stool water, pancreatic juice, lavage fluids from sinus cavities, bronchopulmonary aspirates, blastocyl cavity fluid, and umbilical cord blood. Alternatively, exosomes may be retrieved from an organ selected from the group consisting of lung, heart, pancreas, stomach, intestine, bladder, kidney, ovary, testis, skin, colon, breast, prostate, brain, esophagus, liver, and placenta.

Assays may be performed using construct specific probes, cytometry, qRT-PCR, real-time PCR, PCR, flow cytometry, electrophoresis, mass spectrometry, or combinations thereof while the exosomes may be isolated using immunohistochemical methods such as enzyme linked immunosorbent assay (ELISA) methods. Exosomes may also be isolated by size exclusion chromatography, density gradient centrifugation, differential centrifugation, nanomembrane ultrafiltration, immunoabsorbent capture, affinity purification, microfluidic separation, or combinations thereof.

These methods afford the investigator the ability to monitor, in real time, the level of nucleic acids remaining or delivered. This is possible because the nucleic acids of the present disclosure, in some embodiments, differ from the endogenous forms due to the structural or chemical modifications.

In some embodiments, the nucleic acid may be quantified using methods such as, but not limited to, ultraviolet visible spectroscopy (UV/Vis). A non-limiting example of a UV/Vis spectrometer is a NANODROP® spectrometer (ThermoFisher, Waltham, Mass.). The quantified nucleic acid may be analyzed in order to determine if the nucleic acid may be of proper size, check that no degradation of the nucleic acid has occurred. Degradation of the nucleic acid may be checked by methods such as, but not limited to, agarose gel electrophoresis, HPLC based purification methods such as, but not limited to, strong anion exchange HPLC, weak anion exchange HPLC, reverse phase HPLC (RP-HPLC), and hydrophobic interaction HPLC (HIC-HPLC), liquid chromatography-mass spectrometry (LCMS), capillary electrophoresis (CE) and capillary gel electrophoresis (CGE).

In some embodiments, RNA (e.g., mRNA) vaccines of the disclosure are formulated in a lipid nanoparticle (LNP). Lipid nanoparticles typically comprise ionizable cationic lipid, non-cationic lipid, sterol and PEG lipid components along with the nucleic acid cargo of interest. The lipid nanoparticles of the disclosure can be generated using components, compositions, and methods as are generally known in the art, see for example PCT/US2016/052352; PCT/US2016/068300; PCT/US2017/037551; PCT/US2015/027400; PCT/US2016/047406; PCT/US2016000129; PCT/US2016/014280; PCT/US2016/014280; PCT/US2017/038426; PCT/US2014/027077; PCT/US2014/055394; PCT/US2016/52117; PCT/US2012/069610; PCT/US2017/027492; PCT/US2016/059575 PCT/US2016/069491, and US201762473219, all of which are incorporated by reference herein in their entirety.

The vaccines, as provided herein, may include an RNA or multiple RNAs encoding two or more antigens of the same or different viral strains. Also provided herein are combination vaccines that include RNA encoding one or more antigen(s) and one or more antigen(s) of a different organisms (e.g., bacterial and/or viral organism). Thus, the vaccines of the present disclosure may be combination vaccines that target one or more antigens of the same strain/species, or one or more antigens of different strains/species, e.g., antigens which induce immunity to organisms which are found in the same geographic areas where the risk of PoMV infection is high or organisms to which an individual is likely to be exposed to when exposed to the virus.

### Artificial Antigen-Presenting Cells Vaccines

Artificial antigen-presenting cells, aAPGs, are compositions that mimic antigen-presenting cells, such as dendritic cells. aAPGs may be used to expand a desired T cell, activate and/or expand specific T cell subsets, identify stimulatory molecules, co-stimulatory molecules, and combinations thereof, that can promote expansion of specific T cell subsets, as well as numerous therapeutic uses relating to expansion and stimulation of T cells. aAPGs may be either a bead-based system or lentivirus transformed host cells.

Bead-based systems are well known in the art and any bead known in the art may be coated with a PoMV antigen (see, e.g., Levine et al., 1996, Science 272:1939-1943; Riley et al., 1997, J. Immunol. 158:5545-5553 Carroll et al., 1997, Science 276:273-276; Carroll et al., 1997, Science 276:273-276; Levine et al., 2002, Nature Med. 8:47-53; Walker et al., 2000, Blood 96:467-474; Ranga et al., 1998, Proc. Natl. Acad. Sci. USA 95:1201-1206; Levine et al., 2002, Nature Med. 8:47-53; Walker et al., 2000, Blood 96:467-474; Mitsuyasu et al., 2000, Blood 96:785-793; Decks et al., 2002, Mol. Ther. 5:788-797, Rosenberg et al., 1990, N. Engl. J. Med. 323:570-578; Yee et al., 2002, Proc. Natl. Acad. Sci. U.S.A. 99:16168-16173; Brodie et al., 1999, Nature Med. 5:34-41; Riddell et al., 1996, Nature Med. 2:216-223; Riddell et al., 2000, Cancer Journal 6:S250-S258).

Newer aAPCs comprise a host cells, for example K562 cells, transduced using a lentiviral vector (LV), wherein said LV comprises a nucleic acid encoding at least one immune stimulatory ligand and at least one co-stimulatory ligand. These aAPCs may then stimulate and expand desired T cells against the antigen encoded by the LV. Any molecule or ligand, whether stimulatory, co-stimulatory, cytokine, antigen, Fey receptor, and the like, can be introduced into these cells to produce an aAPC of the invention.

There is extensive knowledge in the art regarding the events and molecules involved in activation and induction of T cell, and treatises discussing T cell mediated immune responses, and the factors mediating them, are well-known in the art. Further, the extensive disclosure provided in WO 03/057171 and US2003/0147869 is incorporated by reference as if set forth in its entirety herein. More specifically, a primary signal, usually mediated via the T cell receptor/CD3 complex on a T cell, initiates the T cell activation process. Additionally, numerous co-stimulatory molecules present on the surface of a T cell are involved in regulating the transition from resting T cell to cell proliferation. Such co-stimulatory molecules, also referred to as “co-stimulators”, which specifically bind with their respective ligands, include, but are not limited to, CD28 (which binds with B7-1 [CD80], B7-2 [CD86]), PD-1. (which binds with ligands PD-L1 and PD-L2), B7-113, 4-IBB (binds the ligand 4-1BBL), OX40 (binds ligand OX40L), ICOS (binds ligand ICOS-L), and LFA (binds the ligand ICAM). Thus, the primary stimulatory signal mediates T cell stimulation, but the co-stimulatory signal is then required for T cell activation, as demonstrated by proliferation.

Thus, the aAPC of the invention encompasses a cell comprising a stimulatory ligand that specifically binds with a TCR/CD3 complex such that a primary signal is transduced. Additionally, as would be appreciated by one skilled in the art, based upon the disclosure provided herein, the aAPC further comprises at least one co-stimulatory ligand that specifically binds with at least one co-stimulatory molecule present on a T cell, which co-stimulatory molecule includes, but is not limited to, CD27, CD28, CD30, CD7, a ligand that specifically binds with CD83, 4-IBB, PD-1, OX40, ICOS, LFA-1, CD30L, NKG2C, B7-H3, MHC class I, BTLA, Toll ligand receptor and LIGHT. This is because a co-stimulatory signal is required to induce T cell activation and associated proliferation. Other co-stimulatory ligands are encompassed in the invention, as would be understood by one skilled in the art armed with the teachings provided herein. Such ligands include, but are not limited to, a mutant, a variant, a fragment and a homolog of the natural ligands described previously.

These and other ligands are well-known in the art and have been well characterized as described in, e.g., Schwartz et al., 2001, Nature 410:604-608; Schwartz et al., 2002, Nature Immunol, 3:427-434; and Zhang et al., 2004, Immunity, 20:337-347, Using the extensive knowledge in the art concerning the ligand, the skilled artisan, armed with the teachings provided herein would appreciate that a mutant or variant of the ligand is encompassed in the invention and can be transduced into a cell using a LV to produce the aAPC of the invention and such mutants and variants are discussed more fully elsewhere herein. That is, the invention includes using a mutant or variant of a ligand of interest and methods of producing such mutants and variants are well-known in the art and are not discussed further herein.

Thus, the aAPC of the invention comprises at least one stimulatory ligand, for example a PoMV antigen, and at least one co-stimulatory ligand, such that the aAPC can stimulate and expand a T cell comprising a cognate binding partner stimulatory molecule that specifically binds with the stimulatory ligand on the aAPC and a cognate binding partner co-stimulatory molecule that specifically binds with the co-stimulatory ligand on the aAPC such that interaction between the ligands on the aAPC and the corresponding molecules on the T cell mediate, among other things, T cell proliferation, expansion and immune response as desired. One skilled in the art would appreciate that where the particular stimulatory and co-stimulatory molecules on a T cell of interest are known, an aAPC of the invention can be readily produced to expand that T cell. Conversely, where the stimulatory and co-stimulatory molecules on a T cell of interest are not known, a panel of aAPCs of the invention can be used to determine which molecules, and combinations thereof, can expand that T cell. Thus, the present invention provides tools for expansion of desirable T cells, as well as tools for elucidating the molecules on particular T cells that mediate T cell activation and proliferation.

The skilled artisan would understand that the nucleic acids of the invention encompass an RNA or a DNA sequence encoding a protein of the invention, and any modified forms thereof, including chemical modifications of the DNA or RNA which render the nucleotide sequence more stable when it is cell free or when it is associated with a cell. Chemical modifications of nucleotides may also be used to enhance the efficiency with which a nucleotide sequence is taken up by a cell or the efficiency with which it is expressed in a cell. Any and all combinations of modifications of the nucleotide sequences are contemplated in the present invention.

Further, any number of procedures may be used for the generation of mutant, derivative or variant forms of a protein of the invention using recombinant DNA methodology well known in the art and discussed herein.

The present invention also provides for analogs of proteins or peptides which comprise a costimulatory ligand as disclosed herein. Analogs may differ from naturally occurring proteins or peptides by conservative amino acid sequence differences or by modifications which do not affect sequence, or by both. Modifications (which do not normally alter primary sequence) include in vivo, or in vitro, chemical derivatization of polypeptides, e.g., acetylation, or carboxylation. Also included are modifications of glycosylation, e.g., those made by modifying the glycosylation patterns of a polypeptide during its synthesis and processing or in further processing steps; e.g., by exposing the polypeptide to enzymes which affect glycosylation, e.g., mammalian glycosylating or deglycosylating enzymes. Also embraced are sequences which have phosphorylated amino acid residues, e.g., phosphotyrosine, phosphoserine, or phosphothreonine.

Also included are polypeptides which have been modified using ordinary molecular biological techniques so as to improve their resistance to proteolytic degradation or to optimize solubility properties or to render them more suitable as a therapeutic agent. Analogs of such polypeptides include those containing residues other than naturally occurring L-amino acids, e.g., D-amino acids or non-naturally occurring synthetic amino acids. The peptides of the invention are not limited to products of any of the specific exemplary processes listed herein.

Among a “biological activity”, as used herein, is included a costimulatory ligand which when transduced into a host cell, for example a K562 cell, is expressed and, when the cell is contacted with a T cell expressing a cognate costimulatory molecule on its surface, it mediates activation and stimulation of the T cell, with induced proliferation.

The invention encompasses an aAPC comprising a nucleic acid encoding a PoMV antigen. An antigen of interest can be introduced into an aAPC of the invention, wherein the aAPC then presents the antigen in the context of the MCH Class I or II complex, i.e., the MHC molecule is “loaded” with the antigen, and the aAPC can be used to produce an antigen-specific T cell.

Additionally, the invention encompasses an aAPC transduced with a nucleic acid encoding at least one cytokine, at least one chemokine, tag polypeptide, or a combination thereof. See U.S. Pat. No. 10,286,066, herein incorporated in its entirety.

### Antigen Vaccine

The vaccines of the present invention comprise an immunogenic polypeptide or a recombinant protein comprising a multi-epitope polypeptide, and optionally, an adjuvant. The vaccine can be formulated for administration in one of many different modes. In some embodiments the vaccine is formulated for mass inoculation, for example for use with a jet-injector or a single use cartridge. Needles specifically designed to deposit the vaccine intradermally are known in the art as disclosed for example in U.S. Pat. Nos. 6,843,781 and 7,250,036 among others. According to other embodiments the administration is performed with a needleless injector.

Liposomes provide another delivery system for antigen delivery and presentation. Liposomes are bilayered vesicles composed of phospholipids and other sterols surrounding a typically aqueous center where antigens or other products can be encapsulated. The liposome structure is highly versatile with many types range in nanometer to micrometer sizes, from about 25 nm to about 50 μm. Liposomes have been found to be effective in delivering therapeutic agents to dermal and mucosal surfaces. Liposomes can be further modified for targeted delivery by for example, incorporating specific antibodies into the surface membrane, or altered to encapsulate bacteria, viruses, or parasites. The average survival time or half-life of the intact liposome structure can be extended with the inclusion of certain polymers, for example polyethylene glycol, allowing for prolonged release in vivo. Liposomes may be unilamellar or multilamellar.

The vaccine composition may be formulated by: encapsulating an antigen or an antigen/adjuvant complex in liposomes to form liposome-encapsulated antigen and mixing the liposome-encapsulated antigen with a carrier comprising a continuous phase of a hydrophobic substance. If an antigen/adjuvant complex is not used in the first step, a suitable adjuvant may be added to the liposome-encapsulated antigen, to the mixture of liposome-encapsulated antigen and carrier, or to the carrier before the carrier is mixed with the liposome-encapsulated antigen. The order of the process may depend on the type of adjuvant used. Typically, when an adjuvant like alum is used, the adjuvant and the antigen are mixed first to form an antigen/adjuvant complex followed by encapsulation of the antigen/adjuvant complex with liposomes. The resulting liposome-encapsulated antigen is then mixed with the carrier. The term “liposome-encapsulated antigen” may refer to encapsulation of the antigen alone or to the encapsulation of the antigen/adjuvant complex depending on the context. This promotes intimate contact between the adjuvant and the antigen and may, at least in part, account for the immune response when alum is used as the adjuvant. When another is used, the antigen may be first encapsulated in liposomes and the resulting liposome-encapsulated antigen is then mixed into the adjuvant in a hydrophobic substance.

In formulating a vaccine composition that is substantially free of water, antigen or antigen/adjuvant complex is encapsulated with liposomes and mixed with a hydrophobic substance. In formulating a vaccine in an emulsion of water-in-a hydrophobic substance, the antigen or antigen/adjuvant complex is encapsulated with liposomes in an aqueous medium followed by the mixing of the aqueous medium with a hydrophobic substance. In the case of the emulsion, to maintain the hydrophobic substance in the continuous phase, the aqueous medium containing the liposomes may be added in aliquots with mixing to the hydrophobic substance.

In all methods of formulation, the liposome-encapsulated antigen may be freeze-dried before being mixed with the hydrophobic substance or with the aqueous medium as the case may be. In some instances, an antigen/adjuvant complex may be encapsulated by liposomes followed by freeze-drying. In other instances, the antigen may be encapsulated by liposomes followed by the addition of adjuvant then freeze-drying to form a freeze-dried liposome-encapsulated antigen with external adjuvant. In yet another instance, the antigen may be encapsulated by liposomes followed by freeze-drying before the addition of adjuvant. Freeze-drying may promote better interaction between the adjuvant and the antigen resulting in a more efficacious vaccine.

Formulation of the liposome-encapsulated antigen into a hydrophobic substance may also involve the use of an emulsifier to promote more even distribution of the liposomes in the hydrophobic substance. Typical emulsifiers are well-known in the art and include mannide oleate (Arlacel™ A), lecithin, Tween™ 80, Spans™ 20, 80, 83 and 85. The emulsifier is used in an amount effective to promote even distribution of the liposomes. Typically, the volume ratio (v/v) of hydrophobic substance to emulsifier is in the range of about 5:1 to about 15:1.

Microparticles and nanoparticles employ small biodegradable spheres which act as depots for vaccine delivery. The major advantage that polymer microspheres possess over other depot-effecting adjuvants is that they are extremely safe and have been approved by the Food and Drug Administration in the US for use in human medicine as suitable sutures and for use as a biodegradable drug, delivery system (Langer R. Science. 1990, 249, 1527). The rates of copolymer hydrolysis are very well characterized, which in turn allows for the manufacture of microparticles with sustained antigen release over prolonged periods of time (O'Hagen, et al., Vaccine. 1993, 11, 965).

Parenteral administration of microparticles may elicit long-lasting immunity, especially if they incorporate prolonged release characteristics. The rate of release can be modulated by the mixture of polymers and their relative molecular weights, which will hydrolyze over varying periods of time. Without wishing to be bound to theory, the formulation of different sized particles (1 μm to 200 μm) may also contribute to long-lasting immunological responses since large particles must be broken down into smaller particles before being available for macrophage uptake. In this manner a single-injection vaccine could be developed by integrating various particle sizes, thereby prolonging antigen presentation and greatly benefiting livestock producers.

In some applications an adjuvant or excipient may be included in the vaccine formulation. Montanide™ and alum for example, are preferred adjuvants for human use. The choice of the adjuvant will be determined in part by the mode of administration of the vaccine. For example, non-injected vaccination will lead to better overall compliance and lower overall costs. A preferred mode of administration is intramuscular administration. Another preferred mode of administration is intranasal administration. Non-limiting examples of intranasal adjuvants include chitosan powder, PLA and PLG microspheres, QS-21, calcium phosphate nanoparticles (CAP) and mCTA/LTB (mutant cholera toxin. E112K with pentameric B subunit of heat labile enterotoxin).

According to several embodiments, the vaccine compositions according to the present invention may contain one or more adjuvants, characterized in that it is present as a solution or emulsion which is substantially free from inorganic salt ions, wherein said solution or emulsion contains one or more water soluble or water-emulsifiable substances which is capable of making the vaccine isotonic or hypotonic. The water soluble or water-emulsifiable substances may be, for example, selected from the group consisting of: maltose; fructose; galactose; saccharose; sugar alcohol; lipid; and combinations thereof.

The formulations of the present invention may optionally comprise a mucosal delivery-enhancing agent such as for example a permeabilizing peptide that reversibly enhances mucosal epithelial paracellular transport by modulating epithelial junctional structure and/or physiology, as described in US 2004/0077540.

The immunogenic polypeptides used in the methods and compositions of the present invention may comprise in other exemplary embodiments a proteosome adjuvant. The proteosome adjuvant comprises a purified preparation of outer membrane proteins of meningococci and similar preparations from other bacteria. These proteins are highly hydrophobic, reflecting their role as transmembrane proteins and porins. Due to their hydrophobic protein-protein interactions, when appropriately isolated, the proteins form multi-molecular structures consisting of about 60-100 nm diameter whole or fragmented membrane vesicles. This liposome-like physical state allows the proteosome adjuvant to act as a protein carrier and also to act as an adjuvant. Polypeptides used according to the present invention are optionally complexed to the proteosome antigen vesicles through hydrophobic moieties. For example, an antigen is conjugated to a lipid moiety such as a fatty acyl group. Such a hydrophobic moiety may be linked directly to the HA3 immunogenic polypeptide or alternatively, a short spacer, for example, of one, two, three or four, up to six or ten amino acids can be used to link the polypeptide to the fatty group. This hydrophobic anchor interacts with the hydrophobic membrane of the proteosome adjuvant vesicles, while presenting the generally hydrophilic antigenic peptide.

In particular, a hydrophobic anchor may comprise a fatty acyl group attached to the amino terminus or near the carboxyl terminus of the immunogenic polypeptide. One example is the twelve-carbon chain lauroyl (CH₃(CH)₁₀CO), although any similarly serving fatty acyl group including, but not limited to, acyl groups that are of eight-, ten-, fourteen-, sixteen-, eighteen-, or twenty-carbon chain lengths can also serve as hydrophobic anchors. The anchor may be linked to the peptide antigen using an immunopotentiating spacer. Such a linker may consist of 1-10 amino acids, which may assist in maintaining the conformational structure of the peptide.

The antigen content is best defined by the biological effect it provokes. Sufficient antigen should be present to provoke the production of measurable amounts of protective antibody. A convenient test for the biological activity of viruses involves the ability of the antigenic material undergoing testing to deplete a known positive antiserum of its protective antibody. The result is reported in the negative log of the LD50 (lethal dose, 50%) for mice treated with virulent organisms which are pretreated with a known antiserum which itself was pretreated with various dilutions of the antigenic material being evaluated. A high value is therefore reflective of a high content of antigenic material which has tied up the antibodies in the known antiserum thus reducing or eliminating the effect of the antiserum on the virulent organism making a small dose lethal. It is preferred that the antigenic material present in the final formulation is at a level sufficient to increase the negative log of LD50 by at least 1 preferably 1.4 compared to the result from the virulent organism treated with untreated antiserum. The absolute values obtained for the antiserum control and suitable vaccine material are, of course, dependent on the virulent organism and antiserum standards selected.

### Vaccine Formulations

Formulations for each of the above vaccines are known in the art (see for example US Pat. No. 10, 286,066 for aAPCs; US 2020/0261565 for viral vectors; and US 2020/0030432 for nucleic acid vaccines, all herein incorporated by reference in their entireties).

The following method may be also used to achieve a preferred vaccine formulation: starting from a defined antigen, which is intended to provoke the desired immune response, in a first step an adjuvant matched to the antigen is found, as described in the specialist literature, particularly in WO 97/30721. In a next step the vaccine is optimized by adding various isotonic-making substances as defined in the present inventions, preferably sugars and/or sugar alcohols, in an isotonic or slightly hypotonic concentration, to the mixture of antigen and adjuvant, with the composition otherwise being identical, and adjusting the solution to a physiological pH in the range from pH 4.0 to 10.0, particularly 7.4. Then, in a first step the substances or the concentration thereof which will improve the solubility of the antigen/adjuvant composition compared with a conventional, saline-buffered solution are determined. The improvement in the solubility characteristics by a candidate substance is a first indication that this substance is capable of bringing about an increase in the immunogenic activity of the vaccine.

Since one of the possible prerequisites for an increase in the cellular immune response is increased binding of the antigen to APCs (antigen presenting cells), in a next step an investigation can be made to see whether the substance leads to an increase of this kind. The procedure used may be analogous to that described in the definition of the adjuvant, e.g. incubating APCs with fluorescence-labelled peptide or protein, adjuvant and isotonic-making substance. An increased uptake or binding of the peptide to APCs brought about by the substance can be determined by comparison with cells which have been mixed with peptide and adjuvant alone or with a peptide/adjuvant composition which is present in conventional saline buffer solution, using throughflow cytometry.

In a second step the candidate substances may be investigated in vitro to see whether and to what extent their presence is able to increase the presentation of a peptide to APCs; the MHC concentration on the cells may be measured using the methods described in WO 97/30721 for testing peptides.

Another possible way of testing the efficiency of a formulation is by using an in vitro model system. In this, APCs are incubated together with adjuvant, peptide and candidate substance and the relative activation of a T-cell clone which specifically recognizes the peptide used is measured (Coligan et al., 1991; Lopez et al., 1993).

The efficiency of the formulation may optionally also be demonstrated by the cellular immune response by detecting a “delayed-type hypersensitivity” (DTH) reaction in immunized animals. Finally, the immunomodulatory activity of the formulation is measured in animal tests.

### Methods of Vaccinating a Subject

In another aspect, the present invention involves method of vaccinating a subject for PoMV comprising: administering the disclosed vaccine to a subject in need thereof. The disclosed vaccine may be administered in a number of ways. For example, the disclosed vaccine can be administered intramuscularly, intranasally, or by microneedle in the skin. The compositions may be administered orally, intravenously, subcutaneously, transdermally (e.g., by microneedle), intraperitoneally, ophthalmically, vaginally, rectally, sublingually, or by inhalation.

Parenteral administration of the composition, if used, is generally characterized by injection. Injectables can be prepared in conventional forms, either as liquid solutions or suspensions, solid forms suitable for solution of suspension in liquid prior to injection, or as emulsions. A revised approach for parenteral administration involves use of a slow release or sustained release system such that a constant dosage is maintained.

The exact amount of the compositions required will vary from subject to subject and from vaccine type to vaccine type, depending on the species, age, weight and general condition of the subject, the severity of the allergic disorder being treated, the particular nucleic acid or vector used, its mode of administration and the like. Thus, it is not possible to specify an exact amount for every composition. However, an appropriate amount can be determined by one of ordinary skill in the art using only routine experimentation given the teachings herein. For example, effective dosages and schedules for administering the compositions may be determined empirically, and making such determinations is within the skill in the art. The dosage ranges for the administration of the compositions are those large enough to produce the desired effect in which the symptoms disorder are affected. The dosage should not be so large as to cause adverse side effects, such as unwanted cross-reactions, anaphylactic reactions, and the like.

Generally, the dosage will vary with the age, condition, sex and extent of the disease in the patient, route of administration, or whether other drugs are included in the regimen, and can be determined by one of skill in the art. The dosage can be adjusted by the individual physician in the event of any counter indications. Dosage can vary, and can be administered in one or more dose administrations daily, for one or several days. Guidance can be found in the literature for appropriate dosages for given classes of pharmaceutical products. One skilled in the art can determine the proper dosage, for example a typical dosage of the disclosed vaccine used alone might range from about 0.1 μg/kg to up to about 100 mg/kg of body weight or more per vaccination, such as about 10 μg/kg to about 50 mg/kg, or about 50 μg/kg to about 10 mg/kg, depending on the factors mentioned above.

### Methods of Detecting PoMV in a Sample

In the present invention, a sample of genetic material and/or protein is obtained from an animal. Samples can be obtained from blood, tissue, semen, oral fluid, nasal wipes, nasal swabs, oropharyngeal swabs, urine, etc. Generally, tissue is used as the source, and the genetic material is RNA. A sufficient amount of cells are obtained to provide a sufficient amount for analysis. This amount will be known or readily determinable by those skilled in the art. The RNA and/or protein is isolated from the sample by techniques known to those skilled in the art.

The term “semiquantitative PCR” refers to a kind of polymerase chain reaction (PCR), which can be carried out on tissue samples, on serum and plasma using specific primers without a probe, and the term “qPCR” or “QPCR” refers to quantitative PCR, which can be carried out on tissue samples using specific primers and a dye which may be conjugated to a probe. In controlled reactions, the amount of product formed in a PCR reaction correlates with the amount of starting template (Sambrook, J., E Fritsch, E. and T Maniatis, Molecular Cloning: A Laboratory Manual 3^{rd} Cold Spring Harbor Laboratory Press: Cold Spring Harbor (2001). In semiquantitative PCR, quantification of the PCR product can be carried out by stopping the PCR reaction when it is in log phase before reagents become limiting. The PCR products are then electrophoresed in agarose or polyacrylamide gels, stained with ethidium bromide or a comparable DNA stain, such as Sybr Green, and the intensity of staining measured by densitometry. In qPCR, the progression of a PCR reaction can be measured in real time using PCR machines such as the Applied Biosystems' Prism 7000 or the Roche LightCycler which measure product accumulation in real-time. Real-time PCR measures either the fluorescence of DNA intercalating reporter dyes such as Sybr Green into the synthesized PCR product, or the fluorescence released by a reporter dye, such as, but not limited to cy3, cy5, FAM, SYBR Green, HEX™, JOE, TAMRA, Tye™ 563, TEX 615™, Tye™ 665, VIC, and/or LC Red 640, when cleaved from a quencher molecule, where the quencher molecule prevents the reporter dye from being detectable; the reporter and quencher molecules are incorporated into an oligonucleotide probe which hybridizes to the target DNA molecule following DNA strand extension from the primer oligonucleotides. The oligonucleotide probe is displaced and degraded by the enzymatic action of the DNA polymerase in the next PCR cycle, releasing the reporter from the quencher molecule, allowing the reporter dye to be detectable. In one variation, known as Scorpion™, the probe is covalently linked to the primer.

In some embodiments, reverse Transcription PCR (RT-PCR) can be used to compare RNA levels in different sample populations, in tissues, with or without drug treatment, to characterize patterns of expression, to discriminate between closely related RNAs, and to analyze RNA structure.

For RT-PCR, the first step is the isolation of RNA from a target sample. The starting material is typically total RNA isolated from an animal. The first step in gene expression profiling by RT-PCR is the reverse transcription of the RNA template into cDNA, followed by its exponential amplification in a PCR reaction. The two most commonly used reverse transcriptases are avian myeloblastosis virus reverse transcriptase (AMV-RT) and Moloney murine leukemia virus reverse transcriptase (MMLV-RT). The reverse transcription step is typically primed using specific primers, random hexamers, or oligo-dT primers, depending on the circumstances and the goal of expression profiling. For example, extracted RNA can be reverse-transcribed using a GeneAmp RNA PCR kit (Perkin Elmer, CA, USA), following the manufacturer's instructions. The derived cDNA can then be used as a template in the subsequent PCR reaction.

Although the PCR step can use a variety of thermostable DNA-dependent DNA polymerases, it typically employs the Taq DNA polymerase, which has a 5′-3′ nuclease activity but lacks a 3′-5′ proofreading endonuclease activity. Thus, TaqMan qPCR typically utilizes the 5′ nuclease activity of Taq or Tth polymerase to hydrolyze a hybridization probe bound to its target amplicon, but any enzyme with equivalent 5′ nuclease activity can be used.

In some embodiments, two oligonucleotide primers are used to generate an amplicon typical of a PCR reaction. A third oligonucleotide, or probe, used in real-time qPCR is designed to detect nucleotide sequence located between the two PCR primers. The probe is non-extendible by Taq DNA polymerase enzyme and is labeled with a reporter fluorescent dye and a quencher fluorescent dye. Any laser-induced emission from the reporter dye is quenched by the quenching dye when the two dyes are located close together as they are on the probe. During the amplification reaction, the Taq DNA polymerase enzyme cleaves the probe in a template-dependent manner. The resultant probe fragments disassociate in solution, and signal from the released reporter dye is free from the quenching effect of the second fluorophore. One molecule of reporter dye is liberated for each new molecule synthesized, and detection of the unquenched reporter dye provides the basis for quantitative interpretation of the data.

RT-PCR can be performed using commercially available equipment, such as, for example, ABI PRISM 7700 Sequence Detection System (Perkin-Elmer-Applied Biosystems, Foster. City, Calif., USA), or Lightcycler (Roche Molecular Biochemicals, Mannheim, Germany). In a preferred embodiment, the 5′ nuclease procedure is run on a real-time quantitative PCR device such as the ABI PRISM 7700 Sequence Detection System. The system consists of a thermocycler, laser, charge-coupled device (CCD), camera, and computer. The system amplifies samples in a multi-well format on a thermocycler. During amplification, laser-induced fluorescent signal is collected in real-time through fibre optics cables for each well, and detected at the CCD. The system includes software for running the instrument and for analyzing the data.

In some embodiments, 5′ nuclease assay data are initially expressed as Ct, or the threshold cycle. As discussed above, fluorescence values are recorded during every cycle and represent the amount of product amplified to that point in the amplification reaction. The point when the fluorescent signal is first recorded as statistically significant is the threshold cycle.

Real-Time Quantitative PCR (qRT-PCR) is a more recent variation of the RT-PCR technique is the real time quantitative PCR, which measures PCR product accumulation through a dual-labeled fluorogenic probe (i.e., TaqMan probe). Real time PCR is compatible both with quantitative competitive PCR and with quantitative comparative PCR. The former uses an internal competitor for each target sequence for normalization, while the latter uses a normalization gene contained within the sample, or a housekeeping gene for RT-PCR. Further details are provided, e.g., by Held et al., Genome Research 6: 986-994 (1996). PCR primers are designed to flank intron sequences present in the gene to be amplified. In this embodiment, the first step in the primer/probe design is the delineation of intron sequences within the genes. This can be done by publicly available software, such as the DNA BLAT software developed by Kent, W. J., Genome Res. 12 (4): 656-64 (2002), or by the BLAST software including its variations. Subsequent steps follow well established methods of PCR primer and probe design.

To avoid non-specific signals, it is useful to mask repetitive sequences within the introns when designing the primers and probes. This can be easily accomplished by using the Repeat Masker program available on-line through the Baylor College of Medicine, which screens DNA sequences against a library of repetitive elements and returns a query sequence in which the repetitive elements are masked. The masked sequences can then be used to design primer and probe sequences using any commercially or otherwise publicly available primer/probe design packages, such as Primer Express (Applied Biosystems); MGB assay-by-design (Applied Biosystems); Primer3 (Steve Rozen and Helen J. Skaletsky (2000) Primer3 on the VIMNV for general users and for biologist programmers in: Krawetz S, Misener S (eds) Bioinformatics Methods and Protocols: Methods in Molecular Biology. Humana Press, Totowa, N.J., pp 365-386).

The most important factors considered in PCR primer design include primer length, melting temperature (Tm), and G/C content, specificity, complementary primer sequences, and 3′ end sequence. In general, optimal PCR primers are generally 17-30 bases in length, and contain about 20-80% G+C bases, such as, for example, about 50-60% G+C bases. Melting temperatures between 50 and 80° C., e.g., about 50 to 70° C., are typically preferred. For further guidelines for PCR primer and probe design see, e.g., Dieffenbach, C. W. et al., General Concepts for PCR Primer Design in: PCR Primer, A Laboratory Manual, Cold Spring Harbor Laboratory Press, New York, 1995, pp. 133-155; Innis and Gelfand, Optimization of PCRs in: PCR Protocols, A Guide to Methods and Applications, CRC Press, London, 1994, pp. 5-11; and Plasterer, T. N. Primerselect: Primer and probe design. Methods Mol. Biol. 70: 520-527 (1997), the entire disclosures of which are hereby expressly incorporated by reference.

Enzyme-linked immunosorbent assay (ELISA) and sandwich ELISA are immunoassays that are advantageously used in the methods disclosed herein. In a (direct) ELISA, for example, an unknown amount of PoMV antigen is affixed to a substrate, and then a specific antibody is applied over the surface so that it can bind to the antigen. This antibody is conjugated to a reporter, such as, but not limited to, alkaline phosphatase, peroxidase, 0-galactosidase, Atto 425, Atto 488, Cy2, DyLight 405, DyLight 488, Atto 432 Atto 550, Cy3, Cy5, DyLight 549, TEX 615™, Allophycocyanin, Atto 647, DyLight 649, Atto 655, Cy5.5, Dylight 680, and/or DyLight 800, and, in the case of an enzyme being the conjugate, an enzyme, and in the final step a substance is added so that the enzyme can convert to some detectable signal, most commonly a color change in a chemical substrate. In a sandwich ELISA a capture antibody that can bind to the antigen is affixed to the substrate. The other steps are equivalent to the ELISA. The detectable signal can be detected by a number of commercially available systems, such as, but not limited to, the Bio-Rad iMark Readers, BioTek Synergy Readers (Winooski, VT), or various BMG LABTECH star Readers (Cary, NC).

In an Enzyme Immuno Assay (EIA), which is similar to the sandwich ELISA, streptavidin is affixed to a surface and then the capture antibody is biotinylated, otherwise the other steps are performed equivalently as the ELISA. The EIA immunoassay is advantageously used in the methods disclosed herein. The detectable signal can be detected by several commercially available systems, such as, but not limited to, the Bio-Rad iMark Readers, BioTek Synergy Readers (Winooski, VT), or various BMG LABTECH star Readers (Cary, NC).

In a blotting assay, such as Western Blot, the sample is separated out in an appropriate gel and then transferred to a substrate, such as, but not limited to, nitrocellulose or PVDF. The membrane is then blocked to prevent nonspecific protein binding, followed by incubating the blot with antigen specific antibodies. The antigen specific antibodies can be conjugated with a reporter, such as, but not limited to, alkaline phosphatase, peroxidase, 0-galactosidase, Atto 425, Atto 488, Cy2, DyLight 405, DyLight 488, Atto 432 Atto 550, Cy3, Cy5, DyLight 549, TEX 615™, Allophycocyanin, Atto 647, DyLight 649, Atto 655, Cy5.5, Dylight 680, and/or DyLight 800, or can be further bound by a secondary antibody conjugated to a reporter that can bind to the antigen specific antibody. Like in an ELISA, the blot can either be read directly if a reporter dye is conjugated or if an enzyme is conjugated, the appropriate substance is added to the blot in order to create a detectable signal. The signal can them be detected by a number of commercially available systems, such as, but not limited to, the Bio-Rad ChemiDoc (Hercules, CA) or a Typhoon Biomolecular Imager (GE Healthcare Life Sciences, Malborough, MA).

One of ordinary skill in the art will also understand that PoMV or a fragment thereof can also be part of high throughput assays. These high throughput assays may not be specifically designed to assay only PoMV or a fragment thereof, but rather the expression of multiple RNAs, cDNAs, or proteins at once. Example of such high throughput assays include, but are not limited to, spotted oligonucleotide microarrays, printed oligonucleotide microarrays, protein microarrays, single molecule real-time sequencing, ion semiconductor sequencing, pyrosequencing, sequencing by synthesis, sequencing by ligation, nanopore sequencing, chain termination sequencing, tunneling currents DNA sequencing, sequencing by hybridization, sequencing with mass spectrometry, microscopy-based DNA sequencing, RNA polymerase (RNAP) sequencing, and/or in vitro virus high throughput sequencing.

### Kits for Assaying

In some embodiments, a kit is contemplated for detecting at least one antibody directed against porcine morbillivirus (PoMV) in a sample. In embodiments, the kit comprises a PoMV polypeptide, the polypeptide including a fragment of at least 5, 10, 15, or 20 amino acids of a polypeptide having at least 90% identity to the amino acid sequence of SEQ ID NOs: 45, 48, and/or 49; and at least one reagent that allows the detection of an antigen-antibody complex between the PoMV polypeptide and the PoMV antibody present in a sample. In other embodiments, the PoMV polypeptide is labeled with a detectable marker. In some embodiments, the at least one reagent is labeled, or is able to be recognized by a labeled reagent. In another embodiment, the at least one reagent comprises a labeled antibody directed against pig immunoglobulin. In some embodiments, a reference sample devoid of antibodies is recognized by the PoMV polypeptide. In yet another embodiment, a reference sample containing a predetermined quantity of an antibody is recognized by the PoMV polypeptide.

In some embodiments, several types of kits can be envisioned and produced. One embodiment of the kit comprises the primers, reagents, and instructions for assaying PoMV expression using any of the described PCR methods. These kits would comprise: oligonucleotide primers and/or probes labeled with a reporter dye and quencher which specifically bind to PoMV to measure the expression of PoMV; PCR reagents; and instructions for use, either within the kit or available online. Other kits may analysis PoMV protein and comprise: one or more antibody(ies) to PoMV, which may have been conjugated to a reporter as described herein; an optional secondary antibody that binds to the PoMV specific antibody and is has been conjugated to a reporter as described herein; reagents appropriate for the kind of protein capture used, such as ELISA, EIA, or blotting reagents; and instructions for use. The protein kits may optionally further comprise of the substance which corresponds to the conjugated reporter. Alternatively, the kits can further comprise a substrate, such as a glass slide, a multiwell plate, or nitrocellulose paper, which the capture molecule, such as an oligonucleotide or antibody, may be bound to. Optionally, the kit can further comprise any other regent, such as, but not limited to, hybridizing buffer and label, for identification of PoMV in biological samples, using a specific probe. Further kits my assay PoMV in addition to one or more non-PoMV RNA, cDNA, and/or proteins, such as to, but not limited to, housekeeping genes, and would further comprise of capture molecules for the one or more non-PoMV molecules.

### Systems for Assaying

A “system” as used herein refers to a sample, a kit, and a device to detect the signal produced by the kit. The sample may be drawn blood, a tissue biopsy, lung lavage, sperm, the kit may be any of the kits contemplated herein, and the device may include any of the variety of available for the various assay methods described herein. For example, for detecting the bands resulting from semiquantitative PCR, an image may be taken using a camera, and the bands quantified using software such as, but not limited to, ImageJ (NIH, Bethesda, MD). ImageJ may also be used to quantify the bands created during blots, such as, but not limited to, Western blotting.

Florescent reporters can be read on systems such as, but not limited to, ABI PRISM 7700 Sequence Detection System or Lightcycler for PCR based assays, or on a Bio-Rad ChemiDoc or a Typhoon Biomolecule Imager for blots. ELISA and EIA can be read on devices such as, but not limited to, the Bio-Rad iMark Readers, BioTek Synergy Readers, or various BMG LABTECH star Readers.

### EXAMPLES

### Example 1

Using histopathology, metagenomic sequencing and RNA in situ hybridization, a novel morbillivirus in swine as the putative cause of an outbreak of reproductive disease characterized by fetal mummification, encephalitis, and placentitis in a commercial swine herd was identified. Based on the results in the Examples this novel paramyxovirus, found in the genus Morbillivirus, has been named porcine morbillivirus (PoMV).

### Materials and Methods

### Clinical Background and Samples

Twenty-two porcine fetuses from six litters (Litter A-F) originating from a commercial breeding herd in Mexico were submitted in the beginning of 2020 to the Iowa State University Veterinary Diagnostic Laboratory (ISU-VDL) for routine diagnostic investigation. The ISU-VDL is a National Animal Health Laboratory Network accredited laboratory that receives more than 85,000 cases per year, 75% of which are porcine origin. The breeding herd was composed of 2,000 sows and reported reproductive clinical signs characterized by an increased percentage (18% reported) of mummified fetuses and stillbirths.

### Pathology

Fetuses were selected by the herd veterinarian and were submitted intact for diagnostic investigation. All fetal necropsies and sample collection were under the direct supervision of an assigned diagnostic pathologist. At necropsy, the condition of each fetus or piglet (neonatal morality, mummified fetus, moderate autolysis or stillbirth) and the crown-to-rump (CRL) length was recorded in the case record. Heart, lung, and kidney were collected from all twenty-two fetuses submitted with the exception of one mummified fetus in Litter E with a CRL of 7 cm, were pooled by litter and fixed in 10% neutral buffered formalin. Brain, liver and spleen from stillbirths, spleen and liver from fetuses with moderate autolysis in Litter D, and placenta from Litter D and F were also fixed in 10% neutral buffered formalin. Fresh fetal thoracic tissue (lung and heart) and kidney were collected and pooled by litter for metagenomic sequencing, RT-qPCR and qPCR by litter. Fixed tissues were processed by standard technique and stained by hematoxylin and eosin (H&E) technique. Histologic evaluation was performed by BA. Sections from these same paraffin blocks were also used for RNAscope® ISH.

### Real-Time Quantitative PCR

All testing was performed at the ISU-VDL. Fetal thoracic tissue (heart and lung) or kidney (Leptospira PCR) homogenate were prepared, nucleic acids extracted, and PCR performed similar to what has been described previously. Briefly, PRRSV was detected using a commercially available NA and EU PRRSV-specific PCR assay (Applied Biosystems™ TaqMan® NA and EU PRRSV Reagents, Thermo Fisher Scientific) following the instructions. Primer and probe sequences for the detection of PCV2, PCV3, PPV1, and Leptospira are listed in Table 5. Internal control RNA (Xeno™, Life Technologies Corporation) was included in the master mix to monitor PCR amplification and detection of PCR inhibition. Two positive extraction controls, one negative extraction control, and a negative amplification control were included. RT-qPCR was performed (7500 Fast Real-Time PCR System, Applied Biosystems®, Foster City, CA) with the following cycling conditions: one cycle at 50° C. for 5 min, one cycle at 95° C. for 20 min, 40 cycles at 95° C. for 3 s, and 60° C. for 30 s. Samples with a Cq value <37 for either PRRSV genotype were considered positive. Samples with a Cq value <35 for PPV1, <35 for Leptospira spp, <37 for PCV2, and <37 for PCV3 were considered positive.

A real-time quantitative RT-PCR assay specific for PoMV was developed with the primers MBLV-900F and MBLV-988R and probe MBL-959P (Table 1). Primers and probe were synthesized by the Integrated DNA Technologies (Coralville, IA). The real-time PCR reaction was performed in a 25 μl PCR mixture that contained 12.5 μl 2×AgPath-ID RT-PCR Buffer (Applied Biosystems), 1 μl 25×RT PCR Enzyme Mix, 1 μl (0.4 μM) of each of the primers, 0.5 μl (0.2 μM) of probe, 4 μl nuclease-free water, and 5 μl extracted RNA. The amplification was performed at 48° C. for 10 minutes, 95° C. for 10 minutes, followed by 40 cycles at 95° C. for 15 s and 60° C. for 45 s. For each amplification plot a Cq value was calculated representing the cycle number at which the reporter signal was above threshold. Samples with a Cq value <40 were considered positive.

### Metagenomics and Bioinformatics Analysis

Total nucleic acid of fetal thoracic tissue samples was extracted and sequencing libraries were prepared as described previously. Sequencing libraries were sequenced on an Illumina MiSeq platform using a 600-Cycle v3 Reagent Kit (Illumina). Raw sequencing reads were pre-possessed and classified by Kraken v0.10.5-beta with Standard database. Unclassified reads were classified using Kaiju v1.6.2, and KronaTools-2.6 was used to generate the interactive html charts for the hierarchical classification results. Reads of the virus of interest, morbillivirus, were extracted from the classification results for de novo assembly using ABySS v1.3.9, iva v1.0.8, and Spades v3.11.1. Resulting contigs were manually refined, and curated and elongated by BLAST, SeqMan Pro and IGV visualization. The gap was closed by conventional RT-PCR with specifically designed primers.

Multiplex sequence alignments were generated by ClustalW. The phylogenetic trees based on whole genome sequence and amino acid sequence of the L protein (paramyxoviruses are currently classified based on the sequence comparison of L protein, the RNA-dependent RNA polymerases) were constructed from aligned sequences by the maximum likelihood model in MEGA version x (https://www.megasoftware.net/). The robustness of the phylogenetic tree was evaluated by bootstrapping using 500 replicates. Tree Of Life (iTOL, https://itol.embl.de/) was used for display, manipulation and annotation on the base of whole genome sequence and L protein amino acid sequence trees.

### Conventional RT-PCR for Genome Gap Closure and Whole Genome Sequencing

Conventional RT-PCR was used for genome gap closing and confirmation of the genome sequence assembled from NGS. One pair of primers was used for genome gap closing and 14 pairs were used for genome sequence confirmation (Table 1). The RT-PCR was performed in 25 μl mixtures containing 12.5 μl 2×PCR buffer, 0.5 μl SuperScript III RT/HiFi Platinum Taq Mix (Thermo Fisher Scientific), 1 μl (0.4 μM) of each of the primers, 7.5 μl RNA extract, and 2.5 μl DNase/RNase-free water. The cycling conditions were 60 min at 50° C., 2 min at 94° C., followed by 40 cycles of 30 s at 94° C., 30 s at 55° C. and 2 min at 68° C., and finally extension at 68° C. for 5 min. For the genome sequence confirmation, the RT-PCR reaction system and conditions were the same as those for genome gap closing with the exception of an annealing temperature of 48° C. instead of 55° C.

### Porcine Morbillivirus In Situ Hybridization

RNA ISH was performed using the RNAscope® 2.5 HD Reagent Kit—RED (catalog no. 322350 Advanced Cell Diagnostics, Newark, CA) according to the manufacturer's instructions for formalin-fixed paraffin-embedded samples (document numbers: 322452 and 322360 Advanced Cell Diagnostics, Newark, CA). Paraffin blocks stored at room temperature (RT) from each litter were retrieved and 4 μm sections were trimmed and mounted on Superfrost® Plus slides (catalog no. 48311-703 VWR, Radnor, PA). Slides were then dried overnight at RT and deparaffinized, treated with RNAscope® Hydrogen Peroxide, immersed in the prepared RNAscope® 1× Target Retrieval Reagent, rinsed, and treated with RNAscope® Protease Plus as previously described. Preheated probes were then dispensed on to the samples which were then hybridized for 2 h at 40° C. in the HybEZ™ Oven. The RNAscope® probe targeting the RNA of the L (replicase) gene of PoMV (catalog no. 858711) along with the RNAscope® positive control probe Ss-PPIB (catalog no. 428591) and RNAscope® negative control probe DapB (catalog no. 310043) used were designed and synthesized by Advanced Cell Diagnostics. After probe hybridization, six rounds of amplification were performed (RNAscope® 2.5 AMP 1-6) and slides were processed as previously described. Slides were visualized by a diagnostic pathologist using an Olympus BX43 bright-field microscope (Olympus Corporation, Tokyo, Japan).

### Results

### Gross Pathology and Pathogen Detection in Fetal Tissue by Real-Time Quantitative PCR

Twenty-two porcine fetuses from six litters (Litter A-F) with a crown-to-rump (CRL) length from 7 to 29.5 cm were submitted for diagnostic investigation. These included one neonatal mortality (aerated lung at necropsy), three stillbirths (full-term, fresh-type fetuses but non-aerated lung), fourteen mummified fetuses (in utero death occurred with sufficient time for complete dehydration of tissue), and four fetuses with moderate autolysis (in utero death occurred without sufficient time for complete dehydration of tissue). Gross evaluation of stillbirths and the single neonatal mortality was diagnostically unremarkable. Litter ID, sow parity, CRL by individual fetus and piglet that were submitted for diagnostic investigation, total born and number of affected fetuses in the litter as reported by the site are presented in Table 2.

To address differential diagnoses, known swine viral and bacterial reproductive pathogens, qPCR was used. PCV2, PCV3, PRRSV, PPV1, and Leptospira sp. were not detected by qPCR in any litter.

TABLE 2
Litter Information and Gross Pathology

| Litter | Sow | Crown to rump length (cm) and condition | Total | Mummified | Stillbirths |
|---|---|---|---|---|---|
| ID | Parity | of fetuses submitted | born | fetuses in litter | in litter |
| A | 6 | 24 (S) | 13 | 0 | 4 |
| B | 4 | 24 (N) | 12 | 0 | 4 |
| C | 6 | 29.5 (S) | NA | NA | NA |
| D | 2 | 7 (M), 9 (M), 14 (M), 15 (M), 28 (Mod), 26 (Mod) | 9 | 6 | 0 |
| E | 1 | 7 (M), 7 (M), 9 (M), 12 (M), 14 (M), 14 (M), 15 | NA | NA | NA |
| | | (M), 15 (M), 15 (M), 26 (Mod) | | | |
| F | 1 | 16 (M), 23 (Mod), 19 (S) | 9 | 3 | 0 |

S: Stillborn fetus;
N: Neonatal mortality;
M: Mummified fetus;
Mod: Fetus with moderate autolysis;
NA: Not available

### Metagenomic Sequencing

Two pooled fetal samples (fetal thoracic tissue) AB (encephalitis noted histologically) and DE (leukocytes in the epicardium noted histologically) underwent next-generation sequencing (NGS) using an Illumina MiSeq platform. After using an in-house bioinformatics analysis pipeline (https://pubmed.ncbi.nlm.nih.gov/29801460/), 693 and 118,772 paramyxovirus-like reads were detected and identified from the pooled sample AB and DE, respectively. De novo assembly obtained two contigs with 4,869 and 10,456 nucleotides from pooled sample AB and another two contigs from pooled sample DE. The nucleotide sequences of the contigs from the two different pooled samples were 100% identical but the contigs assembled from pooled sample DE were slightly longer with 5,042 and 10,705 nucleotides. Sequence analysis of these four contigs suggested the presence of a previously undescribed paramyxovirus of genus Morbillivirus with the two shortest contigs having fewer than 40% nt identity to PDV (accession #: KC802221) and CDV (accession #: AF014953) (3′ end) and the two longer contigs having more than 60% nt identity to these two known paramyxoviruses (5′ end). This paramyxovirus to be named porcine morbillivirus (PoMV).

### Genome Sequence Characterization

A complete genome sequence of PoMV (GenBank accession number: MT511667) was obtained by filling the gap between the two contigs using RT-PCR. Fourteen pairs of primers were designed according to the obtained genome sequence and RT-PCR products were re-sequenced, confirming the accuracy of the whole genome sequence. The genome size of PoMV was determined to be 15,714 bases and has a G+C content of 45.19%. The 3′ leader sequence of the PoMV was determined to be 55 nt with 13 out of the first 20 nt being highly conserved among morbilliviruses (FIG. 4). PoMV has a 5′ trailer sequence of 41 nt similar to other morbilliviruses that have a trailer sequence of 40 or 41 nt (FIG. 4) with the exception of feline morbillivirus whose trailer sequence is unusually long with 400 nt. The last 11 nt of 5′ trailer sequences are conserved in all morbilliviruses.

The genome of PoMV contains six genes (3′-N-P/V/C-M-F-H-L-5′) which is similar to other morbilliviruses. The pairwise alignment of the predicted gene and gene products in PoMV and other paramyxoviruses showed the highest nucleotide and amino acid identities with members of the genus Morbillivirus (Table 3). N, P, M, F, H, and L of PoMV have 56.3-65.8%, 44.3-61.4%, 62.4-67.3%, 50.2-62.5%, 31.7-52.7%, and 58.1-68.1% nt identities, respectively, and 56.3-69.0%, 26-48.3%, 60.9-77.6%, 42.2-64.2%, 15.7-45.4%, and 56.2-75.2% amino acid identities, respectively, with other members of the Morbillivirus genus and the highest identities to PDV and CDV and the lowest to FeMV.

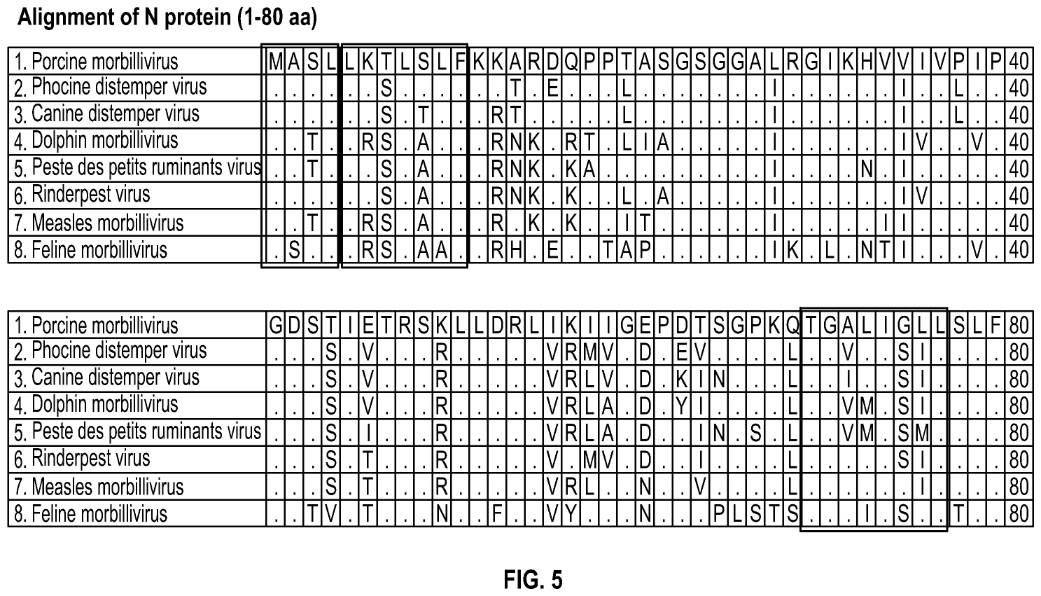

The conserved N-terminal motif MA(T/S)L in morbilliviruses containing the sequence MASL was present in the nucleoprotein (N) of PoMV (FIG. 5). A leucine-rich motif at the amino acid positions 4-11 and 70-77 are identified in the N protein of PoMV (FIG. 5). Two initiation codons were identified in the P/V/C gene of PoMV with the first one for the translation of P and V and the second one for translation of C. In addition, a UC-rich editing site (ttaaaagggg) was also identified in the P/V/C gene of PoMV. A conserved cleavage site (RRQKRF) was detected approximately 114 amino acid residues from the N terminus of the F protein. The F protein of PoMV also contains 9 out of 10 Cys residues and three potential N-glycosylation sites.

TABLE 3
Pairwise identities of predicted gene and gene products of
porcine morbillivirus compared with other paramyxoviruses

| | N | | P | | M | | F | | A (H) | | L | |
|---|---|---|---|---|---|---|---|---|---|---|---|---|
| Paramyxovirus | CDS* | AA^{#} | CDS | AA | CDS | AA | CDS | AA | CDS | AA | CDS | AA |

Morbillivirus (Orthoparamyxovirinae)

| | | | | | | | | | | | | |
|---|---|---|---|---|---|---|---|---|---|---|---|---|
| CDV | 65.8 | 69.0 | 61.4 | 48.3 | 67.3 | 77.6 | 62.5 | 64.2 | 52.7 | 45.4 | 68.1 | 75.2 |
| PDV | 66.6 | 70.3 | 61.3 | 50.1 | 68.0 | 78.8 | 62.4 | 62.6 | 51.5 | 44.6 | 67.5 | 75.8 |
| DMV | 62.7 | 66.4 | 57.2 | 43.9 | 68.4 | 77.9 | 64.1 | 65.5 | 48.2 | 39.9 | 65.3 | 72.0 |
| PPRV | 60.6 | 64.9 | 54.9 | 41.0 | 65.6 | 72.5 | 62.8 | 62.9 | 45.6 | 34.5 | 64.5 | 69.7 |
| RPV | 62.8 | 66.0 | 54.5 | 39.7 | 65.2 | 73.1 | 61.9 | 63.1 | 44.8 | 32.6 | 65.3 | 70.6 |
| MeV | 63.1 | 65.4 | 54.2 | 37.9 | 65.7 | 74.6 | 60.9 | 63.9 | 45.3 | 34.5 | 65.3 | 70.2 |
| FeMV | 56.3 | 56.3 | 44.3 | 26 | 62.4 | 60.9 | 50.2 | 42.2 | 31.7 | 15.7 | 58.1 | 56.2 |

Salemvirus (Orthoparamyxovirinae)

| | | | | | | | | | | | | |
|---|---|---|---|---|---|---|---|---|---|---|---|---|
| Salem virus | 50.7 | 45.0 | 33.5 | 21.2 | 53.8 | 47.9 | 45.9 | 35.5 | 27.1 | 11.9 | 52.6 | 46.8 |

Narmovirus (Orthoparamyxovirinae)

| | | | | | | | | | | | | |
|---|---|---|---|---|---|---|---|---|---|---|---|---|
| TupPV | 44.0 | 32.3 | 37.3 | 19.6 | 51.6 | 43.5 | 46.0 | 33.3 | 27.1 | 10.9 | 54.2 | 49.5 |
| MosPV | 43.7 | 36.0 | 36.7 | 20.9 | 54.1 | 48.8 | 46.0 | 35.9 | 30.1 | 10.9 | 54.4 | 49.6 |

Jeilongvirus (Orthoparamyxovirinae)

| | | | | | | | | | | | | |
|---|---|---|---|---|---|---|---|---|---|---|---|---|
| Tailam virus | 45.4 | 35.4 | 38.9 | 18.4 | 53.9 | 46.8 | 43.5 | 33.8 | 30.3 | 12.6 | 52.8 | 48.1 |
| MmlPV | 46.5 | 34.0 | 39.0 | 21.5 | 52.5 | 46.4 | 44.3 | 32.1 | 31.2 | 11.9 | 53.2 | 47.8 |

Henipavirus (Orthoparamyxovirinae)

| | | | | | | | | | | | | |
|---|---|---|---|---|---|---|---|---|---|---|---|---|
| Nipah virus | 42.1 | 30.3 | 34.4 | 18.8 | 52.7 | 45.2 | 45.3 | 32.1 | 30.8 | 11.7 | 51.2 | 46.2 |
| Bat | 42.9 | 33.1 | 34.6 | 16.9 | 52.8 | 43.3 | 46.4 | 32.6 | 26.8 | 9.4 | 51.1 | 45.0 |
| Paramyxovirus | | | | | | | | | | | | |

Ferlavirus (Orthoparamyxovirinae)

| | | | | | | | | | | | | |
|---|---|---|---|---|---|---|---|---|---|---|---|---|
| FdlPV | 41.7 | 25.7 | 35.1 | 13.3 | 46.1 | 34.9 | 42.9 | 29.0 | 28.1 | 10.9 | 48.2 | 39.6 |

Aquaparramyxovirus (Orthoparamyxovirinae)

| | | | | | | | | | | | | |
|---|---|---|---|---|---|---|---|---|---|---|---|---|
| AsaPV | 38.8 | 23.0 | 30.4 | 11.4 | 45.2 | 36.7 | 43.2 | 30.4 | 29.1 | 9.9 | 47.9 | 39.0 |

Respirovirus (Orthoparamyxovirinae)

| | | | | | | | | | | | | |
|---|---|---|---|---|---|---|---|---|---|---|---|---|
| BpiPV-3 | 37.1 | 20.2 | 31.5 | 12.8 | 45.0 | 35.7 | 40.9 | 25.8 | 30.4 | 11.7 | 48.2 | 37.3 |
| HPIV-1 | 38.1 | 20.4 | 31.1 | 11.2 | 45.2 | 37.1 | 42.9 | 26.8 | 29.8 | 9.7 | 47.9 | 38.7 |

Pararubulavirus (Rubulavirinae)

| | | | | | | | | | | | | |
|---|---|---|---|---|---|---|---|---|---|---|---|---|
| Tioman virus | 38.5 | 24.7 | 33.6 | 15.3 | 38.3 | 23.3 | 38.5 | 23.5 | 30.3 | 9.6 | 40.9 | 29.4 |

Orthorubulavirus (Rubulavirinae)

| | | | | | | | | | | | | |
|---|---|---|---|---|---|---|---|---|---|---|---|---|
| SipPV | 37.5 | 23.7 | 34.5 | 11.9 | 37.7 | 18 | 38.1 | 24.1 | 30.2 | 10.1 | 41.1 | 29.3 |
| PrPV | 38.0 | 23.8 | 32.8 | 12.8 | 37.6 | 18.6 | 39.2 | 28.6 | 29.9 | 10.1 | 40.8 | 28.5 |

Orthoavulavirus (Avulvirinae)

| | | | | | | | | | | | | |
|---|---|---|---|---|---|---|---|---|---|---|---|---|
| NDV | 37.0 | 23.1 | 33.8 | 13.2 | 34.4 | 20.8 | 40.5 | 25.2 | 28.7 | 11.5 | 40.2 | 27.1 |
| ApPV | 37.3 | 24.5 | 33.2 | | 33.7 | 19.6 | 41.7 | 26.2 | 28.2 | 11.2 | 39.9 | 27.2 |

Paraavulavirus (Avulvirinae)

| | | | | | | | | | | | | |
|---|---|---|---|---|---|---|---|---|---|---|---|---|
| AviPV-3 | 38.4 | 23.7 | 33.2 | 17.1 | 35.5 | 17.4 | 38.3 | 21.7 | 29.3 | 13.4 | 39.5 | 26.4 |

Mataavulavirus (Avulvirinae)

| | | | | | | | | | | | | |
|---|---|---|---|---|---|---|---|---|---|---|---|---|
| AviPV-2 | 38.8 | 26.9 | 35.2 | 13.9 | 35.7 | 20.8 | 42.0 | 26.8 | 27.2 | 13.6 | 39.6 | 26.8 |

Synodovirus (Metaparamyxovirinae)

| | | | | | | | | | | |
|---|---|---|---|---|---|---|---|---|---|---|
| WtlPV | 34.9 | 18.2 | 39.2 | 23.7 | 40.8 | 27.1 | 27.1 | 10.6 | 44.0 | 34.3 |

Unassigned to a genus or subfamily

| | | | | | | | | | | |
|---|---|---|---|---|---|---|---|---|---|---|
| WtPV | 34.7 | 15.8 | 38.4 | 16.6 | 36.5 | 20.2 | | 8.2 | 41.8 | 26.3 |
| WhPV, | 35.7 | 15.1 | 35.1 | 17.4 | 30.0 | 8.9 | 28.6 | 11.2 | 40.4 | 25.1 |
| WpssPV, | 34.8 | 17.2 | 29.5 | | 35.7 | 19.0 | 27.3 | 8.7 | 42.7 | 26.7 |

*coding sequence;
^{#}amino acid sequence
Abbreviations: CDV, Canine distemper virus; PDV, Phocine distemper virus; DMV, Dolphin morbillivirus; PPRV, Peste des petits ruminants virus; RPV, Rinderpest virus; MeV, Measles virus; FeMV, Feline morbillivirus; TupPV, Tupaia paramyxovirus; MosPV, Mossman virus; MmlPV, Mount Mabu Lophuromys virus 1; FdIPV, Fer-de-lance virus; AsaPV, Atlantic salmon paramyxovirus; BpiPV-3, Bovine parainfluenza virus 3; HPIV-1, Human parainfluenza virus 1; SipPV, Simian parainfluenza virus; PrPV, Porcine rubulavirus; NDV, Newcastle disease virus (Avian avualavirus 1); APV, Antarctic penguin virus A; AviPV-2 and 3, Avian avualavirus 2 and 3; WtIPV, Wenling triplecross lizardfish paramyxovirus; WtPV, Wenling tonguesole paramyxovirus; WhPV, Wenling hoplichthys paramyxovirus; WpssPV, Wenzhou pacific spadenose shark paramyxovirus.

### Phylogenetic Analyses

The phylogenetic trees were constructed using the whole genome sequences (FIG. 1) and the predicted amino acid sequences of the L gene (the RNA-dependent RNA polymerase gene) (FIG. 2) of PoMV and other members of Paramyxoviridae. In both phylogenetic trees, PoMV clustered with other morbilliviruses, with high bootstrap supporting a distinct subgroup. Both phylogenetic analyses also confirmed the findings from the above results of pairwise alignment and demonstrated that PoMV was most closely related to CDV and PDV; closely related to dolphin morbillivirus, PPRV, MeV, and RPV; and most distantly related to FeMV in the genus Morbillivirus. Overall, the data further supported that the PoMV identified is a new member in the genus Morbillivirus, subfamily Orthoparamyxovirunae and family Paramyxoviridae.

### Histopathology and Porcine Morbillivirus In Situ Hybridization

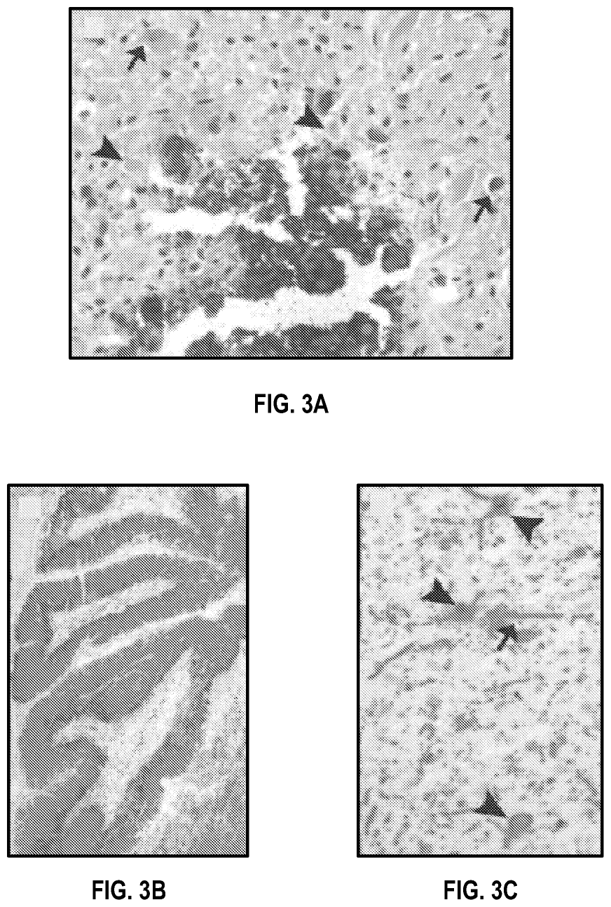

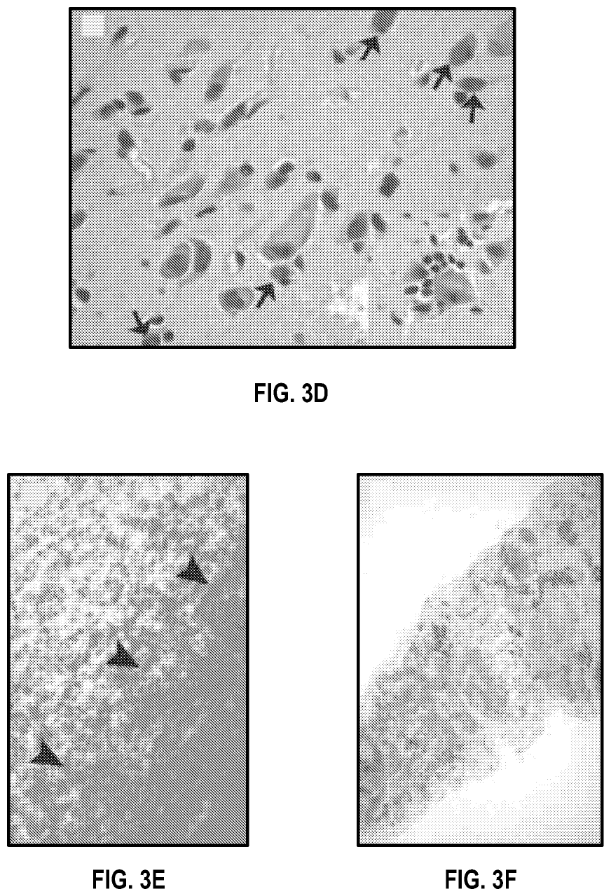

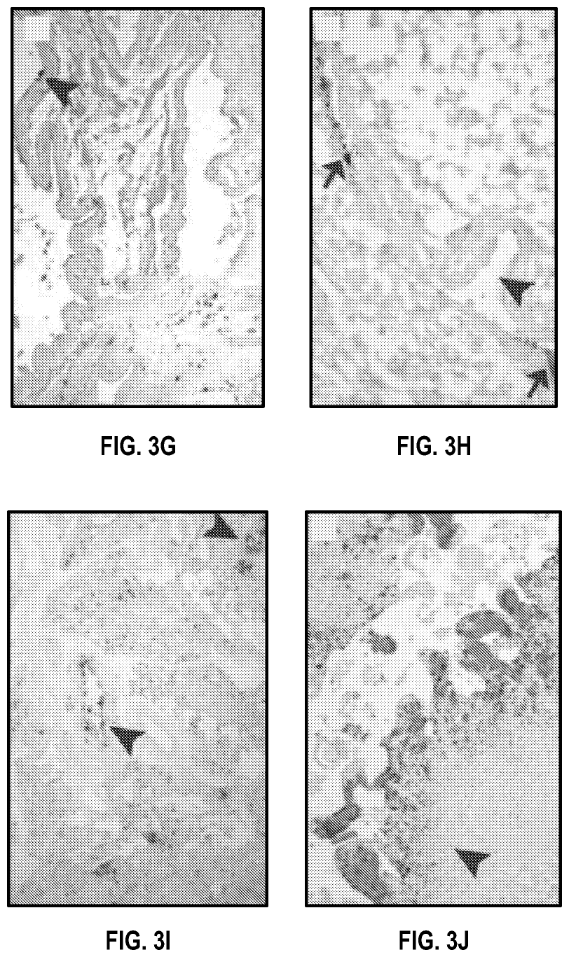

Histologic examination and RNA in situ hybridization (ISH) was performed by litter. A summary of histologic lesions and RNA ISH results are presented in Table 4. For all litters, the positive control probe, Ss-PPIB, was positive and the negative control probe, DapB, was negative on the single slide assayed (data not shown). The single stillborn fetus submitted from Litter A had multifocal areas of mineralization associated with neuronal necrosis and rarefaction in the cerebrum and brainstem (FIG. 3A). Cerebral vessels were occasionally surrounded by lymphocytes. Eosinophilic intranuclear and intracytoplasmic inclusions were observed in neurons (FIG. 3A) and glial cells in the cerebrum and internal granular layer of the cerebellum. Rarely, respiratory epithelium lining bronchi and bronchioles contained intranuclear inclusions. Histologic evaluation of the heart, spleen, and kidney was diagnostically unremarkable. Moderate autolysis of the liver precluded a thorough histologic evaluation. There was extensive PoMV RNA detected by ISH in the cerebellum (FIG. 3B; external granular layer, molecular layer, internal granular layer and white matter) and cerebrum (FIG. 3C; neurons and axons). PoMV RNA was also detected in clusters of respiratory epithelium lining bronchi and bronchioles, scattered lymphocytes and aggregates of lymphocytes in periarteriolar lymphoid sheaths in the spleen, and aggregates of tubular epithelium in rare tubules within the cortex of the kidney. PoMV RNA was not detected by ISH in the heart or liver.

The single full-term piglet submitted from Litter B had multifocal mineralization and rare satellitosis in the cerebrum (FIG. 3D). Adjacent to the lateral ventricle there was marked neuropil rarefaction, mineralization, neuronal necrosis, and leukocyte infiltration. Numerous viral inclusions were present in neurons (FIG. 3D) and glial cells. Histologic evaluation of the cerebellum, lung, heart, spleen, liver, and kidney was diagnostically unremarkable. PoMV was detected by ISH in the grey (extensive labeling) and white (less but still abundant labeling) matter of the cerebrum (FIG. 3E), low to moderate numbers of respiratory epithelium in multifocal bronchi and bronchioles, and individual lymphocytes in the spleen. PoMV RNA was not detected in the heart, kidney, or liver.

Litter C was also represented by a single stillbirth in which histologic evaluation of the cerebrum, cerebellum, lung, heart, and kidney was diagnostically unremarkable. Moderate autolysis of the liver and spleen precluded a thorough histologic evaluation. However, PoMV was detected by ISH in the endothelial cells of a single vessel in the cerebrum, scattered cells within alveolar septa and numerous lymphocytes within the spleen (FIG. 3F). PoMV was not detected by ISH in the heart.

Litter D was represented by three mummified fetuses and two fetuses with moderate autolysis. In a single section of one heart from a fetus with moderate autolysis, mononuclear leukocytes were present in the epicardium. Lung and kidney of fetuses with moderate autolysis were unremarkable. Autolysis of the spleen and liver from these fetuses and lung, kidney and heart from mummified fetuses precluded histologic evaluation. Despite severe autolysis and mineralization, leukocytes were observed in the allantoic connective tissue of the placenta. PoMV was detected by ISH in the alveolar septa, bronchi and bronchioles of the lung of both fetuses with moderate autolysis as well as bronchi and bronchioles of a mummified fetus. PoMV RNA was also present in scattered lymphocytes in one fetus with moderate autolysis and abundant lymphocytes in the spleen of the other. Mononuclear leukocytes in the allantoic connective tissue and allantoic epithelium of the placenta also contained PoMV RNA (FIG. 3G) as did rare renal tubular epithelium and endothelium of a vessel adjacent to the renal pelvis in a mummified fetus (FIG. 3H). PoMV was not detected by ISH in the heart or liver of moderately autolyzed fetuses.

Litter E consisted of nine mummified fetuses and one fetus with moderate autolysis. In a single section of one heart, the epicardium contained multifocal mononuclear leukocyte aggregates. The lung and kidney were unremarkable in the fetus with moderate autolysis. Autolysis of the heart, lung and kidney of mummified fetuses precluded histologic evaluation. PoMV was detected by ISH extensively in the conducting airway epithelium and alveolar septa of the lung (FIG. 3I), renal tubules, and in rare leukocytes in the epicardium of the fetus with moderate autolysis. ISH was not performed on mummified fetal tissues.

Litter F was represented by a single mummified fetus, fetus with moderate autolysis, and stillborn fetus. Expanding the allantoic connective tissue of the placenta were abundant mononuclear leukocytes. Histologic evaluation of the cerebrum, cerebellum, spleen, and liver of the stillborn fetus was diagnostically unremarkable. Kidney and lung of the stillborn and moderately autolyzed fetuses was unremarkable. Heart was unremarkable from all fetuses. Autolysis of the mummified fetus precluded evaluation of the lung and kidney. PoMV was detected in the epithelium of conducting airways and alveolar septa of the mummified fetus and fetus with moderate autolysis. There was extensive labeling in the allantoic connective tissue and mononuclear leukocytes throughout the placenta (FIG. 3J). PoMV was not detected in the lung, heart, or spleen of the stillborn fetus or heart of the fetus with moderate autolysis.

TABLE 4
Summary of Histopathology and RNA ISH by Litter ID*

| | Cerebrum | | Cerebellum | | Lung | | Heart | | Spleen | | Kidney | | Liver | | Placenta | |
|---|---|---|---|---|---|---|---|---|---|---|---|---|---|---|---|---|
| Litter ID | Histo | ISH | Histo | ISH | Histo | ISH | Histo | ISH | Histo | ISH | Histo | ISH | Histo | ISH | Histo | ISH |
| A | N, M, I | +++ | I | +++ | I | ++ | U | Neg | U | + | U | + | Auto | Neg | NA | NA |
| B | N, M, I, S | +++ | U | ND | U | ++ | U | Neg | U | + | U | Neg | U | Neg | NA | NA |
| C | U | + | U | ND | U | + | U | Neg | Auto | +++ | U | ND | Auto | ND | NA | NA |
| D | NA | NA | NA | NA | U | ++ | L | Neg | Auto | +++ | U | + | Auto | Neg | L | + |
| E | NA | NA | NA | NA | U | +++ | L | + | NA | NA | U | + | NA | NA | NA | NA |
| F | U | ND | U | ND | U | ++ | U | Neg | U | Neg | U | ND | U | ND | L | +++ |

*Not all fetuses were evaluated by histopathology due to autolysis and/or ISH. See text for specifics. Histo: Histopathology; N: Necrosis; M: Mineralization; I: Inclusions; Auto: Autolysis; NA: Not available; S: Satellitosis; ND: Not done; L: Leukocytes; U: Unremarkable. +: Minimal labeling; ++: Moderate labeling; +++: Abundant labeling.

### Porcine Morbillivirus Real-Time Quantitative PCR.

The fetal samples from Litters A, B, D, and E were further subjected to real-time qPCR to determine the viral loads in fetal thoracic tissues. All were positive for PoMV with samples from Litters A and E having higher viral amount (Cq values were 19.7 and 19.4, respectively) and samples from Litters B and D having lower viral amount (Cq values were 23.4 and 20.2, respectively).

### Summary of Results

The porcine morbillivirus, PoMV, reported herein was determined to be a cause of fetal death, encephalitis, and placentitis through the synchronous use of three independent and complementary lines of evidence that included pathology, metagenomic sequencing and in situ hybridization. Although several paramyxoviruses have been found, the existence of morbillivirus in domesticated swine was previously unknown. Analyses of predicted nucleotide and amino acid sequences of six genes revealed that PoMV have the highest nucleotide (31.7-68.1%) and amino acid (26-75.2%) identities in N, P, M, F, H, and L genes with the members in the genus Morbillivirus. Phylogenetic analyses based on both whole genome sequence and amino acid sequence of the L gene further demonstrated that PoMV forms a distinct cluster in morbillivirus and is most closely related to PDV and CDV. Similar to other morbilliviruses, PoMV has a 3′ leader sequence of 55 nt in length with 13 out of the first 20 nt being highly conserved among morbilliviruses. PoMV also has a 5′ trailer sequence of 41 nt analogous to other morbilliviruses that have a trailer sequence of 40 or 41 nt, with the notable exception of FeMV which has an unusually long trailer sequence of 400 nt. Similar to several other morbilliviruses, the nucleoprotein (N) of PoMV has the conserved N-terminal motif MASL. Other common features include a leucine-rich nuclear export signal and a leucine-rich nuclear localization signal (NSL) present in the N protein, two initiation codons and a UC-rich editing site (ttaaaagggg) identified in the P/V/C gene, a conserved cleavage site (RRQKRF), and 9 out of highly conserved 10 Cys residues detected in the F protein of PoMV.

While experimental inoculation of CDV in domestic pigs resulted in infection and viral replication, no previous morbillivirus is known to have naturally infected swine. Other viruses within the family Paramyxoviridae infect swine include porcine rubulavirus (genus Orthorubulavirus, subfamily Rubulavirunae), Menangle virus (genus pararubulavirus, subfamily Rubulavirunae), Nipah virus (genus Henipavirus, subfamily Orthoparamyxovirinae) and porcine parainfluenza virus 1 (genus Respirovirus, subfamily Orthoparamyxovirinae). Among these, only porcine rubulavirus and Menangle virus are thought to cause fetal mummification and stillbirths as observed with PoMV. Histologic lesions of PoMV are similar to Menangle virus and include encephalitis, viral inclusions and nonsuppurative myocarditis.

Similar to PoMV, MeV has been reported to be transmitted vertically in humans resulting in premature stillbirth, stillbirth, premature birth, neonatal death, or congenital measles. Viral inclusions were observed in human congenital MeV infection, and MeV was detected in placenta and splenic lymphocytes by immunohistochemistry. In the porcine case presented here, viral inclusions were commonly observed in the cerebrum and cerebellum and rarely in the respiratory epithelium lining conducting airways, and PoMV was demonstrated in the placenta and splenic lymphocytes as well as the cerebrum, cerebellum, lung, and to a lesser extent in the kidney and heart by an in situ hybridization assay. Interestingly, the degree of viral involvement within the monochorionic placenta and impact of MeV on monozygotic twins was inconsistent, with one in utero death at 32 weeks gestation and one surviving infant with no clinical signs of MeV infection. This observation along with the placentation of swine, large litter size, and observations from other swine viral reproductive pathogens, likely accounts for the variable impact on litters, which was characterized by fetal and piglet death at variable stages of gestation resulting in fetal mummification, in utero death, and stillbirth as well as the variability of ISH staining observed among fetuses and between litters.

Cellular tropism of PoMV as determined by ISH aligns with other morbilliviruses. A common entry receptor for morbilliviruses is CD150 or signaling lymphocytic activation molecule (SLAM), which is expressed on activated lymphocytes, dendritic cell subsets, and macrophages with CD150-expressing cells also present in the alveolar lumen and lining the alveolar epithelium. PoMV RNA was observed in the alveolar septa, lymphocytes of the spleen, mononuclear leukocytes in the placenta and mononuclear leukocytes in the epicardium suggesting that PoMV may also utilize CD150. Morbilliviruses infect epithelia using nectin-4, which is expressed on the basolateral surface. PoMV RNA was detected in the allantoic epithelium of the placenta, epithelium of bronchi and bronchioles, and rarely renal tubular epithelium. Additionally, PoMV was detected in the endothelium of a cerebral and renal vessel with the most extensive staining in the cerebrum and cerebellum including neurons. Previous studies have shown that no detectable expression of SLAM was found in human neurons and extremely low expression of nectin-4 was detected in central nervous system cells and tissues (https://www.proteinatlas.org/ENSG00000143217-NECTIN4/tissue). In contrast, CD46 is a widely distributed complement regulatory protein expressed on all nucleated cells with labeling noted in the cerebral endothelium as well as ependymal cells lining the ventricles, choroid plexus, neurons, and oligodendrocytes. Accordingly, PoMV may also utilize CD46 as a cell receptor similar to some strains of MeV.

Having described an exemplary method of detecting PoMV in a sample, PoMV has been identified and characterized from the swine fetuses. PoMV may be used to create a variety of vaccines as disclosed herein. The full genome having been determined may be used as used either alone or in an expression vector to express the virus in a host cell. Additionally, as the nucleotides encoding the various proteins of PoMV has been identified, these nucleotides, proteins, fragments and/or variants thereof may likewise be used in the vaccine compositions as described herein. PoMV, fragments or variants thereof, may also be used to generate antibodies and hybridomas as described herein.

TABLE 5
Real-time Quantitative PCR Primers and Probes

| Name (SEQ ID NO.) | Sequence |
|---|---|
| PCV2 forward primer (SEQ ID | 5′-GACTGTWGAGACTAAAGGTGGAACTGTA-3′ |
| NO: 32) | |
| PCV2 reverse primer (SEQ ID | 5′-GCTTCTACACCTGGGACAGCA-3′ |
| NO: 33) | |
| PCV2 probe (SEQ ID NO: 34) | 5′-6-FAM/CCCGTTGGAATGGT/MGB-3′ |
| PCV3 forward primer (SEQ ID | 5′-TGTWCGGGCACACAGCCATA-3′ |
| NO: 35) | |
| PCV3 reverse primer (SEQ ID | 5′-TTTCCGCATAAGGGTCGTCTT-3′ |
| NO: 36) | |
| PCV3 probe (SEQ ID NO: 37) | 5′-VIC/ACCACAAACACTTGGCTC/MGB-3′ |
| PPVI forward primer (SEQ ID | 5′-CCAAAAATGCAAACCCCAATA-3′ |
| NO: 38) | |
| PPV1 reverse primer (SEQ ID | 5′-TCTGGCGGTGTTGGAGTTAAG-3′ |
| NO: 39) | |
| PPVI probe (SEQ ID NO: 40) | 5′-6-FAM/CTTGGAGCCGTGGAGCGAGCC/IA |
| | Black FQ-3′ |
| Leptospira spp. forward | 5′-AAGCATTACCGCTTGTGGTG-3′ |
| primer (SEQ ID NO: 41) | |
| Leptospira spp. reverse | 5′-GAACTCCCATTTCAGCGATT-3′ |
| primer (SEQ ID NO: 42) | |
| Leptospira spp. probe (SEQ | 5′-6-FAM/AAAGCCAGGACAAGCGCCG/ |
| ID NO: 43) | BHQ1-3′ |

### Example 2

| PoMV Genome Sequence | |
|---|---|
| SEQ ID NO: 44 | |
| 1 | acttacctta ggatccatga tctgatcgcc aagagtgggt gcagggatct gagacctagt |
| 61 | catggctagc ttactaaaga ctttatcact tttcaagaag gcacgtgatc agccccctac |
| 121 | agcatctggg tcaggaggag cattaagagg tatcaaacat gtggtaattg tcccaattcc |
| 181 | tggtgactct acgattgaga ctcgatcaaa gctcttggac cgactgatta agatcatcgg |
| 241 | ggagcccgat acaagtgggc caaagcaaac cggagcacta atcgggctgc tatcactttt |
| 301 | tgtggagtcc ccaggacaat tgatccaaag aatcgttgat gatcctgata tcagtatcaa |
| 361 | attggttgag gtggtcccaa gtgatgagtc aaaatcaggt ttgacgtttg cttctcgagg |
| 421 | tgccaatttg gatgctgagg ctgaccagta cttaggagaa gatgagtact caagcgagga |
| 481 | aggggctgta caaggttggt ttgaaaataa tgaaatcagg agtgttgagg tgggcgatgc |
| 541 | tgaatatttc aacatcatcc ttgcatcagt attggcccaa gtttggatac ttctggcgaa |
| 601 | ggccgtaacc gccccggata ctgctgcaga ctccgaaatg agacgctggg tcaagtatac |
| 661 | ccagcagagg cgggtcatcg gtgactttag aatgtcacgt ttatggttag actcagtcag |
| 721 | gaacagaatt gctgatgatc tgtctctccg gagattcatg gtggcattga tcttggacat |
| 781 | aaagagatct cctgggacga agcccagaat cgctgagatg atctgtgaca ttgacaacta |
| 841 | cattgtagaa gccgggctag caagctttat cctcacgatc aagtttggga tagaaactat |
| 901 | gtacccggca ctgggcttgc atgagtttgc tggtgagtta acaaccattg aatccctgat |
| 961 | gattctatac caaaagatgg gtgaaaccgc tccttacatg gtcatactag aaaattccat |
| 1021 | tcagaataaa ttcagcgcag ggtcataccc tctactttgg agctacgcca tgggggtagg |
| 1081 | tgttgagctg gagaattcca tgggggggct taattttggt agaacatatt ttgatcctgc |
| 1141 | gtatttcaga cttggacaag agatggtacg tagatctgct ggaaaggtca gcacctcttt |
| 1201 | tgctgctgag ttgggaatat cgaaagagga tgcgcagctg gtttcagaaa ttgccaccag |
| 1261 | agcaaccgat gaaagagttg ggaggtccac aggagtcaag caaacccaga tatcctttct |
| 1321 | gcacgctgaa aaaccgggga aggggattca atcagaggat gccattattg ctgaaggtcg |
| 1381 | cccaaagaaa accagaaaga agacatctga tcgtctcagt tccgatgaca tcgacgatga |
| 1441 | tgttgtggga ccaactacca gctttgatga tggtcgacgg acagatcagg accccgactt |
| 1501 | agacgaatcc tcctatgacg agaaatctgc tgaagctata gcaaagatgc gtctaatgac |
| 1561 | taaattgcta ggaaaaacct ccccagatac tcccacattc cgcacctata atgacagaga |
| 1621 | actactcaac tgaccaagca atgcccatca gcccattcta ccactgacca ctcccaactg |
| 1681 | cattataaaa aacttaggac ccaggtccac aaatcacaga tcacaaccac agataacacc |
| 1741 | agtcaccctc tccaatggct gaagagcagg cccagcatgt cagcaaaggg ctggagtgca |
| 1801 | ttaaagccat cagagataat cctctctgta ttgaggagat tcaggaaatc aagatcatcc |
| 1861 | gcgacaccgt cttcaacgaa cgtgaatcta ccgtcgactc tgatcaggga gaaggggatc |
| 1921 | tcgatgagga tctcgatgaa tcacagacgg caacaagtgg cccagcaaga aagggttcag |
| 1981 | tacaatcaga ccctcatgga actagatcaa tggatggacc acattttaag ggatcagatg |
| 2041 | gtgaaggagt tggtatcagc acaagatctg acatacagca ctactatgtt cacagccacg |
| 2101 | gcggtgaaga ggttacggga atccaggatg ctgacagtct catggtacca acagataaca |
| 2161 | accgtggtcc cgatgacgac gagggaacgg atagcgctga tgagggcatt gatgaatcta |
| 2221 | gtgaggatga tccctcaaaa cctactgccc atgacagggg atttgctccc actcctgctg |
| 2281 | cgactagatc agcagacgtg tcaaccgtga tggatgacga actaaccttg ctgttggggg |
| 2341 | ctcataccaa gaagaagagc agagaggacc cggggagaca gctgagtttt ccgaaacaac |
| 2401 | ctagaattga acccctcgag ccgagaccag aatctattaa aaagggcaca ggcgtgaaat |
| 2461 | taatctcatc tggaatggcg atcgactcca catcgatcaa tggtgcaacc ccatctgcgc |
| 2521 | tcgggtcacc agcgaaatca tcagagctca gtgtacctgc ggcgaatgtc cccaagtctg |
| 2581 | tcccgactgc aaaaatgacc ctgagatccg gacccgagtc tggtatgcat actcagtcgg |
| 2641 | agccagcccc ggtcaatgaa gaggaggaag agtacgaaga tgagcttctt gcagagatac |
| 2701 | tagagttgaa agatgctgtc accaaaatca atgaggataa tcaactgatt ctgtcgaaat |
| 2761 | tggatgctgt tctgagcctc aagggagaga ttgattccat cagaaaacaa ctcaacaaac |
| 2821 | aaaatattgc aatatcgact atcgaaggac atatttcaag cattatgatc gcaatacccg |
| 2881 | gattcggtaa acctgaccca gatgggcttg cagaatcaga cgtgaatcct gagctccgcc |
| 2941 | caataattgg tagagactca ggacgtgcat tggccgaagt tcttaagaaa cccccagtca |
| 3001 | ccaccgggaa aacagcgcaa ccctccatca agtcaggttc caagcgccag ctgttaaagg |
| 3061 | agatgaggct tcaacccatc aacccacgaa ctagctctgc aatcaagttc aacccggggg |
| 3121 | acgacgtccc ctctaagagc gttattagtt ccctgataaa gtcaagcagc ctggatcaga |
| 3181 | gccacaaggc caatatgtta actcttctca aggaaatcaa gggagacaaa aacgtcaagg |
| 3241 | aattccacca aatggttctg gagattatca gggcttaata gcaatgcttc ttcagcataa |
| 3301 | ccccccaata taactcgctc aagcataggt gtagcatgca ttataaaaaa cttaggatca |
| 3361 | aagttgatcc atcagtaact caaaaatgac agaagtgtac gactttgacc aatcgtgctg |
| 3421 | ggattcaaag gggtccctcg ccccaatctt cccgaatact tatccggatg gtcgattgat |
| 3481 | acctcaggtt cgggtcatag accctgggct tggcgacaga aaggaagaat gtcacatgta |
| 3541 | tcttttcttg ttggggataa ttgaggacag tcaaaatgag ggcccatcaa cagggcgggc |
| 3601 | attcggatcc ctcccgttag gtgttggtag atctatggca aagccggaag aactactcaa |
| 3661 | ggaggcgata accctggata ttgtagttcg gaggacagca ggagtgaatg agaagctagt |
| 3721 | tttttataac aacacccctt tgaatgttct cctgccatgg aaaaaagtct tgaccaatgg |
| 3781 | aagcatcttt aacgcgaacc aggtctgcgg cggagtgaac cttataccgc tggatgtgtc |
| 3841 | acagagattc agagtggtgt atatgagcct tactcgtctg tcagatgatg ggtgttatcg |
| 3901 | agtgcctagg gggatgctag attttagatc tgggaatgcg attgctttca atatactagt |
| 3961 | aacgattcaa gtcaacactg ttcagacaag cgggggtgat tatatcagtg caccaagagg |
| 4021 | gaccatagcc accttcatgg tccatattgg gaactttaaa cggaagacaa ataagcagta |
| 4081 | ttcagctgat tactgcaaga tgaagataga aaaaatggga ctagtatttg ctcttggtgg |
| 4141 | aatcggaggt accagtctcc atgtgaggtg cacagggaag atgagtaaag ctcttaatgc |
| 4201 | ccaattaggc ttgaaaaaga ttctgtgtta tccgttgatg gaagttaatg aggatctcaa |
| 4261 | tagacagctc tggagagctg agtgcagcat cacccgtgtc caggcagtct tgcaaccatc |
| 4321 | tgttccgaat gactttcgta tatacaatga tgttatcatc aacgataacc aggggctgtt |
| 4381 | caagatccta taagagggat agaatatgtg cattttcaac agcatgccca ggccttgcac |
| 4441 | ccgcggcaca caactcagcc ctctcccccg gtcgagggcc aagcagtgac cggcatgtgc |
| 4501 | acccaaggtc aaaagcaccc ctcaggacca tcacccccga tacagactcc acaagtgccc |
| 4561 | atcacaacac agtacactca caagcacaca tcctccccac agggatccaa aagcacccca |
| 4621 | ctccccaaat cggcaagccg accccatctc acatctcaac ctcaaatcac acacccccag |
| 4681 | gacgtagcac tatctgaggg aaccgcacaa tgcccattct acactcaccg ccatagagcc |
| 4741 | tccaactagc tcgctcccgc actttctcta cttagtcgat agattgagca ttaagaaaaa |
| 4801 | ctgaggaccc aagtcccaca tcagcccgga ctcccaccgt ccaccagggc ccacggccag |
| 4861 | accaacgagc agaccaacgg aagcaccacc agaacccgag agggagcagc gcaggtccaa |
| 4921 | ggcagggcca ggtcaagcgc tgcacccccc cgggcagacc ccccccatcc ctccccgaga |
| 4981 | acccagaccg cacagactga acaggtccgc atatccaatc gcgcaacccc gagacaaccc |
| 5041 | gggagccctc acgaccgacc ttccgtccgt ccgacccacc aaatcctgtg ccccaggaca |
| 5101 | gctcgccaac aaaccctcca ttagaaaccc gaccaccacc ccactcccct ccccggcgga |
| 5161 | ccgaaccaag agaagccctc tcaatctccc ctgaaggtgc caggctgcct tccccctcca |
| 5221 | cctcgccatg gtcaaaccga aaacagccct ttgcccaccc acaggcataa tccttgaagc |
| 5281 | tctcttggga ttcatactag tggaattatc gtatagtcaa gtacactggg gtaatctgtc |
| 5341 | taagatagga gtcctgagca ctgaaagcat gacatacaaa attatggcac ccggggcgag |
| 5401 | ccagtattta gttataaaac ttatgcctaa tgtgaccggc ttagacaatt gtactagcag |
| 5461 | tgaagttgct gagtacgaat ctttactggt gaacatcctt ggccccatca ataaagcttt |
| 5521 | gcaggtaatg acaaccagca tgaggcctgc ccagactaca tacgggtcac gtcgacagaa |
| 5581 | aaggtttgcc ggtgttgtcc ttgcaggggc agcattagga gttgccacag cagctcagat |
| 5641 | aacggctggg gtagcattac accagtcaaa tatgaatgcc caggctattc gggatcttaa |
| 5701 | aaatagcctg gagaaatcca atcaagcaat tgaagaaatt aggcaggcta cacaggaagt |
| 5761 | agtcgtagcc gtacaggggg tgcaggatta tataaatcat gaacttattc caacaatcag |
| 5821 | ccaaatgtcc tgcgagatca taggacagaa attagggcta aagctgctta ggtactatac |
| 5881 | tgagttgctg tctatattcg gccctagcct gagggaccct ctctctgctg agataacaat |
| 5941 | ccaggcattg acttatgcct tgggtggtga tatatataag gtgattgaaa aactagggta |
| 6001 | ctccggtcat gatctagcag ccgttctcga aagtggggga ataaaaacac gagtggttca |
| 6061 | cgctgatgta caatcaaaat tcataatctt gagtatttca tacccaacat tatccgaggt |
| 6121 | caagggggtc gttgtacata agttagatgt cgtttcatac aacattggct cccaagagtg |
| 6181 | gtacaccgca gtccccaaat ttgtcgccac gaatggttac cttatctcta acttcgatga |
| 6241 | atcgggctgt gctttagtta ccggggtatc aatttgcagc cagaatgcgt tgtatcctat |
| 6301 | gagttctgta atgcagcagt gtatgagggg agaaactgga tcttgtgcta gaacattagt |
| 6361 | ctctggaaca attgggaata gattcatact ttcaaaaggg aatattgtcg cgaactgtgc |
| 6421 | atcaattatg tgcaaatgca caacttctgg aactatcatc aatcaagatc cggacaaatt |
| 6481 | acttacattc atcgcgactg atctatgccc acttgttgag attgagggaa taacaatcca |
| 6541 | ggtcggtact agagagtacc cagacactgt ttatgaacat aggatcaacc ttggccctgt |
| 6601 | aatcccactt gagaaactcg atgtcggtac aaatcttggt aatgcaatct ccaagttaga |
| 6661 | agatgcaaag gatcttctcg atgcttctaa ccaaatcctt gatcttattc aagattctag |
| 6721 | aatgtccatt ggcaaatata taatttgggc tgctgtagga ttttctatct ttggcgggtt |
| 6781 | gttattactg tgctgctgta agaggtacta ccagaaagtc tcagcaaatt ctactgtcat |
| 6841 | taatcctacg gtcaaaccag acttgacagg gacctcaaga tcttacgtca agtcattgtg |
| 6901 | aaccctggtg cctgtttggc tttgtaaagg gattagaggc agcctagcct ttgcgtgatt |
| 6961 | ggtaacccct gcactcgtcc cttggatttt gtaaaacttt tacaccttct caatagccaa |
| 7021 | ctattaagaa aaacttaggc tccaagtcct acagccatgc cccctcttgg agacaaaagc |
| 7081 | aatgctttct acaaggattc tgccagagct aatctcccta tccttaacat caatggattt |
| 7141 | ctacctgccc atcaccgagg tcccctcctc ttgctactgg caactgtctc actagtgggt |
| 7201 | ttggttgcat tgattgcaat tgcaggggtg agattccaca agtctaccac tatggcaaag |
| 7261 | gagactaact cgctgatcca ggaaaacatc catcacactg acctactaca ccaccaggtg |
| 7321 | atggatgtgt taacccctct tttcaagata atcggggaag aggttggcct tagattacca |
| 7381 | cagcgattag cagagatcaa gcaatttatc ttaacgaaga cgtcattctt caatccagat |
| 7441 | agagaatttg atttcagaga cctccattgg tgcattaatc ctcctgaccg agttaaggtc |
| 7501 | aatttcagta aattttgcca ggccattcac atcgacaatg ggatacgact cttaggtggt |
| 7561 | atattcattg accagttctt atcacatagc aaatcagata tattccctgg tagggtatgc |
| 7621 | ccaaatgggg ttacaactag gggagagatt atatcacctg ccttatctct tactagtcta |
| 7681 | agtgcaaaac atcttgggtt agtcgataac attatgtttg ctatggcgga tggtgtctat |
| 7741 | gctaagactt atgtactttc ccacttttcg gagggagggg tcaccgaaac gggagagatt |
| 7801 | agggtttttg agatagggtt gattaggggt tggctgggtg acctcccagt gcttcatctc |
| 7861 | acaaacttta tccatgcaaa tgtagattca ataggcagac actgcacact agctgttggg |
| 7921 | gaactgaaac tcgcatctat ttgtacccac gcgcgggcaa taagagtggt gaggaatgtg |
| 7981 | gatagtcacc caacttctgt ctcggcaatc tctatgggtg tattcggatc agactactac |
| 8041 | caacaaagtg ttgaagtgat cccagtgtat gatgaatctg tacagcaaat tcatctgtcg |
| 8101 | aaccaccgag ggtttataaa ggactcaaag gcatactggg cgttccccgc tgaagctgac |
| 8161 | actaattcca ctcgatggtc caactgcttg acgaaagtct gcacctctcg taacttaccc |
| 8221 | ttttgcaacc tgaccagctg ggagccattc aatactacat atcccgcagt gtatgccata |
| 8281 | ttggtccttg aggttagtgt tcacagtgat atctcgatgt tcatagacga gtccctaggt |
| 8341 | ccattaatct tgaatgggta tggtatggat ttatactcga acagccatga ggaccagcat |
| 8401 | tggttgacta tcccaccaaa aagtggcaat gttctgggtg ttatcaacaa gttagtggtc |
| 8461 | aatgggagtg cggcggtgct cccgtacata cttagctatg ctgcatcagc tggccatgaa |
| 8521 | ccatgttatg cccctgtcca gcactcttta cccagtgaca ccaacacgct tgctgaatca |
| 8581 | aatatagttg tcctgtcaac cggaaagttc agatacgttt ctgcaactta tgacacatcg |
| 8641 | aggagcagcc atgcaatagt gtattatatc tacaatccta cagagggaac atcctatttt |
| 8701 | ttccccttcc gacttacaac aaggggaatt ccagcattcc tgcgcataga gtgctttgta |
| 8761 | tgggttgaga aaatatggtg catacatgtg taccggtacc attctaccgt cagtaaggta |
| 8821 | gaaccagttg tggaaactat ggtcagattg gaatttaagt gtacctcctc ttaagatccc |
| 8881 | tccgttctcc tttgaccctt aaactggaat ctagaagttt aagtaccacc tgctacatgc |
| 8941 | ctatccagca ctccataact cttctgagag accataaaaa aacttagggt ccaggtaagt |
| 9001 | ttgcactatg gactctatct cagttaacca gattctctac ccggaagttc atctcgacag |
| 9061 | ccccatcgtg accaataagt tagtagctat cctagagtat gccaatctaa atcacaacta |
| 9121 | ccagttgata gacccgaccc ttattgagaa tattaaacag cggatcaatg aaggatactc |
| 9181 | caaccaaatg atcatcaact atatcaacgt cggacacatc ataaatgatc tactggctac |
| 9241 | tttgcctaaa acaactcata ttctgtatcc taattgtaat aaagatctgt ttagggcaga |
| 9301 | agatgcgttg gttactcaaa aattgctaga tttgttcaag aaaggaaaca cgatatatac |
| 9361 | gaaaatatcc ggagggataa ttagatgctt aaccaacctt aatcaatcac tgggtcttgg |
| 9421 | aggcaatctc cctgaaatct accataaccc aatacaagat ttaggagtta tgatgcaaag |
| 9481 | ttcaaagtgg ttcggttcat tcctgttttg gttcaccatc aaaaccgaga tgaggtctgt |
| 9541 | aataaaatca agtactcaca actgtcaaaa aaggagatat atcccgtcat tcaccaccgg |
| 9601 | aaagaatttc aaaatccttg tttcgagaga cctcgtttgc cttgtgaacc ttgctgacaa |
| 9661 | gagggtttac tacttaacat ttgaaatggt cctgatgtac tgtgatgtca tcgaaggccg |
| 9721 | attaatgact gacacagcca tgtgtattga ttcaaaattt gctcagctac gtaaaagggt |
| 9781 | ccactatctc tgggatctga ttgacggatt ttttcctgac ttagggaact ctacctacca |
| 9841 | gctcgtagcg ttgcttgaac cactttccct cgcttacctc cagctgaaag atgcaacaga |
| 9901 | tcagctcaaa ggcgcctttc tgtctcattg ctttacagag attaaagaga ttttagataa |
| 9961 | ccaaggtttt actttgccag acacatatgc tcaattaatc gaggctttag atactatctt |
| 10021 | tatcactgat gatatccatc tgacaggtga gattttttct ttttttagaa gttttggcca |
| 10081 | tcctcggtta gaggcagtga ctgcagcaga gaatgtgagg aagcacatga atcagccaaa |
| 10141 | agtcatctcc tatgaaaccc taatgaaagg tcatgcaatt ttctgCggga taatcattag |
| 10201 | cggattccgg gaccggcatg gagggagttg gccccctttg attctccctg agcatgcttc |
| 10261 | accagtagtt aaaaacgccc aggcttcagg tgaagggcta acatacagcc agtgtgttga |
| 10321 | caattggaaa tcattcgtgg gtatgaggtt taagtgtttt atgcccttga gccttgacag |
| 10381 | tgacctaaca atgtatctaa aagataaggc tttatctgcc ctcaggaaag agtgggactc |
| 10441 | tgtgtatccg aaagaatatc tacggtacaa cccccccaag tcctcgggat ctagaagatt |
| 10501 | agttaatgtt ttcctagagg attccgaatt cgacccttat aatatgatca tgtatgtaat |
| 10561 | atctggagaa tacctgagag acccggactt caacttgtcc tatagtctca aggagaaaga |
| 10621 | aatcaaagaa gtcgggaggc tattcgcaaa aatgacatac aagatgagag catgccaggt |
| 10681 | gattgctgaa aacttaatcg ccaatggtat aggaaagtat ttcaaggata acggcatggc |
| 10741 | aaaagacgaa catgacctaa caaaagcatt acataccctg gctgtctctg gggtccccaa |
| 10801 | agataaatcc caaaagcacc gagaccccag aactaaaata gataaccact ataatagaga |
| 10861 | aacagggggt ctcccaaaat gtcaacttgg aggaaaacag ggcccatcag gacatcggac |
| 10921 | gggtttcatc agtggccggg ctgatgataa tgacatagac ctttatgaga ctgtaagtgc |
| 10981 | ttttataacc actgatctca aaaagtattg tttaaattgg cgttatgaga caataagcat |
| 11041 | ctatgcccag aggttaaatg agatttatgg actcccttct ttctttgaat ggttacataa |
| 11101 | aaggttagag aagtctgtac tgtatgtcag cgatcctcat tgcccaccag atctcgacca |
| 11161 | ccatatcaac ctggatgagg tcccaaatca acaaatcttt atcaaatacc cgatgggtgg |
| 11221 | aattgagggg tattgccaaa agttatggac cataagcact atcccctatc tttacttgtc |
| 11281 | agctcatgaa tgcggagtga gaatagcttc tttagttcag ggagacaacc agactatagc |
| 11341 | tgttaccaag cgagttccga gttcatggcc ataccacctt aagaaggcag aagcagctag |
| 11401 | agtcacaatt aattatttca tgattttacg ggaaagactt catgatatag gtcaccattt |
| 11461 | aaaagctaat gaaaccataa tttcatccca tttttttgta tattcaaaag ggatttacta |
| 11521 | cgatggcatg ctaatctcac agtcccttaa aagcgtagcc cgatgtgttt tctggtcaga |
| 11581 | aactattgta gatgagacta gagcagcttg tagcaacata tccacaaccc tggctaaatc |
| 11641 | aattgaaaaa ggatttgata gatatctagc ctatgccctt aaccttatca aaaccataca |
| 11701 | gcagctgttg atttcactgg gctttaccat caatggatcc atgacagacg atgtggttga |
| 11761 | acccctttta aaagatcata gaattataac aaagatggca ctcctccctg ccccgatagg |
| 11821 | aggattcaat tatttgaaca tgagccgcct gttcgttaga aatataggcg atccggtcac |
| 11881 | atcatcattg gctgatttaa aaagaatgat taaagcaaat ctgctgagtg aaacaattct |
| 11941 | tcatcaagtg atgactcaag ccccaggtga ttcgtcattt ttagattggg caagtgatcc |
| 12001 | gtactcagct aatctcccat gcactcaaag catgaccaga gtccttaaaa atgtgacagc |
| 12061 | taggcacgtg ctagtcaaca gcccaaatcc tatgctaaag gggttgtttc acagtgaaag |
| 12121 | ccaggaagag gatgagaccc tagccagttt cctgatggat agaggagtga ttatacctag |
| 12181 | ggccgctcat gagatcttag acaacactat taccggtgca cgagaggcaa ttgctgggat |
| 12241 | gctcgacacc accaaagggc ttattcgggc tagcatgcaa agaggaggcc taactccaag |
| 12301 | gataatcacg cgattatcca catatgacta tgaccaattc aaggctggta tgagattgtt |
| 12361 | catgggtaaa ggagtcaatt tgttaattga tcaggattca tgttcggtcc agctcgcccg |
| 12421 | agcacttaga aatcacatgt gggcaacttt ggccaaagga agaccaatat acggattaga |
| 12481 | agtgcctgat atcttggaat caatgaaggg ccaattaatc atgaggcatg agtcatgtaa |
| 12541 | cctatgtatt cagggatcgc aaaattacgg atggtttttt gtgccatcaa attgccaact |
| 12601 | cgataacatt acatcaggca catctagcct tagggttcca tatatcggat ctaccacaga |
| 12661 | ggagagaacg gatatgaagc tgtcatttgt caagtcaccc agccgatcac ttaaatcagc |
| 12721 | tgtaagaata gccaccgtat actcttgggc ctacggggat gatgagaaat cttggaatga |
| 12781 | ggcatggcag ctggccaaac agagagctaa tattacactc gaggagctca ggatgattac |
| 12841 | acctgtatca acatccacga atctggccca caggctgagg gataagagca cccaagtcaa |
| 12901 | gtactccggg acatcattga ttcgagtggc aaggtatact accatttcta atgataatct |
| 12961 | ctctttcacg attgctgaga aaaaagtaga caccaacttt atatatcaac aagggatgct |
| 13021 | tctcggctta gggatcctcg agcacctatt tcgcctggtt gataacacag ggcctgcaaa |
| 13081 | cacagtcctc catttacatg tagagactga gtgctgtgtc atccccatga ccgactatcc |
| 13141 | gagtgtacca agtctccgcc aacttcggca gtcccaaaca gtttgtacaa accctctgat |
| 13201 | ctatgataag acccctataa ttgaaaagga tgcaatcagg atttacaatc aacaccaccg |
| 13261 | gcgccaggtt gtagagtttg ttacatggtc cacaaatcag ctgtatcacg tgttagccaa |
| 13321 | atctactgcc atgtctatga ttgaactcgt aactaaattt gaaaaggatc acctcaatga |
| 13381 | gatcacgacg ttgatcgggg atgatgatat caatagcttt ataactgagt tcctgcttgt |
| 13441 | cgagcctaga ttattcgttg tttatttagg ccagtgtgca gcccttaatt gggcctttga |
| 13501 | gatccacttc caccgcccga tgggtaaata tcaaatgggt gaattattgt tcacctttct |
| 13561 | aactaggatg agcaaagggg tattcaagat tctcaccaac gcattaagtc acccccgggt |
| 13621 | ttatcgaaga ttctgggaca gtgggatgat tgagccagtg tatggaccat cattggatac |
| 13681 | acaaaatctg catcaaactg tttgcaacct catctataac tgctatgtca cttacttaga |
| 13741 | ccttcttctc aatgatgata tcgataatta ccctgttgtt ttatgtgaaa gtgatgagga |
| 13801 | ggtggtgtca gatcgatttg ataacattca ggctagacac ttatgtataa tcacagatct |
| 13861 | ttattgtgat acaagagatt gcccacctat cagaggtctc tcccccgcag agaaatgcaa |
| 13921 | aacactgctt aaatttctga ggacccaagc acaattagac ccgtccggtt tatcttggaa |
| 13981 | tgatcaaccg attttagtag atcaatattc ctgctcttta acctacctta gaagaggatg |
| 14041 | cataaagcaa atcaggctaa gggtagatcc tgggtttgtg tcagctgcac tctcagaatc |
| 14101 | caaagatccc cctcgcatta tgaggtcagg caaacaggat acccagatga gcctaggact |
| 14161 | tgaacccccc ttagacgatt tactatcatt aatgggatct attaacacaa atgcacacaa |
| 14221 | tattcccgtg ataggggaag gtctccgaaa ttacgaaatc catgcattta gaaggttggg |
| 14281 | tgtaaactca acagcatgtt ataaagcagt agaaattgca tcattgatcc gctgccattt |
| 14341 | tgaagaaggt gaccaagggc tgtttctcgg ggaagggtct ggagcaatgc tggttgcata |
| 14401 | taaggaaatt ctaaaattag gcagatgcta ttataacagt ggtgtctctc ctgattcccg |
| 14461 | aactggtcaa cgggaactat ccccgtaccc atcagaagtg agtatggttg agcataaatt |
| 14521 | atgtcttgaa aaatcagtta gggtcctgtt caatggtcga cctgaagtaa cctgggtagg |
| 14581 | tagtgtagat tgttataagt atatcttaag ccagatacct accggtagta tagcggttat |
| 14641 | acactccgat attgagagcc tccctgacaa agacgctgtt gagaaactag aagagctttc |
| 14701 | tgcaatctta tcgacggccc tgatattagg caaggttggc tcagttttga ttatcaagat |
| 14761 | aatgcccatt agcggggact gggtacaggg tttcataaaa ttcgcactcg tgcattatct |
| 14821 | taaagggata ataatttacc cgaggtacag taattatgtg tcaactgagg cctatttagt |
| 14881 | attccaagga atgcgagcta gcagactggt taatcccgag ggaatcaaac agcagattat |
| 14941 | gaggatagga gtaaggacaa cacctggatt ggtatgtcat atcctctcca gaaaacaaga |
| 15001 | cacgtgtgtc caagcctaca tgggagtacc gtttaccaac gaagggttca atcctaatct |
| 15061 | gaggtcccta acacctattg agaaagtctt ggtgaactgt ggtttttctg tcaatggaga |
| 15121 | caaggtctgt cgcactctgc tacatcatga tatagtgtca gggtgcgaag gtttaacaaa |
| 15181 | tgcagtgata atcctatatc gagagctggc aaggtttaag gaaatccata cgacatccca |
| 15241 | tgggatcttt catgcatacc cagtgctaat tgagagtcaa cagcgggagt taatactaga |
| 15301 | tatcaccaga aagataaccg gccatattat cctgtactct agtaatctca ctgacattgc |
| 15361 | tcaagtaatc aagaacctca aatccggata tttaacgatt gatctattcc aaaaccaatt |
| 15421 | cacgagaaac atctccaagt cggatcgtgc actgattcgg ggaacaatag ctcgcaggaa |
| 15481 | ctgggtgttt caccttgaga caaaagaaat caaagagtgg tttaaattag tggggtatag |
| 15541 | tgctctgatt cgagagtgat ctatctactt tctggaccct cacccacatc cttgtattta |
| 15601 | gtgaaccgca aaattgacta aaaaaaacaa aaagaagaaa aaagttccct tagccaattt |
| 15661 | tgttt |

Nucleocapsid Protein (“N protein″)
SEQ ID NO: 45
CDS 62 . . . 1633
/codon start = 1
/product = “nucleocapsid protein”
/translation = “MASLLKTLSLFKKARDQPPTASGSGGALRGIKHVVIVPIPGDSTIETRSKLLDR
LIKIIGEPDTSGPKQTGALIGLLSLFVESPGQLIQRIVDDPDISIKLVEVVPSDESKSGLTFASRGANLD
AEADQYLGEDEYSSEEGAVQGWFENNEIRSVEVGDAEYFNIILASVLAQVWILLAKAVTAPDTAADSEMR
RWVKYTQQRRVIGDFRMSRLWLDSVRNRIADDLSLRRFMVALILDIKRSPGTKPRIAEMICDIDNYIVEA
GLASFILTIKFGIETMYPALGLHEFAGELTTIESLMILYQKMGETAPYMVILENSIQNKFSAGSYPLLWS
YAMGVGVELENSMGGLNFGRTYFDPAYFRLGQEMVRRSAGKVSTSFAAELGISKEDAQLVSEIATRATDE
RVGRSTGVKQTQISFLHAEKPGKGIQSEDAIIAEGRPKKTRKKTSDRLSSDDIDDDVVGPTTSFDDGRRT
DQDPDLDESSYDEKSAEAIAKMRLMTKLLGKTSPDTPTFRTYNDRELLN″
phosphoprotein
SEQ ID NO: 46
CDS 1755 . . . 3278
/codon start = 1
/product = “phosphoprotein”
/translation = “MAEEQAQHVSKGLECIKAIRDNPLCIEEIQEIKIIRDTVFNERESTVDSDQGEG
DLDEDLDESQTATSGPARKGSVQSDPHGTRSMDGPHFKGSDGEGVGISTRSDIQHYYVHSHGGEEVTGIQ
DADSLMVPTDNNRGPDDDEGTDSADEGIDESSEDDPSKPTAHDRGFAPTPAATRSADVSTVMDDELTLLL
GAHTKKKSREDPGRQLSFPKQPRIEPLEPRPESIKKGTGVKLISSGMAIDSTSINGATPSALGSPAKSSE
LSVPAANVPKSVPTAKMTLRSGPESGMHTQSEPAPVNEEEEEYEDELLAEILELKDAVTKINEDNQLILS
KLDAVLSLKGEIDSIRKQLNKQNIAISTIEGHISSIMIAIPGFGKPDPDGLAESDVNPELRPIIGRDSGR
ALAEVLKKPPVTTGKTAQPSIKSGSKRQLLKEMRLQPINPRTSSAIKFNPGDDVPSKSVISSLIKSSSLD
QSHKANMLTLLKEIKGDKNVKEFHQMVLEIIRA”
matrix protein
SEQ ID NO: 47
phosphoprotein CDS 3386 . . . 4393
/codon_start = 1
/product = “matrix protein”
/translation = “MTEVYDFDQSCWDSKGSLAPIFPNTYPDGRLIPQVRVIDPGLGDRKEECHMYLF
LLGIIEDSQNEGPSTGRAFGSLPLGVGRSMAKPEELLKEAITLDIVVRRTAGVNEKLVFYNNTPLNVLLP
WKKVLINGSIFNANQVCGGVNLIPLDVSQRFRVVYMSLTRLSDDGCYRVPRGMLDFRSGNAIAFNILVTI
QVNTVQTSGGDYISAPRGTIATFMVHIGNFKRKTNKQYSADYCKMKIEKMGLVFALGGIGGTSLHVRCTG
KMSKALNAQLGLKKILCYPLMEVNEDLNRQLWRAECSITRVQAVLQPSVPNDFRIYNDVIINDNQGLFKI
L”
putative fusion protein
SEQ ID NO: 48
CDS 5228 . . . 6901
/codon_start = 1
/product = “putative fusion protein”
/translation = “MVKPKTALCPPTGIILEALLGFILVELSYSQVHWGNLSKIGVLSTESMTYKIMA
PGASQYLVIKLMPNVTGLDNCTSSEVAEYESLLVNILGPINKALQVMTTSMRPAQTTYGSRRQKRFAGVV
LAGAALGVATAAQITAGVALHQSNMNAQAIRDLKNSLEKSNQAIEEIRQATQEVVVAVQGVQDYINHELI
PTISQMSCEIIGQKLGLKLLRYYTELLSIFGPSLRDPLSAEITIQALTYALGGDIYKVIEKLGYSGHDLA
AVLESGGIKTRVVHADVQSKFIILSISYPTLSEVKGVVVHKLDVVSYNIGSQEWYTAVPKFVATNGYLIS
NFDESGCALVTGVSICSQNALYPMSSVMQQCMRGETGSCARTLVSGTIGNRFILSKGNIVANCASIMCKC
TSGTIINQDPDKLLTFIATDLCPLVEIEGITIQVGTREYPDTVYEHRINLGPVIPLEKLDVGTNLGNAIS
KLEDAKDLLDASNQILDLIQDSRMSIGKYIIWAAVGFSIFGGLLLLCCCKRYYQKVSANSTVINPTVKPD
LTGTSRSYVKSL”
hemagglutinin protein
SEQ ID NO: 49
CDS 7057 . . . 8874
/codon_start = 1
/product = “hemagglutinin protein”
/translation = “MPPLGDKSNAFYKDSARANLPILNINGFLPAHHRGPLLLLLATVSLVGLVALIA
IAGVRFHKSTTMAKETNSLIQENIHHTDLLHHQVMDVLTPLFKIIGEEVGLRLPQRLAEIKQFILTKTSF
FNPDREFDFRDLHWCINPPDRVKVNFSKFCQAIHIDNGIRLLGGIFIDQFLSHSKSDIFPGRVCPNGVTT
RGEIISPALSLTSLSAKHLGLVDNIMFAMADGVYAKTYVLSHFSEGGVTETGEIRVFEIGLIRGWLGDLP
VLHLTNFIHANVDSIGRHCTLAVGELKLASICTHARAIRVVRNVDSHPTSVSAISMGVFGSDYYQQSVEV
IPVYDESVQQIHLSNHRGFIKDSKAYWAFPAEADTNSTRWSNCLTKVCTSRNLPFCNLTSWEPFNTTYPA
VYAILVLEVSVHSDISMFIDESLGPLILNGYGMDLYSNSHEDQHWLTIPPKSGNVLGVINKLVVNGSAAV
LPYILSYAASAGHEPCYAPVQHSLPSDTNTLAESNIVVLSTGKFRYVSATYDTSRSSHAIVYYIYNPTEG
TSYFFPFRLTTRGIPAFLRIECFVWVEKIWCIHVYRYHSTVSKVEPVVETMVRLEFKCTSS”
large protein
SEQ ID NO: 50
CDS 9008 . . . 15559
/codon start = 1
/product = “large protein”
/translation = “MDSISVNQILYPEVHLDSPIVINKLVAILEYANLNHNYQLIDPTLIENIKQRIN
EGYSNQMIINYINVGHIINDLLATLPKTTHILYPNCNKDLFRAEDALVTQKLLDLFKKGNTIYTKISGGI
IRCLTNLNQSLGLGGNLPEIYHNPIQDLGVMMQSSKWFGSFLFWFTIKTEMRSVIKSSTHNCQKRRYIPS
FTTGKNFKILVSRDLVCLVNLADKRVYYLTFEMVLMYCDVIEGRLMTDTAMCIDSKFAQLRKRVHYLWDL
IDGFFPDLGNSTYQLVALLEPLSLAYLQLKDATDQLKGAFLSHCFTEIKEILDNQGFTLPDTYAQLIEAL
DTIFITDDIHLTGEIFSFFRSFGHPRLEAVTAAENVRKHMNQPKVISYETLMKGHAIFCGIIISGFRDRH
GGSWPPLILPEHASPVVKNAQASGEGLTYSQCVDNWKSFVGMRFKCFMPLSLDSDLTMYLKDKALSALRK
EWDSVYPKEYLRYNPPKSSGSRRLVNVFLEDSEFDPYNMIMYVISGEYLRDPDFNLSYSLKEKEIKEVGR
LFAKMTYKMRACQVIAENLIANGIGKYFKDNGMAKDEHDLTKALHTLAVSGVPKDKSQKHRDPRTKIDNH
YNRETGGLPKCQLGGKQGPSGHRTGFISGRADDNDIDLYETVSAFITTDLKKYCLNWRYETISIYAQRLN
EIYGLPSFFEWLHKRLEKSVLYVSDPHCPPDLDHHINLDEVPNQQIFIKYPMGGIEGYCQKLWTISTIPY
LYLSAHECGVRIASLVQGDNQTIAVTKRVPSSWPYHLKKAEAARVTINYFMILRERLHDIGHHLKANETI
ISSHFFVYSKGIYYDGMLISQSLKSVARCVFWSETIVDETRAACSNISTTLAKSIEKGFDRYLAYALNLI
KTIQQLLISLGFTINGSMTDDVVEPLLKDHRIITKMALLPAPIGGFNYLNMSRLFVRNIGDPVTSSLADL
KRMIKANLLSETILHQVMTQAPGDSSFLDWASDPYSANLPCTQSMTRVLKNVTARHVLVNSPNPMLKGLF
HSESQEEDETLASFLMDRGVIIPRAAHEILDNTITGAREAIAGMLDTTKGLIRASMQRGGLTPRIITRLS
TYDYDQFKAGMRLFMGKGVNLLIDQDSCSVQLARALRNHMWATLAKGRPIYGLEVPDILESMKGQLIMRH
ESCNLCIQGSQNYGWFFVPSNCQLDNITSGTSSLRVPYIGSTTEERTDMKLSFVKSPSRSLKSAVRIATV
YSWAYGDDEKSWNEAWQLAKQRANITLEELRMITPVSTSTNLAHRLRDKSTQVKYSGTSLIRVARYTTIS
NDNLSFTIAEKKVDTNFIYQQGMLLGLGILEHLFRLVDNTGPANTVLHLHVETECCVIPMTDYPSVPSLR
QLRQSQTVCTNPLIYDKTPIIEKDAIRIYNQHHRRQVVEFVTWSTNQLYHVLAKSTAMSMIELVTKFEKD
HLNEITTLIGDDDINSFITEFLLVEPRLFVVYLGQCAALNWAFEIHFHRPMGKYQMGELLFTFLTRMSKG
VFKILTNALSHPRVYRRFWDSGMIEPVYGPSLDTQNLHQTVCNLIYNCYVTYLDLLLNDDIDNYPVVLCE
SDEEVVSDRFDNIQARHLCIITDLYCDTRDCPPIRGLSPAEKCKTLLKFLRTQAQLDPSGLSWNDQPILV
DQYSCSLTYLRRGCIKQIRLRVDPGFVSAALSESKDPPRIMRSGKQDTQMSLGLEPPLDDLLSLMGSINT
NAHNIPVIGEGLRNYEIHAFRRLGVNSTACYKAVEIASLIRCHFEEGDQGLFLGEGSGAMLVAYKEILKL
GRCYYNSGVSPDSRTGQRELSPYPSEVSMVEHKLCLEKSVRVLFNGRPEVTWVGSVDCYKYILSQIPTGS
IAVIHSDIESLPDKDAVEKLEELSAILSTALILGKVGSVLIIKIMPISGDWVQGFIKFALVHYLKGIIIY
PRYSNYVSTEAYLVFQGMRASRLVNPEGIKQQIMRIGVRTTPGLVCHILSRKQDTCVQAYMGVPFTNEGE
NPNLRSLTPIEKVLVNCGFSVNGDKVCRTLLHHDIVSGCEGLTNAVIILYRELARFKEIHTTSHGIFHAY
PVLIESQQRELILDITRKITGHIILYSSNLTDIAQVIKNLKSGYLTIDLFQNQFTRNISKSDRALIRGTI
ARRNWVFHLETKEIKEWFKLVGYSALIRE”

The disclosures of each and every patent, patent application, and publication cited herein are hereby incorporated herein by reference in their entirety.

While the invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. An isolated nucleic acid molecule comprising a nucleotide sequence selected from the group of:
a nucleotide sequence comprising a genome sequence of porcine morbillivirus of SEQ ID NO: 4;
a nucleotide sequence which encodes at least 5 contiguous nucleotides SEQ ID NO: 45, 48, or 49;
a nucleotide sequence having at least 90% sequence identity to a SEQ ID NO: 4; and
a nucleotide sequence encoding the amino acid sequence of porcine morbillivirus nucleocapsid protein, phosphoprotein, matrix protein, fusion protein, hemagglutinin protein, and/or large protein.

2. A nucleotide construct comprising the nucleic acid molecule of claim 1 operably linked to a promoter.

3. A vector comprising the construct of claim 2.

4. A cell comprising the vector of claim 4.

5. The composition of claim 4, wherein the recombinant expression vector is a baculovirus expression vector.

6. A method of producing porcine morbillivirus, comprising:
transfecting a host cell with the isolated nucleic acid molecule of claim 1 or a vector comprising the isolated nucleic acid molecule of claim 1;
allowing replication of polynucleotide or vector in the host cell; and
harvesting the produced porcine morbillivirus from the medium and/or cells.

7. An immunotherapeutic comprising a porcine morbillivirus epitope.

8. The immunotherapeutic of claim 7 comprising the nucleic acid molecule of claim 1.

9. The immunotherapeutic of claim 7, wherein the epitope is on an attenuated virus, an inactivated virus, a protein subunit, a virus-like particle, a viral vector, a nucleic acid vaccine, or an artificial antigen-presenting cell.

9. A method for protecting a pig against porcine morbillivirus respiratory and gastrointestinal disease comprising: administering to the pig the composition of claim 7.

10. The method of claim 9, wherein the composition is administered via a route selected from the group consisting of intravenous, intramuscular, intradermal, subcutaneous, and oral.

11. A method for aiding in the prevention or reduction of one or more clinical symptoms associated with infection with porcine morbillivirus respiratory and gastrointestinal disease, said method comprising administering the composition of claim 7 to a pig.

12. The method of claim 11, wherein the clinical symptoms are selected from the group consisting of encephalitis, placentitis, nonsuppurative myocarditis, coughing, dyspnea, polypnea, increased mortality rate, fetal death, neuronal necrosis, neuronal and glial mineralization, neuronal viral inclusion bodies, lesions of the ganglia, and any combination thereof.

13. A method of producing antibodies against porcine morbillivirus, comprising:
administering to a population of B cells an epitope of porcine morbillivirus;
allowing antibody production; and
harvesting the produced antibodies.

14. The method of claim 13, further comprising forming a hybridoma.

15. A method of detecting porcine morbillivirus or a nucleic acid or protein thereof in a sample, comprising:
obtaining a sample from a subject;
adding a molecule capable of binding to the porcine morbillivirus or a nucleic acid or protein thereof; and
detecting the binding of the molecule to the porcine morbillivirus or a nucleic acid or protein thereof.

16. The method of claim 15, wherein the molecule is a DNA primer or probe.

17. The method of claim 16, wherein the molecule is an antibody.

18. A method of detecting porcine morbillivirus (PoMV) in a sample, the method comprising:
contacting the sample with a PoMV polypeptide under conditions that allow the formation of an antigen-antibody complex between said PoMV polypeptide and a PoMV antibody present in the sample, wherein the PoMV polypeptide comprises a fragment of at least 5 amino acids and wherein the PoMV peptide comprises at least 90% identity to the amino acid sequence of SEQ ID NOs: 45, 48, and/or 49; and detecting the antigen-antibody complex formed.

19. The method of claim 18, wherein the sample is serum, whole blood, or biopsies.

20. The method of claim 18, wherein the method comprises:
providing a well of a microtiter plate, wherein the well comprises the PoMV polypeptide;
introducing into the well the pig sample under conditions that allow the formation of the antigen-antibody complex;
subsequently introducing into the well an enzyme-labeled antibody directed against pig immunoglobulin, wherein the enzyme is capable of hydrolyzing a substrate;
adding the substrate to the well; and
detecting hydrolyzed substrate, thereby detecting the presence of PoMV antibody in the pig sample.

21. The method of claim 18, wherein the detecting of the antigen-antibody complex formed is by the ELISA technique, by immunofluorescence, or by radioimmunological processes (RIA) or their equivalent.

22. The method of claim 18, wherein the PoMV polypeptide is selected from the group consisting of SEQ ID NO: 45, SEQ ID NO: 46, SEQ ID NO: 47, SEQ ID NO: 49, SEQ ID NO: 48, and SEQ ID NO: 50.

23. An amino acid sequence encoding porcine morbillivirus (PoMV) nucleocapsid protein, phosphoprotein, matrix protein, fusion protein, hemagglutinin protein, and/or large protein.

24. The amino acid sequence of claim 23, comprising one or more of SEQ ID NOs: 45, 46, 47, 48, 49, and/or 50.

25. The nucleic acid sequence of claim 23, wherein said PoMV polypeptide comprises one or more amino acid substitutions so that said PoMV polypeptide is not naturally occurring.

26. The amino acid sequence of claim 25, wherein said substitution affects an N-glycosylation site, and wherein the site comprises an amino acid deletion or an amino acid substitution.


SEQUENCE LISTING (50 sequences)

SEQ ID NO: 1 - DNA, 20 bp, Artificial
  synthetic
accaaacaaagttgggtaag

SEQ ID NO: 2 - DNA, 23 bp, Artificial
  synthetic
gggctagcaagctttatcctcac

SEQ ID NO: 3 - DNA, 24 bp, Artificial
  synthetic
gttaactcaccagcaaactcatgc

SEQ ID NO: 4 - DNA, 19 bp, Artificial
  synthetic
atgaatcacagacggcaac

SEQ ID NO: 5 - DNA, 26 bp, Artificial
  synthetic
aacatagtagtgctgtatgtcagatc

SEQ ID NO: 6 - DNA, 20 bp, Artificial
  synthetic
catttggtggaattccttga

SEQ ID NO: 7 - DNA, 21 bp, Artificial
  synthetic
gataaagtcaagcagcctgga

SEQ ID NO: 8 - DNA, 17 bp, Artificial
  synthetic
gtcactgcttggccctc

SEQ ID NO: 9 - DNA, 26 bp, Artificial
  synthetic
ccaggggctgttcaagatcctataag

SEQ ID NO: 10 - DNA, 30 bp, Artificial
  synthetic
gccggtcacattaggcataagttttataac

SEQ ID NO: 11 - DNA, 19 bp, Artificial
  synthetic
tacaaaattatggcacccg

SEQ ID NO: 12 - DNA, 25 bp, Artificial
  synthetic
cataaacagtgtctgggtactctct

SEQ ID NO: 13 - DNA, 24 bp, Artificial
  synthetic
ttacttacattcatcgcgactgat

SEQ ID NO: 14 - DNA, 28 bp, Artificial
  synthetic
ctagtaagagataaggcaggtgatataa

SEQ ID NO: 15 - DNA, 30 bp, Artificial
  synthetic
cttatcacatagcaaatcagatatattccc

SEQ ID NO: 16 - DNA, 24 bp, Artificial
  synthetic
caccatattttctcaacccataca

SEQ ID NO: 17 - DNA, 29 bp, Artificial
  synthetic
catgcaatagtgtattatatctacaatcc

SEQ ID NO: 18 - DNA, 20 bp, Artificial
  synthetic
taagcgagggaaagtggttc

SEQ ID NO: 19 - DNA, 20 bp, Artificial
  synthetic
ctctgggatctgattgacgg

SEQ ID NO: 20 - DNA, 29 bp, Artificial
  synthetic
cttacagtctcataaaggtctatgtcatt

SEQ ID NO: 21 - DNA, 21 bp, Artificial
  synthetic
gtctcccaaaatgtcaacttg

SEQ ID NO: 22 - DNA, 20 bp, Artificial
  synthetic
ctgttgactagcacgtgcct

SEQ ID NO: 23 - DNA, 20 bp, Artificial
  synthetic
ttagattgggcaagtgatcc

SEQ ID NO: 24 - DNA, 18 bp, Artificial
  synthetic
tgtttgggactgccgaag

SEQ ID NO: 25 - DNA, 23 bp, Artificial
  synthetic
aacacagtcctccatttacatgt

SEQ ID NO: 26 - DNA, 23 bp, Artificial
  synthetic
cttctaaatgcatggatttcgta

SEQ ID NO: 27 - DNA, 29 bp, Artificial
  synthetic
gacgatttactatcattaatgggatctat

SEQ ID NO: 28 - DNA, 22 bp, Artificial
  synthetic
aaacaaaattggctaagggaac

SEQ ID NO: 29 - DNA, 23 bp, Artificial
  synthetic
gggctagcaagctttatcctcac

SEQ ID NO: 30 - DNA, 24 bp, Artificial
  synthetic
gttaactcaccagcaaactcatgc

SEQ ID NO: 31 - DNA, 29 bp, Artificial
  synthetic
cagtgccgggtacatagtttctatcccaa

SEQ ID NO: 32 - DNA, 28 bp, Artificial
  synthetic
gactgtwgagactaaaggtggaactgta

SEQ ID NO: 33 - DNA, 21 bp, Artificial
  synthetic
gcttctacacctgggacagca

SEQ ID NO: 34 - DNA, 14 bp, Artificial
  synthetic
cccgttggaatggt

SEQ ID NO: 35 - DNA, 20 bp, Artificial
  synthetic
tgtwcgggcacacagccata

SEQ ID NO: 36 - DNA, 21 bp, Artificial
  synthetic
tttccgcataagggtcgtctt

SEQ ID NO: 37 - DNA, 18 bp, Artificial
  synthetic
accacaaacacttggctc

SEQ ID NO: 38 - DNA, 21 bp, Artificial
  synthetic
ccaaaaatgcaaaccccaata

SEQ ID NO: 39 - DNA, 21 bp, Artificial
  synthetic
tctggcggtgttggagttaag

SEQ ID NO: 40 - DNA, 21 bp, Artificial
  synthetic
cttggagccgtggagcgagcc

SEQ ID NO: 41 - DNA, 20 bp, Artificial
  synthetic
aagcattaccgcttgtggtg

SEQ ID NO: 42 - DNA, 20 bp, Artificial
  synthetic
gaactcccatttcagcgatt

SEQ ID NO: 43 - DNA, 19 bp, Artificial
  synthetic
aaagccaggacaagcgccg

SEQ ID NO: 44 - DNA, 15665 bp, Artificial
  synthetic
acttaccttaggatccatgatctgatcgccaagagtgggtgcagggatctgagacctagtcatggctagcttactaaagactttatcacttttcaagaaggcacgtgatcagccccctacagcatctgggtcaggaggagcattaagaggtatcaaacatgtggtaattgtcccaattcctggtgactctacgattgagactcgatcaaagctcttggaccgactgattaagatcatcggggagcccgatacaagtgggccaaagcaaaccggagcactaatcgggctgctatcactttttgtggagtccccaggacaattgatccaaagaatcgttgatgatcctgatatcagtatcaaattggttgaggtggtcccaagtgatgagtcaaaatcaggtttgacgtttgcttctcgaggtgccaatttggatgctgaggctgaccagtacttaggagaagatgagtactcaagcgaggaaggggctgtacaaggttggtttgaaaataatgaaatcaggagtgttgaggtgggcgatgctgaatatttcaacatcatccttgcatcagtattggcccaagtttggatacttctggcgaaggccgtaaccgccccggatactgctgcagactccgaaatgagacgctgggtcaagtatacccagcagaggcgggtcatcggtgactttagaatgtcacgtttatggttagactcagtcaggaacagaattgctgatgatctgtctctccggagattcatggtggcattgatcttggacataaagagatctcctgggacgaagcccagaatcgctgagatgatctgtgacattgacaactacattgtagaagccgggctagcaagctttatcctcacgatcaagtttgggatagaaactatgtacccggcactgggcttgcatgagtttgctggtgagttaacaaccattgaatccctgatgattctataccaaaagatgggtgaaaccgctccttacatggtcatactagaaaattccattcagaataaattcagcgcagggtcataccctctactttggagctacgccatgggggtaggtgttgagctggagaattccatgggggggcttaattttggtagaacatattttgatcctgcgtatttcagacttggacaagagatggtacgtagatctgctggaaaggtcagcacctcttttgctgctgagttgggaatatcgaaagaggatgcgcagctggtttcagaaattgccaccagagcaaccgatgaaagagttgggaggtccacaggagtcaagcaaacccagatatcctttctgcacgctgaaaaaccggggaaggggattcaatcagaggatgccattattgctgaaggtcgcccaaagaaaaccagaaagaagacatctgatcgtctcagttccgatgacatcgacgatgatgttgtgggaccaactaccagctttgatgatggtcgacggacagatcaggaccccgacttagacgaatcctcctatgacgagaaatctgctgaagctatagcaaagatgcgtctaatgactaaattgctaggaaaaacctccccagatactcccacattccgcacctataatgacagagaactactcaactgaccaagcaatgcccatcagcccattctaccactgaccactcccaactgcattataaaaaacttaggacccaggtccacaaatcacagatcacaaccacagataacaccagtcaccctctccaatggctgaagagcaggcccagcatgtcagcaaagggctggagtgcattaaagccatcagagataatcctctctgtattgaggagattcaggaaatcaagatcatccgcgacaccgtcttcaacgaacgtgaatctaccgtcgactctgatcagggagaaggggatctcgatgaggatctcgatgaatcacagacggcaacaagtggcccagcaagaaagggttcagtacaatcagaccctcatggaactagatcaatggatggaccacattttaagggatcagatggtgaaggagttggtatcagcacaagatctgacatacagcactactatgttcacagccacggcggtgaagaggttacgggaatccaggatgctgacagtctcatggtaccaacagataacaaccgtggtcccgatgacgacgagggaacggatagcgctgatgagggcattgatgaatctagtgaggatgatccctcaaaacctactgcccatgacaggggatttgctcccactcctgctgcgactagatcagcagacgtgtcaaccgtgatggatgacgaactaaccttgctgttgggggctcataccaagaagaagagcagagaggacccggggagacagctgagttttccgaaacaacctagaattgaacccctcgagccgagaccagaatctattaaaaagggcacaggcgtgaaattaatctcatctggaatggcgatcgactccacatcgatcaatggtgcaaccccatctgcgctcgggtcaccagcgaaatcatcagagctcagtgtacctgcggcgaatgtccccaagtctgtcccgactgcaaaaatgaccctgagatccggacccgagtctggtatgcatactcagtcggagccagccccggtcaatgaagaggaggaagagtacgaagatgagcttcttgcagagatactagagttgaaagatgctgtcaccaaaatcaatgaggataatcaactgattctgtcgaaattggatgctgttctgagcctcaagggagagattgattccatcagaaaacaactcaacaaacaaaatattgcaatatcgactatcgaaggacatatttcaagcattatgatcgcaatacccggattcggtaaacctgacccagatgggcttgcagaatcagacgtgaatcctgagctccgcccaataattggtagagactcaggacgtgcattggccgaagttcttaagaaacccccagtcaccaccgggaaaacagcgcaaccctccatcaagtcaggttccaagcgccagctgttaaaggagatgaggcttcaacccatcaacccacgaactagctctgcaatcaagttcaacccgggggacgacgtcccctctaagagcgttattagttccctgataaagtcaagcagcctggatcagagccacaaggccaatatgttaactcttctcaaggaaatcaagggagacaaaaacgtcaaggaattccaccaaatggttctggagattatcagggcttaatagcaatgcttcttcagcataaccccccaatataactcgctcaagcataggtgtagcatgcattataaaaaacttaggatcaaagttgatccatcagtaactcaaaaatgacagaagtgtacgactttgaccaatcgtgctgggattcaaaggggtccctcgccccaatcttcccgaatacttatccggatggtcgattgatacctcaggttcgggtcatagaccctgggcttggcgacagaaaggaagaatgtcacatgtatcttttcttgttggggataattgaggacagtcaaaatgagggcccatcaacagggcgggcattcggatccctcccgttaggtgttggtagatctatggcaaagccggaagaactactcaaggaggcgataaccctggatattgtagttcggaggacagcaggagtgaatgagaagctagttttttataacaacacccctttgaatgttctcctgccatggaaaaaagtcttgaccaatggaagcatctttaacgcgaaccaggtctgcggcggagtgaaccttataccgctggatgtgtcacagagattcagagtggtgtatatgagccttactcgtctgtcagatgatgggtgttatcgagtgcctagggggatgctagattttagatctgggaatgcgattgctttcaatatactagtaacgattcaagtcaacactgttcagacaagcgggggtgattatatcagtgcaccaagagggaccatagccaccttcatggtccatattgggaactttaaacggaagacaaataagcagtattcagctgattactgcaagatgaagatagaaaaaatgggactagtatttgctcttggtggaatcggaggtaccagtctccatgtgaggtgcacagggaagatgagtaaagctcttaatgcccaattaggcttgaaaaagattctgtgttatccgttgatggaagttaatgaggatctcaatagacagctctggagagctgagtgcagcatcacccgtgtccaggcagtcttgcaaccatctgttccgaatgactttcgtatatacaatgatgttatcatcaacgataaccaggggctgttcaagatcctataagagggatagaatatgtgcattttcaacagcatgcccaggccttgcacccgcggcacacaactcagccctctcccccggtcgagggccaagcagtgaccggcatgtgcacccaaggtcaaaagcacccctcaggaccatcacccccgatacagactccacaagtgcccatcacaacacagtacactcacaagcacacatcctccccacagggatccaaaagcaccccactccccaaatcggcaagccgaccccatctcacatctcaacctcaaatcacacacccccaggacgtagcactatctgagggaaccgcacaatgcccattctacactcaccgccatagagcctccaactagctcgctcccgcactttctctacttagtcgatagattgagcattaagaaaaactgaggacccaagtcccacatcagcccggactcccaccgtccaccagggcccacggccagaccaacgagcagaccaacggaagcaccaccagaacccgagagggagcagcgcaggtccaaggcagggccaggtcaagcgctgcacccccccgggcagaccccccccatccctccccgagaacccagaccgcacagactgaacaggtccgcatatccaatcgcgcaaccccgagacaacccgggagccctcacgaccgaccttccgtccgtccgacccaccaaatcctgtgccccaggacagctcgccaacaaaccctccattagaaacccgaccaccaccccactcccctccccggcggaccgaaccaagagaagccctctcaatctcccctgaaggtgccaggctgccttccccctccacctcgccatggtcaaaccgaaaacagccctttgcccacccacaggcataatccttgaagctctcttgggattcatactagtggaattatcgtatagtcaagtacactggggtaatctgtctaagataggagtcctgagcactgaaagcatgacatacaaaattatggcacccggggcgagccagtatttagttataaaacttatgcctaatgtgaccggcttagacaattgtactagcagtgaagttgctgagtacgaatctttactggtgaacatccttggccccatcaataaagctttgcaggtaatgacaaccagcatgaggcctgcccagactacatacgggtcacgtcgacagaaaaggtttgccggtgttgtccttgcaggggcagcattaggagttgccacagcagctcagataacggctggggtagcattacaccagtcaaatatgaatgcccaggctattcgggatcttaaaaatagcctggagaaatccaatcaagcaattgaagaaattaggcaggctacacaggaagtagtcgtagccgtacagggggtgcaggattatataaatcatgaacttattccaacaatcagccaaatgtcctgcgagatcataggacagaaattagggctaaagctgcttaggtactatactgagttgctgtctatattcggccctagcctgagggaccctctctctgctgagataacaatccaggcattgacttatgccttgggtggtgatatatataaggtgattgaaaaactagggtactccggtcatgatctagcagccgttctcgaaagtgggggaataaaaacacgagtggttcacgctgatgtacaatcaaaattcataatcttgagtatttcatacccaacattatccgaggtcaagggggtcgttgtacataagttagatgtcgtttcatacaacattggctcccaagagtggtacaccgcagtccccaaatttgtcgccacgaatggttaccttatctctaacttcgatgaatcgggctgtgctttagttaccggggtatcaatttgcagccagaatgcgttgtatcctatgagttctgtaatgcagcagtgtatgaggggagaaactggatcttgtgctagaacattagtctctggaacaattgggaatagattcatactttcaaaagggaatattgtcgcgaactgtgcatcaattatgtgcaaatgcacaacttctggaactatcatcaatcaagatccggacaaattacttacattcatcgcgactgatctatgcccacttgttgagattgagggaataacaatccaggtcggtactagagagtacccagacactgtttatgaacataggatcaaccttggccctgtaatcccacttgagaaactcgatgtcggtacaaatcttggtaatgcaatctccaagttagaagatgcaaaggatcttctcgatgcttctaaccaaatccttgatcttattcaagattctagaatgtccattggcaaatatataatttgggctgctgtaggattttctatctttggcgggttgttattactgtgctgctgtaagaggtactaccagaaagtctcagcaaattctactgtcattaatcctacggtcaaaccagacttgacagggacctcaagatcttacgtcaagtcattgtgaaccctggtgcctgtttggctttgtaaagggattagaggcagcctagcctttgcgtgattggtaacccctgcactcgtcccttggattttgtaaaacttttacaccttctcaatagccaactattaagaaaaacttaggctccaagtcctacagccatgccccctcttggagacaaaagcaatgctttctacaaggattctgccagagctaatctccctatccttaacatcaatggatttctacctgcccatcaccgaggtcccctcctcttgctactggcaactgtctcactagtgggtttggttgcattgattgcaattgcaggggtgagattccacaagtctaccactatggcaaaggagactaactcgctgatccaggaaaacatccatcacactgacctactacaccaccaggtgatggatgtgttaacccctcttttcaagataatcggggaagaggttggccttagattaccacagcgattagcagagatcaagcaatttatcttaacgaagacgtcattcttcaatccagatagagaatttgatttcagagacctccattggtgcattaatcctcctgaccgagttaaggtcaatttcagtaaattttgccaggccattcacatcgacaatgggatacgactcttaggtggtatattcattgaccagttcttatcacatagcaaatcagatatattccctggtagggtatgcccaaatggggttacaactaggggagagattatatcacctgccttatctcttactagtctaagtgcaaaacatcttgggttagtcgataacattatgtttgctatggcggatggtgtctatgctaagacttatgtactttcccacttttcggagggaggggtcaccgaaacgggagagattagggtttttgagatagggttgattaggggttggctgggtgacctcccagtgcttcatctcacaaactttatccatgcaaatgtagattcaataggcagacactgcacactagctgttggggaactgaaactcgcatctatttgtacccacgcgcgggcaataagagtggtgaggaatgtggatagtcacccaacttctgtctcggcaatctctatgggtgtattcggatcagactactaccaacaaagtgttgaagtgatcccagtgtatgatgaatctgtacagcaaattcatctgtcgaaccaccgagggtttataaaggactcaaaggcatactgggcgttccccgctgaagctgacactaattccactcgatggtccaactgcttgacgaaagtctgcacctctcgtaacttacccttttgcaacctgaccagctgggagccattcaatactacatatcccgcagtgtatgccatattggtccttgaggttagtgttcacagtgatatctcgatgttcatagacgagtccctaggtccattaatcttgaatgggtatggtatggatttatactcgaacagccatgaggaccagcattggttgactatcccaccaaaaagtggcaatgttctgggtgttatcaacaagttagtggtcaatgggagtgcggcggtgctcccgtacatacttagctatgctgcatcagctggccatgaaccatgttatgcccctgtccagcactctttacccagtgacaccaacacgcttgctgaatcaaatatagttgtcctgtcaaccggaaagttcagatacgtttctgcaacttatgacacatcgaggagcagccatgcaatagtgtattatatctacaatcctacagagggaacatcctattttttccccttccgacttacaacaaggggaattccagcattcctgcgcatagagtgctttgtatgggttgagaaaatatggtgcatacatgtgtaccggtaccattctaccgtcagtaaggtagaaccagttgtggaaactatggtcagattggaatttaagtgtacctcctcttaagatccctccgttctcctttgacccttaaactggaatctagaagtttaagtaccacctgctacatgcctatccagcactccataactcttctgagagaccataaaaaaacttagggtccaggtaagtttgcactatggactctatctcagttaaccagattctctacccggaagttcatctcgacagccccatcgtgaccaataagttagtagctatcctagagtatgccaatctaaatcacaactaccagttgatagacccgacccttattgagaatattaaacagcggatcaatgaaggatactccaaccaaatgatcatcaactatatcaacgtcggacacatcataaatgatctactggctactttgcctaaaacaactcatattctgtatcctaattgtaataaagatctgtttagggcagaagatgcgttggttactcaaaaattgctagatttgttcaagaaaggaaacacgatatatacgaaaatatccggagggataattagatgcttaaccaaccttaatcaatcactgggtcttggaggcaatctccctgaaatctaccataacccaatacaagatttaggagttatgatgcaaagttcaaagtggttcggttcattcctgttttggttcaccatcaaaaccgagatgaggtctgtaataaaatcaagtactcacaactgtcaaaaaaggagatatatcccgtcattcaccaccggaaagaatttcaaaatccttgtttcgagagacctcgtttgccttgtgaaccttgctgacaagagggtttactacttaacatttgaaatggtcctgatgtactgtgatgtcatcgaaggccgattaatgactgacacagccatgtgtattgattcaaaatttgctcagctacgtaaaagggtccactatctctgggatctgattgacggattttttcctgacttagggaactctacctaccagctcgtagcgttgcttgaaccactttccctcgcttacctccagctgaaagatgcaacagatcagctcaaaggcgcctttctgtctcattgctttacagagattaaagagattttagataaccaaggttttactttgccagacacatatgctcaattaatcgaggctttagatactatctttatcactgatgatatccatctgacaggtgagattttttctttttttagaagttttggccatcctcggttagaggcagtgactgcagcagagaatgtgaggaagcacatgaatcagccaaaagtcatctcctatgaaaccctaatgaaaggtcatgcaattttctgcgggataatcattagcggattccgggaccggcatggagggagttggccccctttgattctccctgagcatgcttcaccagtagttaaaaacgcccaggcttcaggtgaagggctaacatacagccagtgtgttgacaattggaaatcattcgtgggtatgaggtttaagtgttttatgcccttgagccttgacagtgacctaacaatgtatctaaaagataaggctttatctgccctcaggaaagagtgggactctgtgtatccgaaagaatatctacggtacaacccccccaagtcctcgggatctagaagattagttaatgttttcctagaggattccgaattcgacccttataatatgatcatgtatgtaatatctggagaatacctgagagacccggacttcaacttgtcctatagtctcaaggagaaagaaatcaaagaagtcgggaggctattcgcaaaaatgacatacaagatgagagcatgccaggtgattgctgaaaacttaatcgccaatggtataggaaagtatttcaaggataacggcatggcaaaagacgaacatgacctaacaaaagcattacataccctggctgtctctggggtccccaaagataaatcccaaaagcaccgagaccccagaactaaaatagataaccactataatagagaaacagggggtctcccaaaatgtcaacttggaggaaaacagggcccatcaggacatcggacgggtttcatcagtggccgggctgatgataatgacatagacctttatgagactgtaagtgcttttataaccactgatctcaaaaagtattgtttaaattggcgttatgagacaataagcatctatgcccagaggttaaatgagatttatggactcccttctttctttgaatggttacataaaaggttagagaagtctgtactgtatgtcagcgatcctcattgcccaccagatctcgaccaccatatcaacctggatgaggtcccaaatcaacaaatctttatcaaatacccgatgggtggaattgaggggtattgccaaaagttatggaccataagcactatcccctatctttacttgtcagctcatgaatgcggagtgagaatagcttctttagttcagggagacaaccagactatagctgttaccaagcgagttccgagttcatggccataccaccttaagaaggcagaagcagctagagtcacaattaattatttcatgattttacgggaaagacttcatgatataggtcaccatttaaaagctaatgaaaccataatttcatcccatttttttgtatattcaaaagggatttactacgatggcatgctaatctcacagtcccttaaaagcgtagcccgatgtgttttctggtcagaaactattgtagatgagactagagcagcttgtagcaacatatccacaaccctggctaaatcaattgaaaaaggatttgatagatatctagcctatgcccttaaccttatcaaaaccatacagcagctgttgatttcactgggctttaccatcaatggatccatgacagacgatgtggttgaaccccttttaaaagatcatagaattataacaaagatggcactcctccctgccccgataggaggattcaattatttgaacatgagccgcctgttcgttagaaatataggcgatccggtcacatcatcattggctgatttaaaaagaatgattaaagcaaatctgctgagtgaaacaattcttcatcaagtgatgactcaagccccaggtgattcgtcatttttagattgggcaagtgatccgtactcagctaatctcccatgcactcaaagcatgaccagagtccttaaaaatgtgacagctaggcacgtgctagtcaacagcccaaatcctatgctaaaggggttgtttcacagtgaaagccaggaagaggatgagaccctagccagtttcctgatggatagaggagtgattatacctagggccgctcatgagatcttagacaacactattaccggtgcacgagaggcaattgctgggatgctcgacaccaccaaagggcttattcgggctagcatgcaaagaggaggcctaactccaaggataatcacgcgattatccacatatgactatgaccaattcaaggctggtatgagattgttcatgggtaaaggagtcaatttgttaattgatcaggattcatgttcggtccagctcgcccgagcacttagaaatcacatgtgggcaactttggccaaaggaagaccaatatacggattagaagtgcctgatatcttggaatcaatgaagggccaattaatcatgaggcatgagtcatgtaacctatgtattcagggatcgcaaaattacggatggttttttgtgccatcaaattgccaactcgataacattacatcaggcacatctagccttagggttccatatatcggatctaccacagaggagagaacggatatgaagctgtcatttgtcaagtcacccagccgatcacttaaatcagctgtaagaatagccaccgtatactcttgggcctacggggatgatgagaaatcttggaatgaggcatggcagctggccaaacagagagctaatattacactcgaggagctcaggatgattacacctgtatcaacatccacgaatctggcccacaggctgagggataagagcacccaagtcaagtactccgggacatcattgattcgagtggcaaggtatactaccatttctaatgataatctctctttcacgattgctgagaaaaaagtagacaccaactttatatatcaacaagggatgcttctcggcttagggatcctcgagcacctatttcgcctggttgataacacagggcctgcaaacacagtcctccatttacatgtagagactgagtgctgtgtcatccccatgaccgactatccgagtgtaccaagtctccgccaacttcggcagtcccaaacagtttgtacaaaccctctgatctatgataagacccctataattgaaaaggatgcaatcaggatttacaatcaacaccaccggcgccaggttgtagagtttgttacatggtccacaaatcagctgtatcacgtgttagccaaatctactgccatgtctatgattgaactcgtaactaaatttgaaaaggatcacctcaatgagatcacgacgttgatcggggatgatgatatcaatagctttataactgagttcctgcttgtcgagcctagattattcgttgtttatttaggccagtgtgcagcccttaattgggcctttgagatccacttccaccgcccgatgggtaaatatcaaatgggtgaattattgttcacctttctaactaggatgagcaaaggggtattcaagattctcaccaacgcattaagtcacccccgggtttatcgaagattctgggacagtgggatgattgagccagtgtatggaccatcattggatacacaaaatctgcatcaaactgtttgcaacctcatctataactgctatgtcacttacttagaccttcttctcaatgatgatatcgataattaccctgttgttttatgtgaaagtgatgaggaggtggtgtcagatcgatttgataacattcaggctagacacttatgtataatcacagatctttattgtgatacaagagattgcccacctatcagaggtctctcccccgcagagaaatgcaaaacactgcttaaatttctgaggacccaagcacaattagacccgtccggtttatcttggaatgatcaaccgattttagtagatcaatattcctgctctttaacctaccttagaagaggatgcataaagcaaatcaggctaagggtagatcctgggtttgtgtcagctgcactctcagaatccaaagatccccctcgcattatgaggtcaggcaaacaggatacccagatgagcctaggacttgaaccccccttagacgatttactatcattaatgggatctattaacacaaatgcacacaatattcccgtgataggggaaggtctccgaaattacgaaatccatgcatttagaaggttgggtgtaaactcaacagcatgttataaagcagtagaaattgcatcattgatccgctgccattttgaagaaggtgaccaagggctgtttctcggggaagggtctggagcaatgctggttgcatataaggaaattctaaaattaggcagatgctattataacagtggtgtctctcctgattcccgaactggtcaacgggaactatccccgtacccatcagaagtgagtatggttgagcataaattatgtcttgaaaaatcagttagggtcctgttcaatggtcgacctgaagtaacctgggtaggtagtgtagattgttataagtatatcttaagccagatacctaccggtagtatagcggttatacactccgatattgagagcctccctgacaaagacgctgttgagaaactagaagagctttctgcaatcttatcgacggccctgatattaggcaaggttggctcagttttgattatcaagataatgcccattagcggggactgggtacagggtttcataaaattcgcactcgtgcattatcttaaagggataataatttacccgaggtacagtaattatgtgtcaactgaggcctatttagtattccaaggaatgcgagctagcagactggttaatcccgagggaatcaaacagcagattatgaggataggagtaaggacaacacctggattggtatgtcatatcctctccagaaaacaagacacgtgtgtccaagcctacatgggagtaccgtttaccaacgaagggttcaatcctaatctgaggtccctaacacctattgagaaagtcttggtgaactgtggtttttctgtcaatggagacaaggtctgtcgcactctgctacatcatgatatagtgtcagggtgcgaaggtttaacaaatgcagtgataatcctatatcgagagctggcaaggtttaaggaaatccatacgacatcccatgggatctttcatgcatacccagtgctaattgagagtcaacagcgggagttaatactagatatcaccagaaagataaccggccatattatcctgtactctagtaatctcactgacattgctcaagtaatcaagaacctcaaatccggatatttaacgattgatctattccaaaaccaattcacgagaaacatctccaagtcggatcgtgcactgattcggggaacaatagctcgcaggaactgggtgtttcaccttgagacaaaagaaatcaaagagtggtttaaattagtggggtatagtgctctgattcgagagtgatctatctactttctggaccctcacccacatccttgtatttagtgaaccgcaaaattgactaaaaaaaacaaaaagaagaaaaaagttcccttagccaattttgttt

SEQ ID NO: 45 - protein, 523 aa, Artificial
  synthetic
MASLLKTLSLFKKARDQPPTASGSGGALRGIKHVVIVPIPGDSTIETRSKLLDRLIKIIGEPDTSGPKQTGALIGLLSLFVESPGQLIQRIVDDPDISIKLVEVVPSDESKSGLTFASRGANLDAEADQYLGEDEYSSEEGAVQGWFENNEIRSVEVGDAEYFNIILASVLAQVWILLAKAVTAPDTAADSEMRRWVKYTQQRRVIGDFRMSRLWLDSVRNRIADDLSLRRFMVALILDIKRSPGTKPRIAEMICDIDNYIVEAGLASFILTIKFGIETMYPALGLHEFAGELTTIESLMILYQKMGETAPYMVILENSIQNKFSAGSYPLLWSYAMGVGVELENSMGGLNFGRTYFDPAYFRLGQEMVRRSAGKVSTSFAAELGISKEDAQLVSEIATRATDERVGRSTGVKQTQISFLHAEKPGKGIQSEDAIIAEGRPKKTRKKTSDRLSSDDIDDDVVGPTTSFDDGRRTDQDPDLDESSYDEKSAEAIAKMRLMTKLLGKTSPDTPTFRTYNDRELLN

SEQ ID NO: 46 - protein, 507 aa, Artificial
  synthetic
MAEEQAQHVSKGLECIKAIRDNPLCIEEIQEIKIIRDTVFNERESTVDSDQGEGDLDEDLDESQTATSGPARKGSVQSDPHGTRSMDGPHFKGSDGEGVGISTRSDIQHYYVHSHGGEEVTGIQDADSLMVPTDNNRGPDDDEGTDSADEGIDESSEDDPSKPTAHDRGFAPTPAATRSADVSTVMDDELTLLLGAHTKKKSREDPGRQLSFPKQPRIEPLEPRPESIKKGTGVKLISSGMAIDSTSINGATPSALGSPAKSSELSVPAANVPKSVPTAKMTLRSGPESGMHTQSEPAPVNEEEEEYEDELLAEILELKDAVTKINEDNQLILSKLDAVLSLKGEIDSIRKQLNKQNIAISTIEGHISSIMIAIPGFGKPDPDGLAESDVNPELRPIIGRDSGRALAEVLKKPPVTTGKTAQPSIKSGSKRQLLKEMRLQPINPRTSSAIKFNPGDDVPSKSVISSLIKSSSLDQSHKANMLTLLKEIKGDKNVKEFHQMVLEIIRA

SEQ ID NO: 47 - protein, 335 aa, Artificial
  synthetic
MTEVYDFDQSCWDSKGSLAPIFPNTYPDGRLIPQVRVIDPGLGDRKEECHMYLFLLGIIEDSQNEGPSTGRAFGSLPLGVGRSMAKPEELLKEAITLDIVVRRTAGVNEKLVFYNNTPLNVLLPWKKVLTNGSIFNANQVCGGVNLIPLDVSQRFRVVYMSLTRLSDDGCYRVPRGMLDFRSGNAIAFNILVTIQVNTVQTSGGDYISAPRGTIATFMVHIGNFKRKTNKQYSADYCKMKIEKMGLVFALGGIGGTSLHVRCTGKMSKALNAQLGLKKILCYPLMEVNEDLNRQLWRAECSITRVQAVLQPSVPNDFRIYNDVIINDNQGLFKIL

SEQ ID NO: 48 - protein, 557 aa, Artificial
  synthetic
MVKPKTALCPPTGIILEALLGFILVELSYSQVHWGNLSKIGVLSTESMTYKIMAPGASQYLVIKLMPNVTGLDNCTSSEVAEYESLLVNILGPINKALQVMTTSMRPAQTTYGSRRQKRFAGVVLAGAALGVATAAQITAGVALHQSNMNAQAIRDLKNSLEKSNQAIEEIRQATQEVVVAVQGVQDYINHELIPTISQMSCEIIGQKLGLKLLRYYTELLSIFGPSLRDPLSAEITIQALTYALGGDIYKVIEKLGYSGHDLAAVLESGGIKTRVVHADVQSKFIILSISYPTLSEVKGVVVHKLDVVSYNIGSQEWYTAVPKFVATNGYLISNFDESGCALVTGVSICSQNALYPMSSVMQQCMRGETGSCARTLVSGTIGNRFILSKGNIVANCASIMCKCTTSGTIINQDPDKLLTFIATDLCPLVEIEGITIQVGTREYPDTVYEHRINLGPVIPLEKLDVGTNLGNAISKLEDAKDLLDASNQILDLIQDSRMSIGKYIIWAAVGFSIFGGLLLLCCCKRYYQKVSANSTVINPTVKPDLTGTSRSYVKSL

SEQ ID NO: 49 - protein, 605 aa, Artificial
  synthetic
MPPLGDKSNAFYKDSARANLPILNINGFLPAHHRGPLLLLLATVSLVGLVALIAIAGVRFHKSTTMAKETNSLIQENIHHTDLLHHQVMDVLTPLFKIIGEEVGLRLPQRLAEIKQFILTKTSFFNPDREFDFRDLHWCINPPDRVKVNFSKFCQAIHIDNGIRLLGGIFIDQFLSHSKSDIFPGRVCPNGVTTRGEIISPALSLTSLSAKHLGLVDNIMFAMADGVYAKTYVLSHFSEGGVTETGEIRVFEIGLIRGWLGDLPVLHLTNFIHANVDSIGRHCTLAVGELKLASICTHARAIRVVRNVDSHPTSVSAISMGVFGSDYYQQSVEVIPVYDESVQQIHLSNHRGFIKDSKAYWAFPAEADTNSTRWSNCLTKVCTSRNLPFCNLTSWEPFNTTYPAVYAILVLEVSVHSDISMFIDESLGPLILNGYGMDLYSNSHEDQHWLTIPPKSGNVLGVINKLVVNGSAAVLPYILSYAASAGHEPCYAPVQHSLPSDTNTLAESNIVVLSTGKFRYVSATYDTSRSSHAIVYYIYNPTEGTSYFFPFRLTTRGIPAFLRIECFVWVEKIWCIHVYRYHSTVSKVEPVVETMVRLEFKCTSS

SEQ ID NO: 50 - protein, 2183 aa, Artificial
  synthetic
MDSISVNQILYPEVHLDSPIVTNKLVAILEYANLNHNYQLIDPTLIENIKQRINEGYSNQMIINYINVGHIINDLLATLPKTTHILYPNCNKDLFRAEDALVTQKLLDLFKKGNTIYTKISGGIIRCLTNLNQSLGLGGNLPEIYHNPIQDLGVMMQSSKWFGSFLFWFTIKTEMRSVIKSSTHNCQKRRYIPSFTTGKNFKILVSRDLVCLVNLADKRVYYLTFEMVLMYCDVIEGRLMTDTAMCIDSKFAQLRKRVHYLWDLIDGFFPDLGNSTYQLVALLEPLSLAYLQLKDATDQLKGAFLSHCFTEIKEILDNQGFTLPDTYAQLIEALDTIFITDDIHLTGEIFSFFRSFGHPRLEAVTAAENVRKHMNQPKVISYETLMKGHAIFCGIIISGFRDRHGGSWPPLILPEHASPVVKNAQASGEGLTYSQCVDNWKSFVGMRFKCFMPLSLDSDLTMYLKDKALSALRKEWDSVYPKEYLRYNPPKSSGSRRLVNVFLEDSEFDPYNMIMYVISGEYLRDPDFNLSYSLKEKEIKEVGRLFAKMTYKMRACQVIAENLIANGIGKYFKDNGMAKDEHDLTKALHTLAVSGVPKDKSQKHRDPRTKIDNHYNRETGGLPKCQLGGKQGPSGHRTGFISGRADDNDIDLYETVSAFITTDLKKYCLNWRYETISIYAQRLNEIYGLPSFFEWLHKRLEKSVLYVSDPHCPPDLDHHINLDEVPNQQIFIKYPMGGIEGYCQKLWTISTIPYLYLSAHECGVRIASLVQGDNQTIAVTKRVPSSWPYHLKKAEAARVTINYFMILRERLHDIGHHLKANETIISSHFFVYSKGIYYDGMLISQSLKSVARCVFWSETIVDETRAACSNISTTLAKSIEKGFDRYLAYALNLIKTIQQLLISLGFTINGSMTDDVVEPLLKDHRIITKMALLPAPIGGFNYLNMSRLFVRNIGDPVTSSLADLKRMIKANLLSETILHQVMTQAPGDSSFLDWASDPYSANLPCTQSMTRVLKNVTARHVLVNSPNPMLKGLFHSESQEEDETLASFLMDRGVIIPRAAHEILDNTITGAREAIAGMLDTTKGLIRASMQRGGLTPRIITRLSTYDYDQFKAGMRLFMGKGVNLLIDQDSCSVQLARALRNHMWATLAKGRPIYGLEVPDILESMKGQLIMRHESCNLCIQGSQNYGWFFVPSNCQLDNITSGTSSLRVPYIGSTTEERTDMKLSFVKSPSRSLKSAVRIATVYSWAYGDDEKSWNEAWQLAKQRANITLEELRMITPVSTSTNLAHRLRDKSTQVKYSGTSLIRVARYTTISNDNLSFTIAEKKVDTNFIYQQGMLLGLGILEHLFRLVDNTGPANTVLHLHVETECCVIPMTDYPSVPSLRQLRQSQTVCTNPLIYDKTPIIEKDAIRIYNQHHRRQVVEFVTWSTNQLYHVLAKSTAMSMIELVTKFEKDHLNEITTLIGDDDINSFITEFLLVEPRLFVVYLGQCAALNWAFEIHFHRPMGKYQMGELLFTFLTRMSKGVFKILTNALSHPRVYRRFWDSGMIEPVYGPSLDTQNLHQTVCNLIYNCYVTYLDLLLNDDIDNYPVVLCESDEEVVSDRFDNIQARHLCIITDLYCDTRDCPPIRGLSPAEKCKTLLKFLRTQAQLDPSGLSWNDQPILVDQYSCSLTYLRRGCIKQIRLRVDPGFVSAALSESKDPPRIMRSGKQDTQMSLGLEPPLDDLLSLMGSINTNAHNIPVIGEGLRNYEIHAFRRLGVNSTACYKAVEIASLIRCHFEEGDQGLFLGEGSGAMLVAYKEILKLGRCYYNSGVSPDSRTGQRELSPYPSEVSMVEHKLCLEKSVRVLFNGRPEVTWVGSVDCYKYILSQIPTGSIAVIHSDIESLPDKDAVEKLEELSAILSTALILGKVGSVLIIKIMPISGDWVQGFIKFALVHYLKGIIIYPRYSNYVSTEAYLVFQGMRASRLVNPEGIKQQIMRIGVRTTPGLVCHILSRKQDTCVQAYMGVPFTNEGFNPNLRSLTPIEKVLVNCGFSVNGDKVCRTLLHHDIVSGCEGLTNAVIILYRELARFKEIHTTSHGIFHAYPVLIESQQRELILDITRKITGHIILYSSNLTDIAQVIKNLKSGYLTIDLFQNQFTRNISKSDRALIRGTIARRNWVFHLETKEIKEWFKLVGYSALIRE